(19) United States
(12) Patent Application Publication
(10) Pub. No.: US 2013/0072495 A1
(43) Pub. Date: Mar. 21, 2013

(54) FUSED BICYCLIC KINASE INHIBITORS

(71) Applicants: An-Hu Li, Commack, NY (US); Mark J. Mulvihill, Dix Hills, NY (US); Arno G. Steinig, East Northport, NY (US)

(72) Inventors: An-Hu Li, Commack, NY (US); Mark J. Mulvihill, Dix Hills, NY (US); Arno G. Steinig, East Northport, NY (US)

(73) Assignee: OSI Pharmaceuticals, LLC

(21) Appl. No.: 13/697,819

(22) PCT Filed: May 16, 2011
(86) PCT No.: PCT/US2011/036573

§ 371 Date: Nov. 14, 2012

Related U.S. Application Data
(60) Provisional application No. 61/334,690, filed on May 14, 2010.

(51) Int. Cl. C07D 471/04; A61K 31/4985; A61P 35/00; A61K 31/4545; A61K 31/5025; C07D 487/04; A61K 31/437

(52) U.S. Cl. 514/249; 544/350; 546/113; 514/300; 546/119; 514/303; 544/236; 514/248

(57) ABSTRACT

Compounds of Formula I, as shown below and defined herein: pharmaceutically acceptable salts thereof, synthesis, intermediates, formulations, and methods of disease treatment therewith, including treatment of cancers, such as but not limited to tumors driven at least in part by at least one of RON, MET, IR, IGF-1R, or ALK. This Abstract is not limiting of the invention.

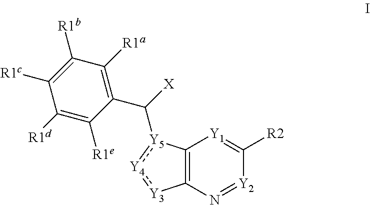

This application claims the benefit of U.S. Appl. No. 61/334,690 (filed May 14, 2010), which is incorporated herein in its entirety by this reference.

### FIELD AND BACKGROUND

The present invention pertains at least in part to cancer treatment, certain chemical compounds, and methods of treating tumors and cancers with the compounds.

RON (recepteur d'origine nantais) is a receptor tyrosine kinase that is part of the MET proto-oncogene family. It is activated by binding to its natural ligand MSP and signals via the PI3K and MAPK pathways. RON can be deregulated in cancer by mechanisms such as over-expression of the receptor and/or the presence of constitutively active splice variants. Inhibition of RON has been shown to lead to a decrease in proliferation, induction of apoptosis and affects cell metastasis. RON overexpression is observed in a variety of human cancers and exhibits increased expression with progression of the disease.

MET (also known as c-Met, cMet) is a receptor tyrosine kinase that is a heterodimeric protein comprising of a 50 kDa α-subunit and a 145 kDa β-subunit. Maggiora et al., J. Cell Physiol., 173:183-186 (1997). It is activated by binding to its natural ligand HGF (hepatocyte growth factor, also known as scatter factor) and signals via the PI3K and MAPK pathways. MET can be deregulated in cancer by mechanisms such as autocrine/paracrine HGF activation, over-expression of the receptor, and/or the presence of activating mutations. Significant expression of MET has been observed in a variety of human tumors, such as colon, lung, prostate (including bone metastases), gastric, renal, HCC, ovarian, breast, ESCC, and melanoma. Maulik et al., Cytokine & Growth Factor Rev., 13:41-59 (2002). MET is also implicated in atherosclerosis and lung fibrosis. Inhibition of MET can cause a decrease in cell motility, proliferation and metastasis, as reviewed in, e.g., Chem. & Eng. News, 85(34), 15-23 (2007).

Elevated expression of MET has been detected in numerous cancers including lung, breast, colorectal, prostate, pancreatic, head and neck, gastric, hepatocellular, ovarian, renal, glioma, melanoma, and some sarcomas. See Christensen et al., Cancer Letters, 225(1):1-26 (2005); Comoglio et al., Nature Rev. Drug Disc., 7(6):504-516 (2008). MET gene amplification and resulting overexpression has been reported in gastric and colorectal cancer. See Smolen et al., Proc. Natl. Acad. Sci. USA, 103(7):2316-2321 (2006); Zeng et al., Cancer Letters, 265(2):258-269 (2008). Taken together, the MET proto-oncogene has a role in human cancer and its over-expression correlates with poor prognosis. Abrogation of MET function with small molecule inhibitors, anti-MET antibodies or anti-HGF antibodies in preclinical xenograft model systems has shown impact when MET signaling serves as the main driver for proliferation and cell survival. See Comoglio et al., Nature Reviews Drug Disc., 7(6):504-516 (2008); Comoglio et al., Cancer & Metastasis Reviews, 27(1):85-94 (2008).

As human cancers progress to a more invasive, metastatic state, multiple signaling programs regulating cell survival and migration programs are observed depending on cell and tissue contexts. Gupta et al., Cell, 127:679-695 (2006). Recent data highlight the transdifferentiation of epithelial cancer cells to a more mesenchymal-like state, a process resembling epithelial-mesenchymal transition (EMT); (Oft et al., Genes & Dev., 10:2462-2477 (1996); Perl et al., Nature, 392:190-193 (1998)), to facilitate cell invasion and metastasis (Brabletz et al., Nature Rev., 5:744-749 (2005); Christofori, Nature, 41:444-450 (2006). Through EMT-like transitions mesenchymal-like tumor cells are thought to gain migratory capacity at the expense of proliferative potential. A mesenchymal-epithelial transition (MET) has been postulated to regenerate a more proliferative state and allow macrometastases resembling the primary tumor to form at distant sites Thiery, Nature Rev. Cancer, 2(6):442-454 (2002). MET and RON kinases have been shown to play a role in the EMT process. Camp et al., Cancer, 109(6):1030-1039 (2007); Grotegut et al., EMBO J., 25(15):3534-3545 (2006); Wang et al., Oncogene, 23(9):1668-1680 (2004). It has been documented in vitro that RON and MET can form heterodimers and signal via such RON-MET dimers.

MET and RON are known to interact and influence the activation of one another. Furthermore, co-expression of the two receptors, when compared to each receptor alone, is associated with the poorest clinical prognosis in bladder, CRC, and breast cancer patients. Since co-expression of RON and MET in cancer has been observed, such “cross-talk” may contribute to tumor growth.

ALK (Anaplastic Lymphoma Kinase) is a receptor tyrosine kinase that belongs to the insulin receptor subfamily. Constitutively active fusion proteins, activating mutations, or gene amplifications have been identified in various cancers, for example, kinase domain mutations in Neuroblastoma (Eng C., Nature, 455, 883-884 (2008)), echinoderm microtubule-associated protein-like 4 (EML4) gene—ALK fusion in non-small cell lung cancer (NSCLC) (Soda M. et al., Nature, 448, 561-566 (2007)), TPM3 and TPM4-ALK fusions in inflammatory myofibroblastic tumors (IMT) (Lawrence B. et al., Am. J. Pathol., 157, 377-384 (2000)), and nucleophosmin (NPM)—ALK fusions in anaplastic large cell lymphomas (ALCL) (Morris S. W. et al., Science, 263, 1281-1284 (1994)). Cell lines harboring such mutations or fusion proteins have been shown to be sensitive to ALK inhibition. McDermott U. et al., Cancer Res., 68, 3389-3395 (2008).

The following documents are also noted: WO10/104,945; WO10/059,771; WO10/039,248; WO09/140,549; WO09/094,123; WO08/124,849; WO08/53157; WO08/051,808; WO08/051,805; WO08/039,457; WO08/008,539; WO07/138,472; WO07/132,308; WO07/075,567; WO07/067,537; WO07/064,797; WO07/002,433; WO07/002,325; WO05/062795; WO05/010005; WO05/004607; WO03/82868; U.S. Pat. No. 7,585,876; U.S. Pat. No. 7,452,993; U.S. Pat. No. 7,259,154; U.S. Pat. No. 7,230,098; U.S. Pat. No. 6,235,769; US2010/256365; US2010/063031; US2009/143352; US2009/076046; US2009/005378; US2009/005356; US2008/293769; US2008/221197; US2008/221148; US2008/167338; US2007/032519; US2007/287711; US2007/123535; US2007/072874; US2007/066641; US2007/060633; US2007/049615; US2007/043068; US2007/032519; US2006/178374; US2006/128724; US2006/046991; US2005/182060; US2004/116488; U.S. Appl. No. 61/334,734 (filed May 14, 2010); Wang et al., J. Appl. Poly. Sci., 109(5), 3369-3375 (2008); Zou et al., Cancer Res., 67(9), 4408 (2007); Arteaga, Nature Medicine, 13, 6, 675 (June 2007); Engelman, Science, 316, 1039 (May 2007) Saucier, PNAS, 101, 2345 (February 2004).

There is a need for effective therapies for use in proliferative disease, including treatments for primary cancers, prevention of metastatic disease, and targeted therapies, including tyrosine kinase inhibitors, such as MET and/or RON and/or ALK inhibitors, dual inhibitors, including selective inhibitors (such as selectivity over Aurora kinase B and/or KDR), and for potent, orally bioavailable, and efficacious inhibitors, and inhibitors that maintain sensitivity of epithelial cells to epithelial cell directed therapies.

### SUMMARY

In some aspects, the present invention concerns compounds and salts thereof of Formula I, as shown below and defined herein:

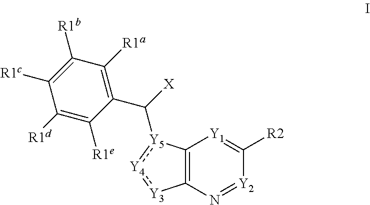

or a pharmaceutically acceptable salt thereof, wherein X is an optional substituent, Y₁-Y₅ are independently carbon or heteroatom, R1^{a}-R1^{e} are independently optional substituents, and R2 is an optional substituent.

The invention includes the compounds and salts thereof, and their physical forms, preparation of the compounds, useful intermediates, and pharmaceutical compositions and formulations thereof.

In some aspects, compounds of the invention are useful as inhibitors of kinases, including at least one of the MET, ALK, IR, IGF-1R, and RON kinases.

In some aspects, compounds of the invention are useful as inhibitors of kinases, including one or more of MET, ALK, IR, IGF-1R, RON, AXL, Tie-2, Flt3, FGFR3, Abl, Jak2, c-Src, Trk, PAK1, PAK2, and TAK1 kinases. In some aspects, compounds of the invention are inhibitors of kinases, including one or more of Blk, c-Raf, PRK2, Lck, Mek1, PDK-1, GSK3β, EGFR, p70S6K, BMX, SGK, and CaMKII kinases.

In some aspects, compounds of the invention are useful as selective inhibitors of one or more of MET, RON, ALK, IR, and IGF-1R. In some embodiments, the compound is useful as a selective inhibitor of MET and/or RON and/or ALK over other kinase targets, such as KDR and/or Aurora kinase B (AKB). In some aspects, compounds of the invention are useful as selective inhibitors of MET, RON, ALK with selectivity over KDR and Aurora kinase B (AKB).

In some aspects, compounds of the invention are useful in treating proliferative disease, particularly cancers, including cancers mediated by MET and/or RON and/or ALK, alone or in combination with other agents.

### DETAILED DESCRIPTION

### Compounds

In some aspects, the present invention concerns compounds and salts thereof of Formula I, above, wherein (Subgenus 1):

X is selected from H, C₁₋₃aliphatic or —OC₁₋₃aliphatic, either of which is optionally substituted with one or more halogen;

Y₁ and Y₂ are independently N or CH, except not more than one of Y₁ and Y₂ is N; Y₃ is NH or CH; and when Y₃ is NH, then at least one of Y₁, Y₂, and Y₄ is N and Y₅ is C; Y₄ is N or CH; Y₅ is N or C, except not more than one of Y₄ and Y₅ is N;

R^{1a}, R^{1b}, R^{1c}, R^{1d}, R^{1e} are each independently optional substituents selected from aliphatic, cyclic, O-aliphatic, O-cyclic, sulfide, sulfone, sulfoxide, amino, amido, carboxyl, acyl, ureido, S-cyclic, any of which is optionally substituted, halogen, or nitrile;

R2 is H or an optional substituent.

In some aspects of Formula I or Subgenus 1 thereof (Subgenus 2):

R^{1a}, R^{1b}, R^{1c}, R^{1d}, R^{1e} are each independently selected from H, halo, —CN, C₁₋₆ alkyl, —CF₃, —OCF₃, —OCHF₂, —OC₀₋₆alkyl, —S(O)ₘC₁₋₆alkyl, —SO₂N(C₀₋₆alkyl)(C₀₋₆alkyl), —N(C₀₋₆alkyl)(C₀₋₆alkyl), —N(C₀₋₆alkyl)C(═O)C₀₋₆alkyl, —N(C₀₋₆alkyl)C(═O)OC₀₋₆alkyl, —N(C₀₋₆alkyl)C(═O)N(C₀₋₆alkyl)(C₀₋₆alkyl), —C(═O)C₀₋₆alkyl, —C(═O)OC₀₋₆alkyl, —C(═O)N(C₀₋₆alkyl)(C₀₋₆alkyl), —O-heterocyclyl, —N(C₀₋₆alkyl)-heterocyclyl, —N(C₀₋₆alkyl)-heteroaryl, heterocyclyl, heteroaryl, —S-heteroaryl, or —O-heteroaryl; wherein the heterocyclyl is optionally substituted with oxo, C₁₋₆alkyl, C(═O)OC₁₋₆alkyl, C(═O)C₀₋₆alkyl, C(═O)N(C₀₋₆alkyl)(C₀₋₆alkyl), SO₂N(C₀₋₆alkyl)(C₀₋₆alkyl), or SO₂C₁₋₆alkyl; wherein the alkyl is optionally substituted with —OH, —OC₁₋₆alkyl, N(C₀₋₆alkyl)(C₀₋₆alkyl), C(═O)N(C₀₋₆alkyl)(C₀₋₆alkyl), C(═O)OC₀₋₆alkyl, C(═O)C₀₋₆alkyl, heterocyclyl, or heteroaryl;

R² is selected from H, halo, —CN, —CF₃, —NO₂, C₀₋₆alkyl, C₂₋₆alkenyl, C₂₋₆alkynyl, C₃₋₆cycloalkylC₀₋₆alkyl, C₃₋₆heterocycloalkylC₀₋₆alkyl, arylC₀₋₆alkyl, or heteroarylC₀₋₆alkyl, any of which is optionally substituted with one or more independent G¹ substituents;

or R² is selected from:

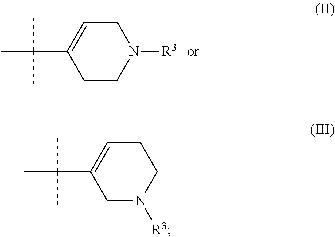

R³ is selected from H, C₁₋₁₂alkyl, R⁴O—C₂₋₁₂alkyl-, R⁴R⁵N—C₂₋₁₂alkyl-, R⁴S(O)ₘ—C₂₋₁₂alkyl, C₃₋₁₂cycloalkylC₀₋₁₂alkyl, C₃₋₁₂cycloalkenylC₁₋₁₂alkyl, heterocycloalkylC₀₋₁₂alkyl, arylC₀₋₁₂alkyl, heteroarylC₀₋₁₂alkyl, C₁₋₁₂alkylC₃₋₁₂cycloalkyl, C₃₋₁₂cycloalkylC₃₋₁₂cycloalkyl, C₃₋₁₂cycloalkenylC₃₋₁₂cycloalkyl, heterocycloalkylC₃₋₁₂cycloalkyl, arylC₃₋₁₂cycloalkyl, heteroarylC₃₋₁₂cycloalkyl, C₁₋₁₂alkyl-heterocycloalkyl, C₃₋₁₂cycloalkyl-heterocycloalkyl, C₃₋₁₂cycloalkenyl-heterocycloalkyl, heterocycloalkyl-heterocycloalkyl, aryl-heterocycloalkyl, heteroaryl-heterocycloalkyl, —C(O)R^{a}, R⁴O—C₀₋₁₂alkylC(O)—, R⁴R⁵N—C₀₋₁₂alkylC(O)—, R⁴S(O)ₘC₀₋₁₂alkylC(O)—, —CO₂R⁴, —C(O)NR⁴R⁵, —S(O)ₘR⁴, —SO₂NR⁴R⁵ or —C(S)OR⁴, any of which is optionally substituted with one or more independent G² substituents;

G¹ and G² are each independently selected from halo, —CN, —CF₃, —OCF₃, —NO₂, oxo, R⁶, C₁₋₁₂alkyl, C₂₋₁₂alkenyl, C₂₋₁₂alkynyl, C₃₋₁₂cycloalkylC₀₋₁₂alkyl, heterocycloalkylC₀₋₁₂alkyl, arylC₀₋₁₂alkyl, heteroarylC₀₋₁₂alkyl, —OR⁶, —S(O)ₘR⁶, —NR⁶R⁷, —SO₂NR⁶R⁷, —C(O)R^{b}, —C(O)NR⁶R⁷, —C(O)—C(O)NR⁶R⁷, —C(O)OR⁶, —C(O)—C(O)OR⁶, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, —NR¹⁰S(O)₂NR⁶R⁷, or —NR¹⁰S(O)NR⁶R⁷, any of which is optionally substituted with one or more independent Q¹ substituents;

For avoidance of doubt, a G1 cyclic group can include any multicyclic moieties, including bridged and spirocyclic systems where applicable. For example, a cycloaliphatic may include bicyclics such as bicyclo[3.1.0]hexyl, or spirocyclics such as spiro[3.3]heptyl. A heterocyclic may include bicyclics such as azabicyclo[3.2.1]octyl, or spirocyclics such as 2-azaspiro[3.3]heptyl, or 2,7-diazaspiro[3.5]nonyl. In case of bicyclics, such can be selected from carbobicyclic and heterobicyclic, any of which can be fused, bridged, or spirocyclic, and any of which is optionally substituted;

Q¹ is selected from halo, —CN, —NO₂, oxo, —CF₃, —OCF₃, arylC₀₋₁₂alkyl, heteroarylC₀₋₁₂alkyl, C₃₋₁₂cycloalkylC₀₋₁₂alkyl, heterocycloalkylC₀₋₁₂alkyl, arylC₃₋₁₂cycloalkyl, heteroarylC₃₋₁₂cycloalkyl, heterocycloalkylC₃₋₁₂cycloalkyl, C₃₋₁₂cycloalkylC₃₋₁₂cycloalkyl, C₁₋₁₂alkyl-heterocycloalkyl, heterocycloalkyl-heterocycloalkyl, aryl-heterocycloalkyl, heteroaryl-heterocycloalkyl, —C(O)—C(O)NR¹¹R¹², C(O)—C(O)OR¹¹, —OC(O)R^{c}, —NR¹¹C(O)R^{c}, —NR¹¹S(O)₂R¹², —(CR¹³R¹⁴)ₙC(O)R^{c}, (CR¹³R¹⁴)ₙC(O)OR¹¹, —(CR¹³R¹⁴)ₙC(O)NR¹¹R¹², —(CR¹³R¹⁴)ₙS(O)₂NR¹¹R¹², —(CR¹³R¹⁴)ₙN¹¹R¹², —(CR¹³R¹⁴)ₙOR¹¹, —(CR¹³R¹⁴)ₙS(O)ₘR¹¹, —NR¹⁵C(O)NR¹¹R¹², —NR¹⁵S(O)₂NR¹¹R¹² or —NR¹⁵S(O)NR¹¹R¹², any of which is optionally substituted with one or more independent Q² substituents;

Q² is selected from halo, —CN, —OH, —NH₂, —NO₂, oxo, —CF₃, —OCF₃, —CO₂H, —S(O)ₘH, arylC₀₋₁₂alkyl, heteroarylC₀₋₁₂alkyl, C₃₋₁₂cycloalkylC₀₋₁₂alkyl, heterocycloalkylC₀₋₁₂alkyl, arylC₃₋₁₂cycloalkyl, heteroarylC₃₋₁₂cycloalkyl, heterocycloalkylC₃₋₁₂cycloalkyl, C₃₋₁₂cycloalkylC₃₋₁₂cycloalkyl, C₁₋₁₂alkylheterocycloalkyl, heterocycloalkyl-heterocycloalkyl, aryl-heterocycloalkyl or heteroaryl-heterocycloalkyl, any of which is optionally substituted with one or more independent halo, —CN, —OH, —NH₂, or C₁₋₁₀alkyl which may be partially or fully halogenated, or —O—C₁₋₁₀alkyl which alkyl may be partially or fully halogenated;

each R⁴, R⁵, R⁶, R⁷, R⁸, R⁹, R¹⁰, R¹¹, R¹², R¹³, R¹⁴, R¹⁵, R^{a}, R^{b}, and R^{c} is independently selected from H, C₁₋₁₂alkyl or C₃₋₁₂cycloalkyl, each optionally substituted by halo, —OCF₃, or by —OC₀₋₃alkyl, arylC₀₋₁₂alkyl, heteroarylC₀₋₁₂alkyl, C₃₋₁₂cycloalkylC₀₋₁₂alkyl, heterocycloalkylC₀₋₁₂alkyl, arylC₃₋₁₂cycloalkyl, heteroarylC₃₋₁₂cycloalkyl, heterocycloalkylC₃₋₁₂cycloalkyl, C₃₋₁₂cycloalkylC₃₋₁₂cycloalkyl, C₁₋₁₂alkyl-heterocycloalkyl, heterocycloalkyl-heterocycloalkyl, aryl-heterocycloalkyl, or heteroaryl-heterocycloalkyl;

—NR⁴R⁵, —NR⁶R⁷ and —NR¹¹R¹² is each independently linear structure; or R⁴ and R⁵, or R⁶ and R⁷, or R¹¹ and R¹², respectively, can be taken together with the nitrogen atom to which they are attached to form a 3-12 membered saturated or unsaturated ring, wherein said ring optionally includes one or more heteroatoms selected from O, N, or S(O)ₘ;

—CR⁸R⁹ or —CR¹³R¹⁴ is each independently linear structure; or R⁸ and R⁹, or R¹³ and R¹⁴, respectively, can be taken together with the carbon atom to which they are attached to form a 3-12 membered saturated or unsaturated ring, wherein said ring optionally includes one or more heteroatoms selected from O, N, or S(O)ₘ;

n=0-7; and

m=0-2.

In some alternative embodiments, Y₁ and Y₂ are independently N or CH, except not more than one of Y₁ and Y₂ is N; Y₄ is N or CH, and Y₅ is N or C, except not more than one of Y₄ and Y₅ is N; Y₃ is NH or CH; wherein when Y₃ is NH, then at least one of Y₂, Y₄, and Y₅ is N. Alternatively, wherein Y₃ is NH, then at least one of Y₂ and Y₄ is N and Y₅ is C.

In some aspects of Formula I or Subgenus 1 or 2 thereof (Subgenus 3):

Y₁, Y₂, Y₃, and Y₄ are CH; and Y₅ is N; or

Y₁ and Y₂ are CH; Y₃ is NH; Y₄ is N; and Y₅ is C.

In some aspects of Formula I or Subgenus 1 or 2 thereof (Subgenus 4):

Y₁ is N; Y₂ and Y₄ are CH; Y₃ is NH; and Y₅ is C.

In some aspects of Formula I or Subgenera 1-4 thereof (Subgenus 5), X is selected from —OH, C₁₋₃alkyl, or C₁₋₃alkoxy.

In some aspects of Formula I or Subgenera 1, 3, or 4 thereof (Subgenus 6):

R^{1a} and R^{1e} are each independently selected from halo, —CN, C₁₋₆alkyl, —CF₃, —OCF₃, —OCHF₂, or —OC₀₋₆alkyl;

R^{1b}, R^{1c}, and R^{1d} are each independently selected from H, halo, —CN, C₁₋₆alkyl, —CF₃, —OCF₃, —OCHF₂, or —OC₀₋₆alkyl; wherein the alkyl is optionally substituted with —OH, —OC₁₋₆alkyl, N(C₀₋₆alkyl)(C₀₋₆alkyl), C(═O)N(C₀₋₆alkyl)(C₀₋₆alkyl), C(═O)OC₀₋₆alkyl, C(═O)C₀₋₆alkyl, or heteroaryl;

R² is selected from halo, —CN, —CF₃, —NO₂, C₁₋₆alkyl, C₂₋₆alkenyl, C₂₋₆alkynyl, C₃₋₆cycloalkylC₀₋₆alkyl, C₃₋₆heterocycloalkylC₀₋₆alkyl, arylC₀₋₆alkyl, or heteroarylC₀₋₆alkyl, any of which is optionally substituted with 1-3 independent G¹ substituents;

or R² is selected from:

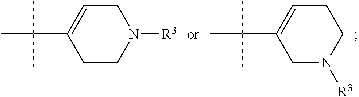

R³ is selected from H, C₁₋₁₂alkyl, R⁴O—C₂₋₁₂alkyl-, R⁴R⁵N—C₂₋₁₂alkyl-, R⁴S(O)ₘ—C₂₋₁₂alkyl-, C₃₋₁₂cycloalkylC₀₋₁₂alkyl, C₃₋₁₂cycloalkenylC₁₋₁₂alkyl, C₃₋₁₂heterocycloalkylC₀₋₁₂alkyl, arylC₀₋₁₂alkyl, heteroarylC₀₋₁₂alkyl, C₁₋₁₂alkylC₃₋₁₂cycloalkyl, C₃₋₁₂cycloalkylC₃₋₁₂cycloalkyl, C₃₋₁₂cycloalkenylC₃₋₁₂cycloalkyl, C₃₋₁₂heterocycloalkylC₃₋₁₂cycloalkyl, arylC₃₋₁₂cycloalkyl, heteroarylC₃₋₁₂cycloalkyl, C₁₋₁₂alkylC₃₋₁₂heterocycloalkyl, C₃₋₁₂cycloalkylC₃₋₁₂heterocycloalkyl, C₃₋₁₂cycloalkenylC₃₋₁₂heterocycloalkyl, C₃₋₁₂heterocycloalkylC₃₋₁₂heterocycloalkyl, arylC₃₋₁₂heterocycloalkyl, heteroarylC₃₋₁₂heterocycloalkyl, —C(O)R^{a}, R⁴O—C₀₋₁₂alkylC(O)—, R⁴R⁵N—C₀₋₁₂alkylC(O)—, R⁴S(O)ₘC₀₋₁₂alkylC(O)—, —CO₂R⁴, —C(O)NR⁴R⁵, —S(O)ₘR⁴, —SO₂NR⁴R⁵ or —C(S)OR⁴, any of which is optionally substituted with 1-2 independent G² substituents;

each G¹ is independently selected from halo, —CN, —CF₃, —OCF₃, —NO₂, R⁶, oxo, C₁₋₁₂alkyl, C₂₋₁₂alkenyl, C₂₋₁₂alkynyl, C₃₋₁₂cycloalkylC₀₋₁₂alkyl, C₃₋₁₂heterocycloalkylC₀₋₁₂alkyl, arylC₀₋₁₂alkyl, heteroarylC₀₋₁₂alkyl, —OR⁶, —S(O)ₘR⁶, —NR⁶R⁷, —SO₂NR⁶R⁷, —C(O)R^{b}, —C(O)NR⁶R⁷, —C(O)—C(O)NR⁶R⁷, —C(O)OR⁶, —C(O)—C(O)OR⁶, —OC(O)R^{b}, —NR⁶C(O)R^{b}, —NR⁶S(O)₂R⁷, —(CR⁸R⁹)ₙC(O)R^{b}, —(CR⁹R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, —NR¹⁰S(O)₂NR⁶R⁷, or —NR¹⁰S(O)NR⁶R⁷, any of which is optionally substituted with 1-2 independent Q¹ substituents;

each G² is independently selected from halo, —CN, —CF₃, —OCF₃, —NO₂, C₁₋₁₂alkyl, C₂₋₁₂alkenyl, C₂₋₁₂alkynyl, —OR⁶, —S(O)ₘR⁶, —NR⁶R⁷, —SO₂NR⁶R⁷, —C(O)R^{b}, —C(O)NR⁶R⁷, —C(O)—C(O)NR⁶R⁷, —C(O)OR⁶, —C(O)—C(O)OR⁶, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, —NR¹⁰S(O)₂NR⁶R⁷, or —NR¹⁰S(O)NR⁶R⁷, any of which is optionally substituted with 1-2 independent Q¹ substituents;

each Q¹ is selected from halo, —CN, —NO₂, oxo, —CF₃, —OCF₃, C₁₋₁₂alkyl, C₃₋₇cycloalkyl, —C(O)—C(O)NR¹¹R¹², —C(O)—C(O)OR¹¹, —OC(O)Rc, —NR¹¹C(O)Rc, —NR¹¹S(O)₂R¹², —(CR¹³R¹⁴)ₙC(O)R^{c}, —(CR¹³R¹⁴)ₙC(O)OR¹¹, —(CR¹³R¹⁴)ₙC(O)NR¹¹R¹², —(CR¹³R¹⁴)ₙS(O)₂NR¹¹R¹², —(CR¹³R¹⁴)ₙNR¹¹R¹², —(CR¹³R¹⁴)ₙOR¹¹, —(CR¹³R¹⁴)ₙS(O)ₘR¹¹, —NR¹⁵C(O)NR¹¹R¹², —NR¹⁵S(O)₂NR¹¹R¹² or —NR¹⁵S(O)NR¹¹R¹²;

each R⁴, R⁵, R⁶, R⁷, R⁸, R⁹, R¹⁰, R¹¹, R¹², R¹³, R¹⁴, R¹⁵, R^{a}, R^{b}, and R^{c} is independently C₀₋₁₂alkyl or C₃₋₇cycloalkyl, each independently optionally substituted by halo, —OCF₃, or —OC₀₋₃alkyl;

each —NR⁴R⁵, —NR⁶R⁷ and —NR¹¹R¹² is independently linear in structure; or R⁴ and R⁵, or R⁶ and R⁷, or R¹¹ and R¹², respectively, can be taken together with the nitrogen atom to which they are attached to form a 3-12 membered saturated or unsaturated ring, wherein said ring optionally includes one or more heteroatoms selected from O, N, or S(O)ₘ;

each —CR⁸R⁹ and —CR¹³R¹⁴ is independently linear in structure; or R⁸ and R⁹, or R¹³ and R¹⁴, respectively, can be taken together with the carbon atom to which they are attached to form a 3-12 membered saturated or unsaturated ring, wherein said ring optionally includes one or more heteroatoms selected from O, N, or S(O)ₘ;

n=0-4; and

m=0-2.

In some aspects of Formula I or Subgenera 1, 3, or 4 thereof (Subgenus 7), the compound has the formula:

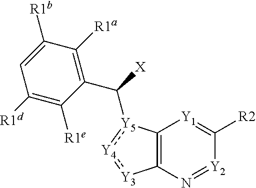

wherein X is methyl, ethyl, or methoxy;

R^{1a} and R^{1e} are each independently selected from halo, —CN, —CF₃, —OCF₃, —OCHF₂, or —OC₁₋₆alkyl;

R^{1b} and R^{1d} are each independently selected from H, halo, —CN, —CF₃, —OCF₃, —OCHF₂, or —OC₁₋₆alkyl;

(i) R² is phenyl or pyridinyl, each substituted by one or more R¹⁸ or G¹ wherein G¹ is ₄₋₇heterocycloalkyl optionally substituted with halogen, —OH, —OCH₃, or C₁₋₃alkyl, or G¹ is —C(O)NR⁶R⁷; wherein each R⁶ and R⁷ is independently C₀₋₃ alkyl; or NR⁶R⁷ defines a ₄₋₇heterocycloalkyl optionally substituted by C₁₋₆alkyl;

or (ii) R² is pyrazolo optionally substituted by one or more R¹⁸ or G¹ wherein G¹ is ₄₋₆heterocycloalkyl optionally substituted by halo, —R⁶, oxo, —S(O)ₘR⁶, —SO₂NR⁶R⁷, —C(O)R^{b}, —C(O)NR⁶R⁷, —C(O)—C(O)NR⁶R⁷, —C(O)OR⁶, or —C(O)—C(O)OR⁶; or G¹ is C₃₋₆cycloalkyl optionally substituted by halo, OH, —OR⁶, oxo, —S(O)ₘR⁶, —SO₂NR⁶R⁷, —C(O)R^{b}, —C(O)NR⁶R⁷, —C(O)—C(O)NR⁶R⁷, —C(O)OR⁶, or —C(O)—C(O)OR⁶; or —C₁₋₆alkyl which alkyl can be substituted by halo or —OC₀₋₅alkyl; or G¹ is C₁₋₆alkyl optionally substituted by —OH, —OR⁶, —R⁶, oxo, —NR⁶R⁷, —C(O)R^{b}, —C(O)NR⁶R⁷, —C(O)—C(O)NR⁶R⁷, —C(O)OR⁶, —C(O)—C(O)OR⁶, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)_{nOR}⁶, —(CR⁸R⁹)ₙS(O)ₘR⁶, —NR¹⁰C(O)NR⁶R⁷, —NR¹⁰S(O)₂NR⁶R⁷, or —NR¹⁹S(O)NR⁶R⁷; wherein each R⁶, R⁷, R⁸, R⁹, R¹⁰, and R^{b} is independently C₀₋₅alkyl or C₃₋₆cycloalkyl, each independently optionally substituted by halo, —OCF₃, or —OC₀₋₃alkyl; or NR⁶R⁷ defines a ₄₋₇heterocycloalkyl optionally substituted by C₁₋₆alkyl; R¹⁸ is —R⁶, halo, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, or —NR¹⁰S(O)₂NR⁶R⁷; and wherein each m is independently 0-2; each n is independently 0-2.

In some aspects of Formula I or Subgenus 7 thereof (Subgenus 8):

X is methyl;

R2 is pyrazole substituted by one or more R¹⁸ or G1;

R^{1a} and R^{1e} are each independently selected from halo, —CN, —CF₃, —OCF₃, —OCHF₂, or —OC₁₋₆alkyl;

R^{1b} and R^{1d} are each independently selected from H, halo, —CN, —CF₃, —OCF₃, —OCHF₂, or —OC₁₋₆alkyl;

G¹ is ₄₋₆heterocycloalkyl optionally substituted by halo, —R⁶, oxo, —S(O)ₘR⁶, —SO₂NR⁶R⁷, —C(O)R^{b}, —C(O)NR⁶R⁷, —C(O)—C(O)NR⁶R⁷, —C(O)OR⁶, or —C(O)—C(O)OR⁶;

or G¹ is ₃₋₆cycloalkyl optionally substituted by OH, —OR⁶, oxo, halo, —S(O)ₘR⁶, —SO₂NR⁶R⁷, —C(O)R^{b}, —C(O)NR⁶R⁷, —C(O)—C(O)NR⁶R⁷, —C(O)OR⁶, or —C(O)—C(O)OR⁶, or —C₁₋₆alkyl which alkyl can be substituted by halo or —OC₀₋₅alkyl;

or G¹ is C₁₋₆alkyl optionally substituted by —OH, —OR⁶, —R⁶, oxo, halo, —NR⁶R⁷, —C(O)R^{b}, —C(O)NR⁶R⁷, —C(O)—C(O)NR⁶R⁷, —C(O)OR⁶, —C(O)—C(O)OR⁶, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, —NR¹⁰S(O)₂NR⁶R⁷, or —NR¹⁰S(O)NR⁶R⁷;

wherein each R⁶, R⁷, R⁸, R⁹, R¹⁰, and R^{b} is independently C₀₋₅ alkyl or C₃₋₆cycloalkyl, each independently optionally substituted by halo, —OCF₃, or —OC₀₋₃alkyl; or NR⁶R⁷ defines a ₄₋₇heterocycloalkyl optionally substituted by C₁₋₆alkyl; R¹⁸ is —R⁶, halo, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)_{nOR}⁶, —(CR⁸R⁹)ₙS(O)ₘR⁶, —NR¹⁰C(O)NR⁶R⁷, or —NR¹⁰S(O)₂NR⁶R⁷; and

each m is independently 0-2; and each n is independently 0-2.

In some aspects of Formula I or Subgenus 7 thereof (Subgenus 9):

X is methyl;

R2 is pyrazole substituted by one or more R¹⁸ or G1;

R^{1a} is Cl;

R^{1e} is Cl, —OCH₃, or —OCHF₂;

each R^{1b} and R^{1d} is independently H, F, or —OCH₃;

G¹ is ₄₋₆heterocycloalkyl optionally substituted by halo, R⁶, oxo, —S(O)ₘR⁶, —SO₂NR⁶R⁷, —C(O)R^{b}, —C(O)NR⁶R⁷, —C(O)—C(O)NR⁶R⁷, —C(O)OR⁶, or —C(O)—C(O)OR⁶;

wherein each R⁶, R⁷, and R^{b} is independently C₀₋₅alkyl or C₃₋₆cycloalkyl, each independently optionally substituted by halo, —OCF₃, or —OC₀₋₃alkyl; or NR⁶R⁷ defines a ₄₋₇heterocycloalkyl optionally substituted by C₁₋₆alkyl; R¹⁸ is —R⁶, halo, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, (CR⁸R⁹)ₙS(O)₂NR⁶R⁷, —(CR⁸R⁹)ₙNR⁶R⁷, —(CR⁸R⁹)OR⁶, —(CR⁸R⁹)ₙS(O)ₘR⁶, —NR¹⁰C(O)NR⁶R⁷, or —NR¹⁰S(O)₂NR⁶R⁷; and

m is 0-2.

In some aspects of Formula I or Subgenus 7 thereof (Subgenus 10):

X is methyl;

R2 is pyrazole substituted by one or more R¹⁸ or G1;

R^{1a} is Cl;

R^{1e} is Cl, —OCH₃, or —OCHF₂;

each R^{1b} and R^{1d} is independently H, F, or —OCH₃;

G¹ is ₃₋₆cycloalkyl substituted by 0-2 substituents independently selected from —OH, —OR⁶, oxo, halo, —S(O)ₘR⁶, —SO₂NR⁶R⁷, —C(O)R^{b}, —C(O)NR⁶R⁷, —C(O)—C(O)NR⁶R⁷, —C(O)OR⁶, —C(O)—C(O)OR⁶, or —C₁₋₃alkyl which alkyl can be substituted by halo or —OC₀₋₅alkyl;

wherein each R⁶, R⁷, and R^{b} is independently C₀₋₅ alkyl or C₃₋₆cycloalkyl; or NR⁶R⁷ defines a ₄₋₇heterocycloalkyl optionally substituted by C₁₋₆alkyl; R¹⁸ is —R⁶, halo, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)_{nOR}⁶, —(CR⁸R⁹)ₙS(O)ₘR⁶, —NR¹⁰C(O)NR⁶R⁷, or —NR¹⁰S(O)₂NR⁶R⁷; and

m is 0-2.

In some aspects of Formula I or Subgenus 7 thereof (Subgenus 11):

X is methyl;

R² is pyrazole substituted by one or more R¹⁸ or G1;

R^{1a} is Cl;

R^{1e} is Cl, —OCH₃, or —OCHF₂;

each R^{1b} and R^{1d} is independently H, F, or —OCH₃;

G¹ is C₁₋₆alkyl substituted by 0-2 substituents independently selected from —OH, —OR⁶, —R⁶, oxo, halo, —NR⁶R⁷, —C(O)R^{b}, —C(O)NR⁶R⁷, —C(O)—C(O)NR⁶R⁷, —C(O)OR⁶, —C(O)—C(O)OR⁶, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, —NR¹⁰S(O)₂NR⁶R⁷, —NR¹⁰S(O)NR⁶R⁷, or ₄₋₇heterocycloalkyl optionally substituted by C₁₋₆alkyl;

wherein each R⁶, R⁷, R⁸, R⁹, R¹⁰, and R^{b} is independently C₀₋₅ alkyl or C₃₋₆cycloalkyl; or

NR⁶R⁷ defines a ₄₋₇heterocycloalkyl optionally substituted by C₁₋₆alkyl; R¹⁸ is —R⁶, halo, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, or —NR¹⁰S(O)₂NR⁶R⁷;

m is 0-2; and each n is independently 0-2.

In some aspects of Formula I or Subgenus 7 thereof (Subgenus 12):

X is methyl;

R2 is pyrazole substituted by one or more R¹⁸ or G1;

R^{1a} is Cl;

R^{1e} is Cl, —OCH₃, or —OCHF₂;

R^{1b} is F or —OCH₃;

R^{1d} is H;

G¹ is C₁₋₆alkyl substituted by 0-2 substituents independently selected from —OH, —OR⁶, —R⁶, oxo, halo, —NR⁶R⁷, —C(O)R^{b}, —C(O)NR⁶R⁷, —C(O)—C(O)NR⁶R⁷, —C(O)OR⁶, —C(O)—C(O)OR⁶, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, —NR¹⁰S(O)₂NR⁶R⁷, —NR¹⁰S(O)NR⁶R⁷, or ₄₋₇heterocycloalkyl optionally substituted by C₁₋₆alkyl;

wherein each R⁶, R⁷, R⁸, R⁹, R¹⁰, and R^{b} is independently C₀₋₅ alkyl or C₃₋₆cycloalkyl; or NR⁶R⁷ defines a ₄₋₇heterocycloalkyl optionally substituted by C₁₋₆alkyl; R¹⁸ is —R⁶, halo, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, or —NR¹⁰S(O)₂NR⁶R⁷;

m is 0-2; and each n is independently 0-2.

In some aspects of Formula I or Subgenus 7 thereof (Subgenus 13):

X is methyl;

R2 is pyrazole substituted by one or more R¹⁸ or G1;

R^{1a} is Cl;

R^{1e} is Cl, —OCH₃, or —OCHF₂;

R^{1b} is F;

R^{1d} is H;

G¹ is C₁₋₆alkyl substituted by 0-2 substituents independently selected from —OH, —OR⁶, —R⁶, oxo, halo; —NR⁶R⁷, —C(O)R^{b}, —C(O)NR⁶R⁷, —C(O)—C(O)NR⁶R⁷, —C(O)OR⁶, —C(O)—C(O)OR⁶, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, —NR¹⁰S(O)₂NR⁶R⁷, —NR¹⁰S(O)NR⁶R⁷, or ₄₋₇heterocycloalkyl optionally substituted by C₁₋₆alkyl;

wherein each R⁶, R⁷, R⁸, R⁹, R¹⁰, and R^{b} is independently C₀₋₃ alkyl or C₃₋₆cycloalkyl; or NR⁶R⁷ defines a ₄₋₇heterocycloalkyl optionally substituted by C₁₋₆alkyl; R¹⁸ is —R⁶, halo, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, or —NR¹⁰S(O)₂NR⁶R⁷;

m is 0-2; and each n is independently 0-2.

In some aspects of Formula I or Subgenus 7 thereof (Subgenus 14):

X is methyl;

R^{1a} and R^{1e} are each independently selected from halo, —CN, C₁₋₆alkyl, —CF₃, —OCF₃, —OCHF₂, or —OC₁₋₆alkyl;

R^{1b} and R^{1d} are each independently selected from H, halo, —CN, C₁₋₆alkyl, —CF₃, —OCF₃, —OCHF₂, or —OC₁₋₆alkyl;

R² is phenyl or pyridinyl, each substituted by G¹;

G¹ is ₄₋₇heterocycloalkyl optionally substituted with halogen, —OH, —OCH₃, or C₁₋₃alkyl;

or G¹ is —C(O)NR⁶R⁷; and

each R⁶ and R⁷ is independently C₀₋₃ alkyl or C₃₋₆cycloalkyl; or NR⁶R⁷ defines a ₄₋₇heterocycloalkyl optionally substituted by C₁₋₆alkyl.

In some aspects of Formula I or Subgenus 7 thereof (Subgenus 15):

X is methyl;

R^{1a} is Cl;

R^{1e} is Cl, —OCH₃, or —OCHF₂;

R^{1b} is F or —OCH₃;

R^{1d} is H;

R² is selected from

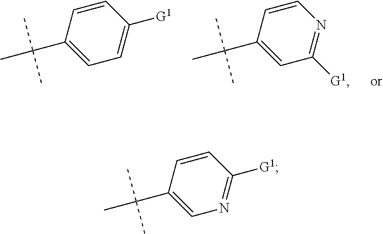

and G¹ is selected from piperazine, homopiperazine, morpholine, piperidine, azetidine, or pyrrolidine, each optionally substituted with halogen, —OH, —OCH₃, or C₁₋₃alkyl or C₃₋₆cycloalkyl.

In some aspects of Formula I or Subgenus 7 thereof (Subgenus 16):

X is methyl;

R^{1a} is Cl;

R^{1e} is Cl, —OCH₃, or —OCHF₂;

R^{1b} is F or —OCH₃;

R^{1d} is H;

R² is selected from

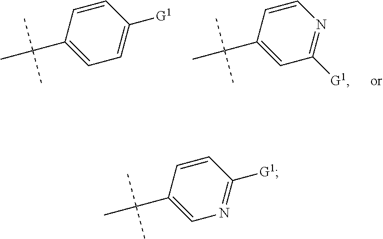

G¹ is NR⁶R⁷;

wherein each R⁶ and R⁷ is independently C₀₋₃ alkyl or C₃₋₆cycloalkyl; or NR⁶R⁷ defines a ring selected from piperazine, homopiperazine, morpholine, piperidine, azetidine, or pyrrolidine, each optionally substituted with halogen, —OH, —OCH₃, C₁₋₃alkyl, or C₃₋₆cycloalkyl.

In some aspects of Formula I or Subgenus 7 thereof (Subgenus 17):

X is methyl;

wherein R^{1a} and R^{1e} are each independently selected from halo, —CN, C₁₋₆alkyl, —CF₃, —OCF₃, —OCHF₂, or —OC₁₋₆alkyl;

R^{1b} and R^{1d} are each independently selected from H, halo, —CN, C₁₋₆alkyl, —CF₃, —OCF₃, —OCHF₂, or —OC₁₋₆alkyl;

R² is selected from

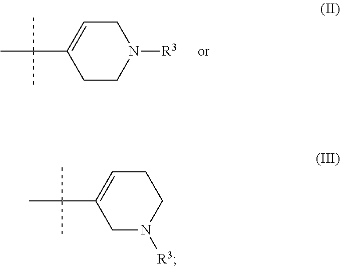

wherein R³ is selected from —R⁴, —C(O)R^{a}, R⁴O—C₀₋₁₂alkylC(O), R⁴R⁵N—C₀₋₁₂alkylC(O), —CO₂R⁴, —C(O)NR⁴R⁵, —S(O)ₘR⁴, —SO₂NR⁴R⁵, or —C(S)OR⁴);

each R^{a}, R⁴, and R⁵ is independently C₀₋₃alkyl or C₃₋₆cycloalkyl; or NR⁴R⁵ defines a ₄₋₇heterocycloalkyl optionally substituted by C₁₋₆alkyl;

each m is independently 0-2.

In some aspects of Formula I or Subgenera 1-17 thereof (Subgenus 18), the compound or salt is present as a material that is substantially free of its (S)-1-(phenyl)ethyl enantiomer when Y₄ or Y₅ of Formula I is N and substantially free of its (R)-1-(phenyl)ethyl enantiomer when Y₄ or Y₅ is not N.

In some aspects, the compound or salt thereof is selected from any one of the Examples herein.

Each variable definition above includes any subset thereof and the compounds of Formula I include any combination of such variables or variable subsets.

In some aspects, the invention includes a compound of Formula I or a pharmaceutically acceptable salt thereof, in any of the above recitations, which further exhibits inhibition of MET in a cellular mechanistic assay with an IC₅₀ of about 10 nM or less, 100 nM or less, 200 nM or less, or 400 nM or less.

In some aspects, the invention includes a compound of Formula I or a pharmaceutically acceptable salt thereof, in any of the above recitations, which further exhibits inhibition of RON in a cellular assay with an IC₅₀ of about 500 nM or less or 200 nM or less or 100 nM or less or 10 nM or less.

In some aspects, the invention includes a compound of Formula I or a pharmaceutically acceptable salt thereof, in any of the above recitations, which exhibits both inhibition of MET in a cellular assay with an IC₅₀ as above and inhibition of RON in a cellular assay with an IC₅₀ as above.

In some aspects, the invention includes a compound of Formula I or a pharmaceutically acceptable salt thereof, in any of the above recitations, which is about 10-fold or more selective for MET over KDR and/or over AKB.

The invention includes a compound of Formula I or a pharmaceutically acceptable salt thereof, which is sufficiently orally bioavailable for effective oral human administration.

The invention includes a compound of Formula I or a pharmaceutically acceptable salt thereof, which has a suitable therapeutic window for effective human administration, oral or otherwise.

In some aspects, the invention includes any of the compound examples herein and pharmaceutically acceptable salts thereof.

The invention includes the compounds and salts thereof, and their physical forms, preparation of the compounds, useful intermediates, and pharmaceutical compositions and formulations thereof.

The compounds of the invention and term “compound” in the claims include any pharmaceutically acceptable salts or solvates, and any amorphous or crystal forms, or tautomers, whether or not specifically recited in context.

The invention includes the isomers of the compounds. Compounds may have one or more asymmetric carbon atoms can exist as two or more stereoisomers. Where a compound of the invention contains an alkenyl or alkenylene group, geometric cis/trans (or Z/E) isomers are possible. Where the compound contains, for example, a keto or oxime group or an aromatic moiety, tautomeric isomerism (‘tautomerism’) can occur. A single compound may exhibit more than one type of isomerism.

The present invention includes any stereoisomers, even if not specifically shown, individually as well as mixtures, geometric isomers, and pharmaceutically acceptable salts thereof. Where a compound or stereocenter is described or shown without definitive stereochemistry, it is to be taken to embrace all possible individual isomers, configurations, and mixtures thereof. Thus, a material sample containing a mixture of stereoisomers would be embraced by a recitation of either of the stereoisomers or a recitation without definitive stereochemistry. Also contemplated are any cis/trans isomers or tautomers of the compounds described.

Included within the scope of the invention are all stereoisomers, geometric isomers and tautomeric forms of the inventive compounds, including compounds exhibiting more than one type of isomerism, and mixtures of one or more thereof.

When a tautomer of the compound of Formula (I) exists, the compound of formula (I) of the present invention includes any possible tautomers and pharmaceutically acceptable salts thereof, and mixtures thereof, except where specifically stated otherwise.

The compounds of the invention are not limited to those containing all of their atoms in their natural isotopic abundance. The present invention includes compounds wherein one or more hydrogen, carbon or other atoms are replaced by different isotopes thereof. Such compounds can be useful as research and diagnostic tools in metabolism pharmacokinetic studies and in binding assays. A recitation of a compound or an atom within a compound includes isotopologs, i.e., species wherein an atom or compound varies only with respect to isotopic enrichment and/or in the position of isotopic enrichment. For nonlimiting example, in some cases it may be desirable to enrich one or more hydrogen atoms with deuterium (D) or to enrich carbon with ¹³C. Other examples of isotopes suitable for inclusion in the compounds of the invention include isotopes of hydrogen, chlorine, fluorine, iodine, nitrogen, oxygen, phosphorus, and sulfur. Certain isotopically-labeled compounds of the invention may be useful in drug and/or substrate tissue distribution studies. Substitution with heavier isotopes such as deuterium may afford certain therapeutic advantages resulting from greater metabolic stability, for example, increased in vivo half-life or reduced dosage requirements, and hence may be preferred in some circumstances. Substitution with positron emitting isotopes may be useful in Positron Emission Topography (PET) studies for examining substrate receptor occupancy.

Further, the compounds may be amorphous or may exist or be prepared in various crystal forms or polymorphs, including solvates and hydrates. The invention includes any such forms provided herein, at any purity level. A recitation of a compound per se means the compound regardless of any unspecified stereochemistry, physical form and whether or not associated with solvent or water.

The compounds of the invention may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when the solvent is water. Pharmaceutically acceptable solvates in accordance with the invention include hydrates and solvates wherein the solvent of crystallization may be isotopically substituted, e.g., D₂O, d6-acetone, d 6-DMSO.

Also included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionized, partially ionized, or non-ionized.

The invention includes prodrugs of compounds of the invention which may, when administered to a patient, be converted into the inventive compounds, for example, by hydrolytic cleavage. Prodrugs in accordance with the invention can, for example, be produced by replacing appropriate functionalities present in the inventive compounds with certain moieties known to those skilled in the art as ‘pro-moieties’ as known in the art. Particularly favored derivatives and prodrugs of the invention are those that increase the bioavailability of the compounds when such compounds are administered to a patient, enhance delivery of the parent compound to a given biological compartment, increase solubility to allow administration by injection, alter metabolism or alter rate of excretion.

A pharmaceutically acceptable salt of the inventive compounds can be readily prepared by mixing together solutions of the compound and the desired acid or base, as appropriate. The salt may precipitate from solution and be collected by filtration or may be recovered by evaporation of the solvent. The degree of ionization in the salt may vary from completely ionized to almost non-ionized.

Compounds that are basic are capable of forming a wide variety of salts with various inorganic and organic acids. The acids that can be used to prepare pharmaceutically acceptable acid addition salts of such basic compounds are those that form acceptable acid addition salts. When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Other salts are aspartate, besylate, bicarbonate/carbonate, bisulphate/sulfate, borate, camsylate, edisylate, gluceptate, glucuronate, hexafluorophosphate, hibenzate, hydrobromide/bromide, hydroiodide/iodide, malonate, methylsulfate, naphthylate, 2-napsylate, nicotinate, orotate, oxalate, palmitate, phosphate/hydrogen, phosphate/dihydrogen, phosphate, saccharate, stearate, tartrate, tosylate, and trifluoroacetate.

When the compound of the present invention is acidic, its corresponding salt can be conveniently prepared from pharmaceutically acceptable bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Salts derived from pharmaceutically acceptable organic bases include salts of primary, secondary, and tertiary amines, as well as cyclic amines and substituted amines such as naturally occurring and synthesized substituted amines. Other pharmaceutically acceptable organic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. Other examples include benzathine, diolamine, glycine, meglumine, and olamine.

### Compound Preparation

The invention includes the intermediates, examples, and synthetic methods described herein.

The compounds of the Formula I may be prepared by the methods described below, together with synthetic methods known in the art of organic chemistry, or modifications and derivatizations that are familiar to those of ordinary skill in the art. The starting materials used herein are commercially available or may be prepared by routine methods known in the art [such as those methods disclosed in standard reference books such as the Compendium of Organic Synthetic Methods, Vol. I-VI (Wiley-Interscience); or the Comprehensive Organic Transformations, by R. C. Larock (Wiley-Interscience)]. Preferred methods include, but are not limited to, those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991, and T. W. Greene and P. G. M. Wuts, Protective Groups in Organic Chemistry, John Wiley & Sons, 1999, which are hereby incorporated by reference.

Compounds of Formula I, or their pharmaceutically acceptable salts, can be prepared according to the reaction Schemes discussed hereinbelow and the general skill in the art. Unless otherwise indicated, the substituents in the Schemes are defined as above. Isolation and purification of the products is accomplished by standard procedures, which are known to a chemist of ordinary skill.

When a general or exemplary synthetic procedure is referred to, one skilled in the art can readily determine the appropriate reagents, if not indicated, extrapolating from the general or exemplary procedures. Some of the general procedures are given as examples for preparing specific compounds. One skilled in the art can readily adapt such procedures to the synthesis of other compounds. Representation of an unsubstituted position in structures shown or referred to in the general procedures is for convenience and does not preclude substitution as described elsewhere herein. For specific groups that can be present, either as R groups in the general procedures or as optional substituents not shown, refer to the descriptions in the remainder of this document, including the claims, summary and detailed description.

### General Synthesis

Unless otherwise indicated, the substituents in the Schemes are defined as above. Isolation and purification of the products is accomplished by standard procedures, which are known to a chemist of ordinary skill. In the following general descriptions, R¹ indicates one or more substituents R^{1a}-R^{1e}.

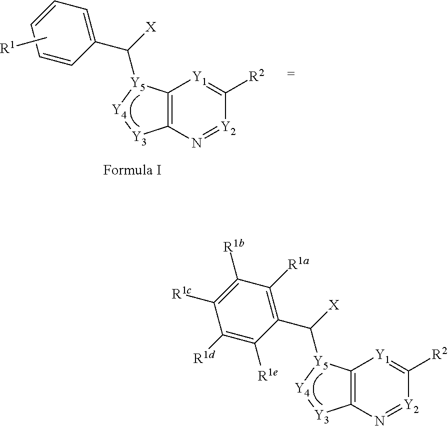

Compounds of Formula Ia (also known as 7-azaindoles or pyrrolo[2,3-b]pyridines) are compounds of Formula I wherein Y3=NH, Y5=C, and Y2, Y4 and Y1=CH. These compounds, or their pharmaceutically acceptable salts, can be prepared according to the reaction Schemes discussed hereinbelow and the general skill in the art.

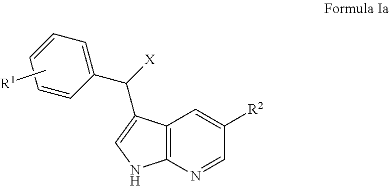

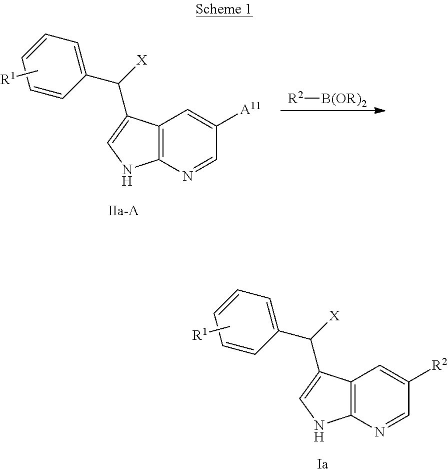

Compounds of Formula Ia can be prepared from IIa-A as in Scheme 1, wherein R¹ and R² are as defined previously, A¹¹ is halogen such as Cl, Br, or I, or trifluoromethanesulfonate, and B(OR)₂ is a suitable boronic acid/ester. In a typical preparation of compounds of Formula Ia, a compound of Formula IIa-A is reacted with a suitable boronic acid/ester (R²—B(OR)₂) in a suitable solvent via typical Suzuki coupling procedures. Suitable solvents for use in the above process include, but are not limited to, ethers such as THF, glyme, dioxane, dimethoxyethane, and the like; DMF; DMSO; MeCN; alcohols such as MeOH, EtOH, isopropanol, trifluoroethanol, and the like; and chlorinated solvents such as DCM or chloroform (CHCl₃). If desired, mixtures of these solvents can be used; however, preferred solvents are dimethoxyethane/water and dioxane/water. The above process can be carried out at temperatures between about 0° C. and about 120° C. Preferably, the reaction is carried out between 60° C. and about 100° C. The above process is preferably carried out at about atmospheric pressure although higher or lower pressures can be used. Substantially equimolar amounts of reactants are preferably used although higher or lower amounts can be used. One skilled in the art will appreciate that alternative methods may be applicable for preparing compounds of Formula Ia from IIa-A. For example, compound of Formula IIa-A could be reacted with a suitable organotin reagent R²—SnBu₃ or the like in a suitable solvent via typical Stille coupling procedures.

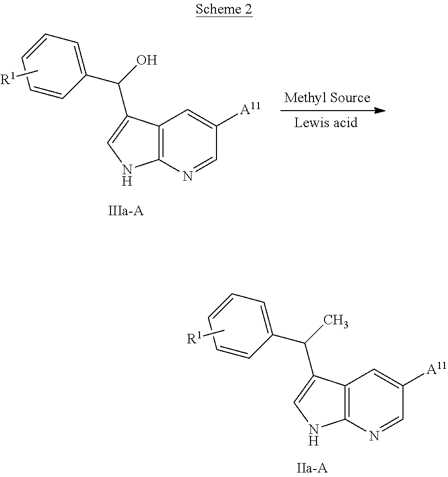

Compounds of Formula IIa-A can be prepared as in Scheme 2, wherein R¹ is as defined previously and A¹¹ is halogen such as Cl, Br, or I, or trifluoromethanesulfonate. In a typical preparation IIIa-A can be reacted with a suitable methyl source in the presence of a Lewis acid in a suitable solvent. Suitable methyl source for use in the above process include, but are not limited to Me₃Al, Me₂Zn, Me₂AlCl, methyl Grignard reagents. A preferred methyl source is Me₂Zn. The methyl source may also be generated in situ, such as by reacting a methyl Grignard reagent with zinc chloride and using the resulting reagent without isolation for the above process. Suitable Lewis acids for use in the above process include, but are not limited to BF₃.OEt₂, AlCl₃, TiCl₄, and the like. A preferred Lewis acid is BF₃.OEt₂. Suitable solvents for use in the above process include, are not limited to, ethers such as THF, glyme, and the like; DMF; DMSO; MeCN; toluene; cyclohexane, and chlorinated solvents such as DCM or chloroform (CHCl₃). If desired, mixtures of these solvents can be used; however, a preferred solvent is THF. The above process can be carried out at temperatures between about −78° C. and about 120° C. Preferably, the reaction can be carried out between 40° C. and about 70° C. An excess amount of the methyl source and Lewis acid are preferably used.

Compounds similar to those of Formula IIIa-A wherein the hydroxy group is replaced with an alkoxy group may also be used for the above process using the same Lewis acids and methyl source.

Compounds similar to those of Formula IIa-A wherein the methyl group is replaced by an alkyl group can be prepared by replacing the methyl source with an alkyl source under otherwise similar reaction conditions. For example, an ethyl group may be introduced using reagents such as Et₂Zn, and a propyl group may be introduced using reagents such as PrZnBr.

Compounds of Formula Ia wherein X=CN may be prepared by reacting compounds of Formula IIIa-A with a suitable cyanide source in the presence of a suitable Lewis acid, followed by reacting with a boronic acid/ester R²—B(OR)₂ via Suzuki coupling procedures as described above in Scheme 1. Suitable reagents for the cyanation include, but are not limited to, TMSCN as cyanide source, InBr₃ as Lewis acid, and chlorinated solvents such as DCM. Preferably, the cyanation may be carried out at temperatures between about 0° C. and about 60° C.

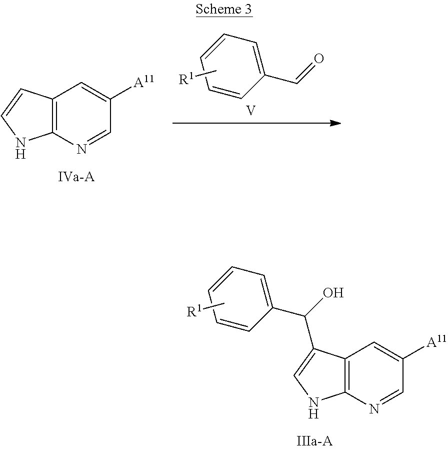

Compounds of Formula IIIa-A can be prepared as in Scheme 3, wherein R¹ is as defined previously and A¹¹ is halogen such as Cl, Br, or I. In a typical preparation, IVa-A is treated with benzaldehyde V in a suitable solvent in the presence of a suitable base at a suitable reaction temperature. Suitable solvents for use in the above process include, but are not limited to, ethers such as THF, glyme, and the like; DMF, DMSO; MeCN; chlorinated solvents such as DCM or chloroform (CHCl₃); and alcohols such as MeOH, EtOH, isopropanol, or trifluoroethanol. If desired, mixtures of these solvents can be used or no solvent can be used. A preferred solvent is MeOH. Suitable bases for use in the above process include, but are not limited to, KOH, NaOH, LiOH, KOtBu, NaOtBu and NaHMDS and the like. A preferred base is KOH. The above process can be carried out at temperatures between about −78° C. and about 120° C. Preferably, the reaction is carried out between 20° C. and about 60° C. The above process to produce compounds of the present invention is preferably carried out at about atmospheric pressure although higher or lower pressures can be used. Substantially equimolar amounts of reactants are preferably used although higher or lower amounts can be used.

When alcohols are used as solvent, analogs of compounds of Formula IIIa-A wherein the hydroxyl group is replaced with an alkoxy group can also be obtained. For example, with MeOH as solvent one can obtain the methoxy analogs.

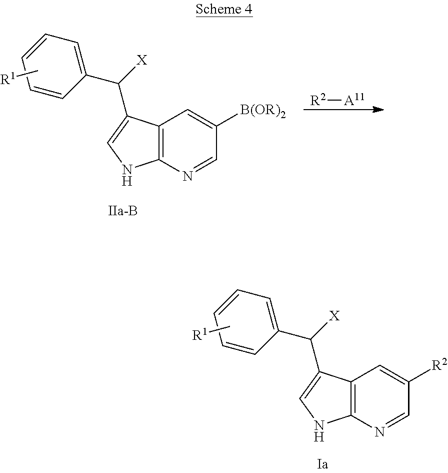

Compounds of Formula Ia can be prepared as in Scheme 4, wherein R¹ and R² are as defined previously, A¹¹ is halogen such as Cl, Br, or I, or trifluoromethanesulfonate, and B(OR)₂ is a suitable boronic acid/ester. Compound IIa-B can be reacted with a suitable coupling partner (R²-A¹¹) in a suitable solvent via typical Suzuki coupling procedures. Suitable solvents for use in the above process include, but are not limited to, ethers such as THF, glyme, dioxane, dimethoxyethane, and the like; DMF; DMSO; MeCN; alcohols such as MeOH, EtOH, isopropanol, trifluoroethanol, and the like; and chlorinated solvents such as DCM or chloroform (CHCl₃). If desired, mixtures of these solvents can be used, however, a preferred solvent is dimethoxyethane/water. The above process can be carried out at temperatures between about −78° C. and about 120° C. Preferably, the reaction is carried out between 60° C. and about 100° C. The above process is preferably carried out at about atmospheric pressure although higher or lower pressures can be used. Substantially, equimolar amounts of reactants are preferably used although higher or lower amounts can be used if desired.

One skilled in the art will appreciate that alternative methods may be applicable for preparing compounds of Formula Ia from R²-A¹¹, e.g., via typical Stille coupling procedures.

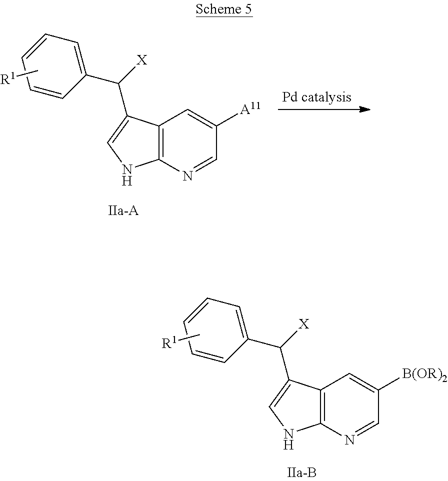

Compounds of Formula IIa-B can be prepared as in Scheme 5, wherein R¹ is as defined previously, A¹¹ is halogen such as Cl, Br, or I, or trifluoromethanesulfonate, and B(OR)₂ is a suitable boronic acid/ester. In a typical preparation a compound of Formula IIa-A can be reacted with a suitable coupling partner (Bis(pinacolato)diboron or Pinacolborane)) in a suitable solvent under Palladium catalysis. Suitable solvents for use in the above process include, but are not limited to, ethers such as THF, glyme, dioxane, dimethoxyethane, and the like; DMF; DMSO; MeCN; alcohols such as MeOH, EtOH, isopropanol, trifluoroethanol, and the like; and chlorinated solvents such as DCM or chloroform (CHCl₃). If desired, mixtures of these solvents can be used; however, preferred solvents are dioxane or DMSO. The above process can be carried out at temperatures between about 0° C. and about 120° C. Preferably, the reaction is carried out between 60° C. and about 100° C. The above process is preferably carried out at about atmospheric pressure although higher or lower pressures can be used. Substantially equimolar amounts of reactants used although higher or lower amounts can be used if desired.

One skilled in the art will appreciate that alternative methods may be applicable for preparing compounds of Formula IIa-B. For example, via halogen-metal exchange (for example, halogen-lithium exchange) and quench with borylation reagents such as tri-isopropyl borate.

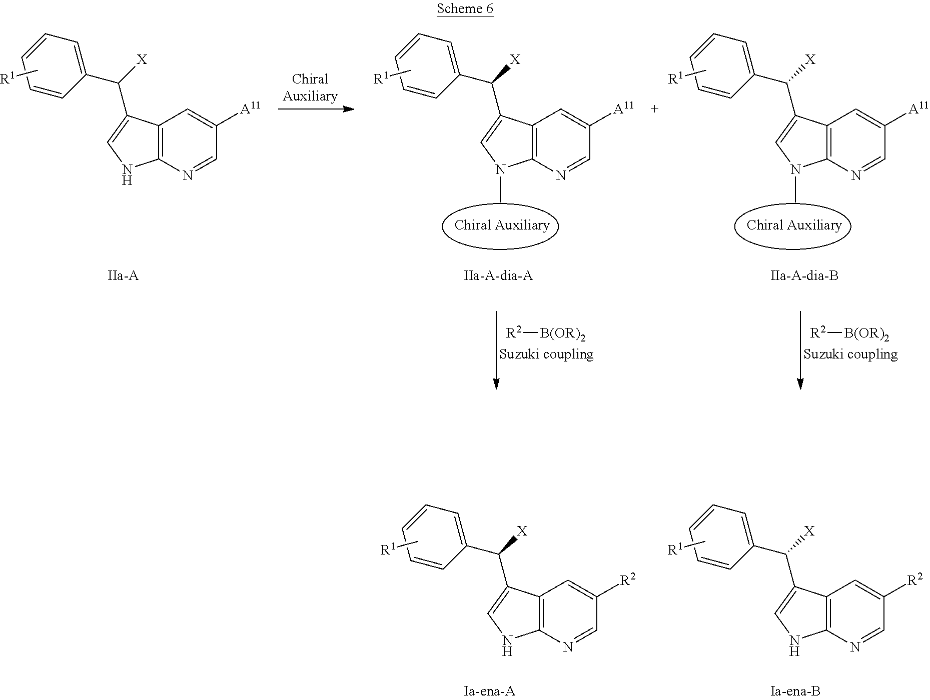

Chiral resolution: Compounds of Formula Ia have the carbon chiral center shown in Scheme 6. The enantiomerically pure isomers Ia-ena-A and Ia-ena-B can be prepared by a chiral resolution through a chemical reaction which leads to two diastereomers IIa-A-dia-A and IIa-A-dia-B. After separation of these two diastereomers by flash chromatography or crystallization, each diastereomer can be subjected to a Suzuki coupling as shown in Scheme 6 to produce Ia-ena-A and Ia-ena-B individually.

In a typical preparation of IIa-A-dia-A and IIa-A-dia-B, a compound of Formula IIa-A is reacted with a chiral auxiliary in the presence of a coupling reagent to provide both IIa-A-dia-A and IIa-A-dia-B, which are separated by chromatography. Suitable chiral auxiliaries for use in the above process include, but are not limited to amino acids and their derivatives, (1S)-(+)-camphor-10-sulfonic acid, (1R)-(−)-camphor-10-sulfonic acid and the like. However, a preferred chiral auxiliary is Fmoc-L-Leucine. Suitable solvents for use in the above process included, but are not limited to, ethers such as THF, glyme, dioxane, dimethoxyethane, and the like; DMF; DMSO; MeCN; alcohols such as MeOH, EtOH, isopropanol, trifluoroethanol, and the like; and chlorinated solvents such as DCM or chloroform (CHCl₃). If desired, mixtures of these solvents can be used; however, a preferred solvent is DMF. The suitable coupling reagents for use in the above process include, but are not limited to DCC, EDC, TBTU, HBTU and the like. A preferred coupling reagent is TBTU. The above process can be carried out at temperatures between about −78° C. and about 120° C. Preferably, the reaction is carried out between 0° C. and about 60° C. The above process is preferably carried out at about atmospheric pressure although higher or lower pressures can be used if desired. Substantially equimolar amounts of reactants are preferably used although higher or lower amounts can be used if desired.

After purification and separation, both IIa-A-dia-A and IIa-A-dia-B are reacted separately with a suitable boronic acid/ester (R²—B(OR)₂), to provide both Ia-ena-A and Ia-ena-B, via typical Suzuki coupling procedures as in Scheme 1.

One skilled in the art will appreciate that instead of covalently attaching a chiral auxiliary to compound IIa-A one may form diastereomeric salts that may be separated by crystallization. Neutralization of the separated diastereomeric salts provides the separated enantiomers of IIa-A. Suitable chiral auxiliaries include, but are not limited to amino acids and their derivatives, (1S)-(+)-camphor-10-sulfonic acid, (1R)-(−)-camphor-10-sulfonic acid and the like.

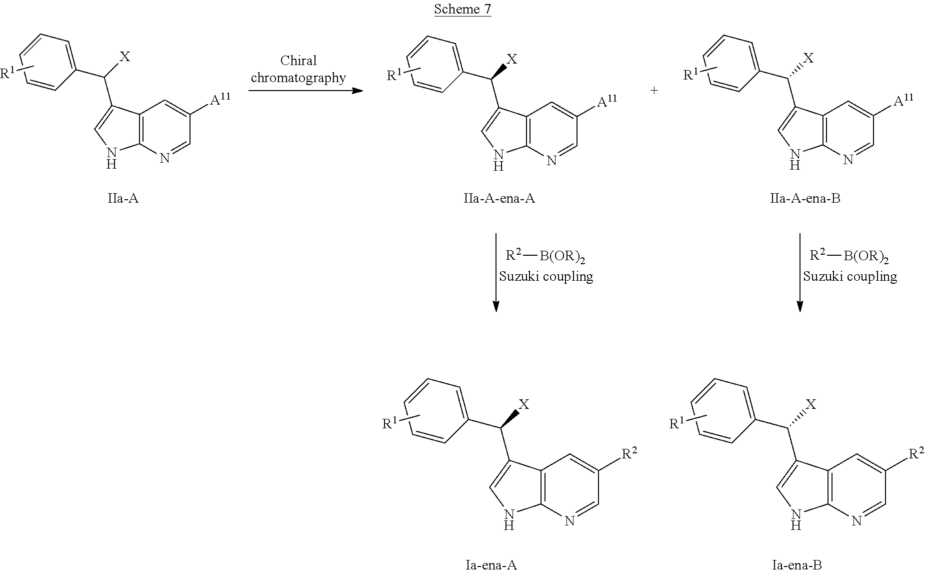

Alternatively, the enantiomerically pure isomers Ia-ena-A and Ia-ena-B can be prepared as in Scheme 7 individually from corresponding enantiomerically pure IIa-A-ena-A and IIa-A-ena-B through Suzuki coupling reactions. Enantiomerically pure IIa-A-ena-A and IIa-A-ena-B can be prepared from separation of racemic mixture IIa-A by a chiral chromatography as in Scheme 7.

The suitable system for separation of IIa-A-ena-A and IIa-A-ena-B by chromatography can be, but is not limited to, chiral HPLC (high performance liquid chromatography) systems, chiral SFC (supercritical fluid chromatography) systems and the like. After separation, both IIa-A-ena-A and IIa-A-ena-B can be reacted individually with a suitable boronic acid/ester (R²—B(OR)₂), to provide both Ia-ena-A and Ia-ena-B, via typical Suzuki coupling procedures as in Scheme 1.

As will be apparent to the skilled artisan, the synthetic route/sequence can be modified as desired for the preparation of a given compound. For example, Group R² can be installed on compound IVa-A under conditions similar to Schemes 1, 5, and 4. The resulting compound can be treated with an appropriate benzaldehyde under conditions similar to Scheme 3, followed by introduction of a methyl group similar to Scheme 2.

A skilled artisan will realize that the reactions shown in Schemes 1, 4-7 can be conducted under similar conditions with compounds in which the methyl group shown is replaced by other alkyl or alkoxy groups within the scope defined for the variable X.

Compounds of Formula Ib {also known as 4-azaindoles or pyrrolo[3,2-b]pyridines} are compounds of Formula I wherein Y5=N, and Y2, Y3, Y4 and Y1=CH. These compounds, or their pharmaceutically acceptable salts, can be prepared according to the reaction Schemes discussed hereinbelow and the general skill in the art.

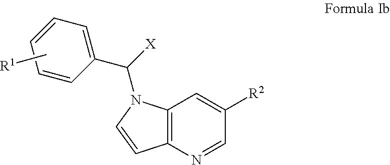

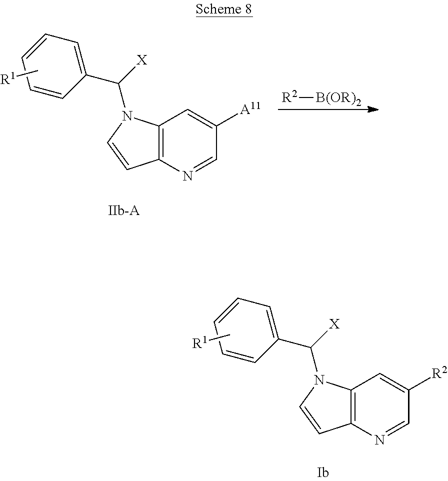

Compounds of Formula Ib can be prepared from IIb-A as in Scheme 8, wherein R¹ and R² are as defined previously, X is C₁₋₃alkyl, A¹¹ is halogen such as Cl, Br, or I, or trifluoromethanesulfonate, and B(OR)₂ is a suitable boronic acid/ester. In a typical preparation of compounds of Formula Ib, a compound of Formula IIb-A is reacted with a suitable boronic acid/ester (R²—B(OR)₂) in a suitable solvent via typical Suzuki coupling procedures, applying reaction conditions substantially similar to those described for compounds of Formula Ia. One skilled in the art will appreciate that alternative methods may be applicable for preparing compounds of Formula Ib from IIb-A. For example, compound of Formula IIb-A could be reacted with a suitable organotin reagent R²—SnBu₃ or the like in a suitable solvent via typical Stille coupling procedures.

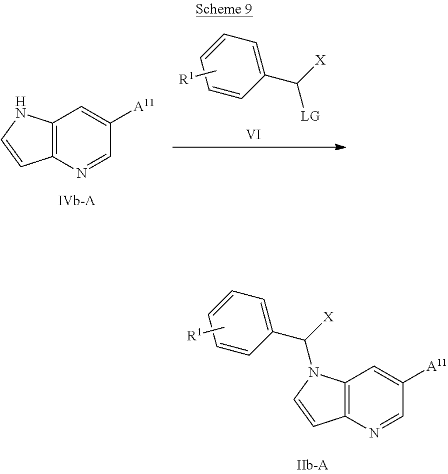

Compounds of Formula IIb-A can be prepared from IVb-A as in Scheme 9, wherein R¹ is as defined previously, X is C₁₋₃alkyl and A¹¹ is halogen such as Cl, Br, or I, or trifluoromethanesulfonate, and LG is a suitable leaving group such as halogens Cl, Br, or I, or suitable sulfonate esters such as mesylate, tosylate, or triflate. In a typical preparation, IVb-A is treated with VI in a suitable solvent in the presence of a suitable base at a suitable reaction temperature. Suitable solvents for use in the above process include, but are not limited to, ethers such as THF, glyme, and the like; DMF, DMSO; MeCN. If desired, mixtures of these solvents can be used or no solvent can be used. Preferred solvents are THF and DMF. Suitable bases for use in the above process include, but are not limited to, KOH, NaOH, LiOH, NaH, KOtBu, NaOtBu and NaHMDS and the like. A preferred base is NaH. The above process can be carried out at temperatures between about −78° C. and about 120° C. Preferably, the reaction is carried out between 20° C. and about 60° C. The above process to produce compounds of the present invention is preferably carried out at about atmospheric pressure although higher or lower pressures can be used. Substantially equimolar amounts of reactants are preferably used although higher or lower amounts can be used.

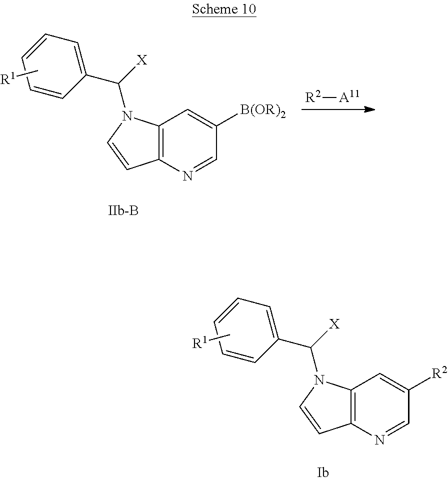

Compounds of Formula Ib can also be prepared as in Scheme 10, wherein R¹ and R² are as defined previously, A¹¹ is halogen such as Cl, Br, or I, or trifluoromethanesulfonate, and B(OR)₂ is a suitable boronic acid/ester. Compound IIb-B can be reacted with a suitable coupling partner (R²-A¹¹) in a suitable solvent via typical Suzuki coupling procedures. Suitable solvents for use in the above process include, but are not limited to, ethers such as THF, glyme, dioxane, dimethoxyethane, and the like; DMF; DMSO; MeCN; and alcohols such as MeOH, EtOH, isopropanol, trifluoroethanol, and the like. If desired, mixtures of these solvents can be used; however, a preferred solvent system is dimethoxyethane/water. The above process can be carried out at temperatures between about 0° C. and about 120° C. Preferably, the reaction is carried out between 60° C. and about 100° C. The above process is preferably carried out at about atmospheric pressure although higher or lower pressures can be used. Substantially, equimolar amounts of reactants are preferably used although higher or lower amounts can be used if desired.

One skilled in the art will appreciate that alternative methods may be applicable for preparing compounds of Formula Ib from R²-A¹¹, e.g., via typical Stille coupling procedures.

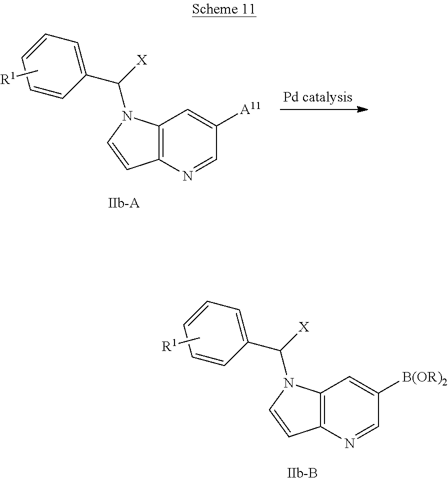

Compounds of Formula IIb-B can be prepared as in Scheme 11, wherein R¹ is as defined previously, A¹¹ is halogen such as Cl, Br, or I, or trifluoromethanesulfonate, and B(OR)₂ is a suitable boronic acid/ester. In a typical preparation a compound of Formula IIb-A can be reacted with a suitable coupling partner (Bis(pinacolato)diboron or Pinacolborane)) in a suitable solvent under Palladium catalysis. Suitable solvents for use in the above process include, but are not limited to, ethers such as THF, glyme, dioxane, dimethoxyethane, and the like; DMF; DMSO; MeCN; and alcohols such as MeOH, EtOH, isopropanol, trifluoroethanol. If desired, mixtures of these solvents can be used; however, preferred solvents are DMSO or dioxane. The above process can be carried out at temperatures between about 0° C. and about 120° C. Preferably, the reaction is carried out between 60° C. and about 100° C. The above process is preferably carried out at about atmospheric pressure although higher or lower pressures can be used. Substantially equimolar amounts of reactants used although higher or lower amounts can be used if desired.

One skilled in the art will appreciate that alternative methods may be applicable for preparing compounds of Formula IIb-B. For example, via halogen-metal exchange (for example, halogen-Lithium exchange) and quench with borylation reagents such as tri-isopropyl borate.

As will be apparent to the skilled artisan, the synthetic route/sequence can be modified as desired for the preparation of a given compound. For example, Group R² can be installed on compound IVb-A under conditions similar to Schemes 8, 10, and 11.

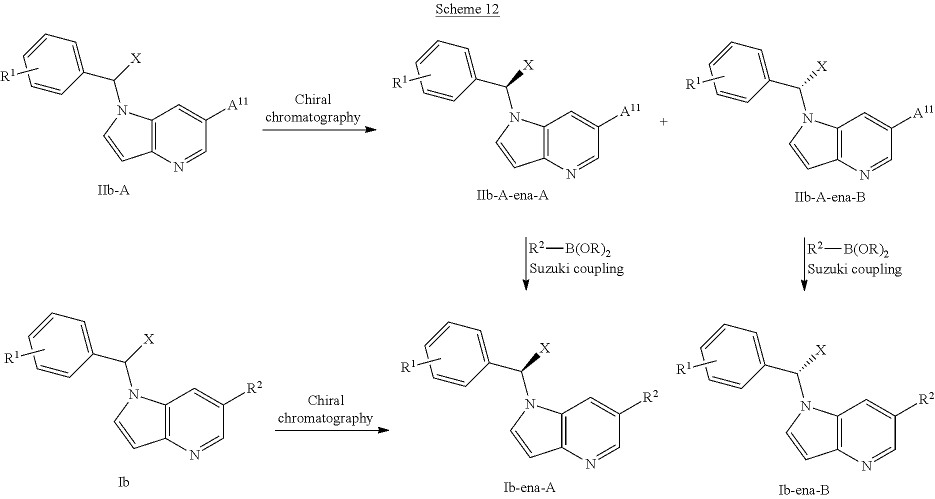

Compounds of Formula Ib have a chiral center at the carbon atom that connects the 4-azaindole core with X and the phenyl ring substituted with R1. Enantiomerically pure IIb-A-ena-A and IIb-A-ena-B can be prepared by separation of racemic mixture IIb-A by chromatography with an enantiomerically pure stationary phase as in Scheme 12. Similarly, enantiomerically pure Ib-A-ena-A and Ib-A-ena-B can be prepared by separation of racemic mixture Ib. Suitable chromatography systems for separation of racemic IIb or Ib include, but are not limited to, chiral HPLC (high performance liquid chromatography) systems, chiral SFC (supercritical fluid chromatography) systems and the like.

One skilled in the art will appreciate that instead of separating the enantiomers by chromatographic means one may form diastereomeric salts that may be separated by crystallization. Neutralization of the separated diastereomeric salts provides the separated enantiomers of IIb or Ib. Suitable chiral auxiliaries include, but are not limited to amino acids and their derivatives, (1S)-(+)-camphor-10-sulfonic acid, (1R)-(−)-camphor-10-sulfonic acid and the like.

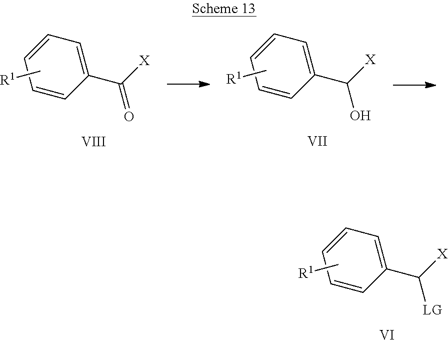

Alternatively, enantiomerically enriched/pure IIb-A-ena-A and IIb-A-ena-B may be obtained by using enantiomerically pure VI for the reaction shown in Scheme 9. Compounds of Formula VI may be obtained as shown in Scheme 13 from ketones VIII by reduction to give the alcohols VII, which are then converted to VI under typical conditions known to the skilled artisan. Racemic compounds VII and VI may be separated into their enantiomers by the chromatographic methods described above. Alternatively, enantiomerically enriched VII may be obtained directly from VIII by using enantiopure reducing agents. Enzymatic resolution of VII may also be used to obtain enantiomerically enriched VII by converting VII to its acetate ester and using a suitable enzyme to hydrolyze one enantiomer in preference over the other.

Compounds of Formula Ic {also known as pyrazolo[3,4-b]pyridines} are compounds of Formula I wherein Y4=N, Y3=NH, Y5=C and Y2, Y1=CH. These compounds, or their pharmaceutically acceptable salts, can be prepared according to the reaction schemes discussed hereinbelow and the general skill in the art.

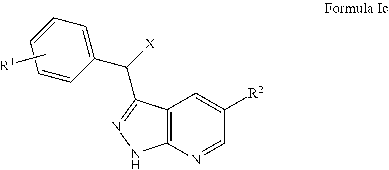

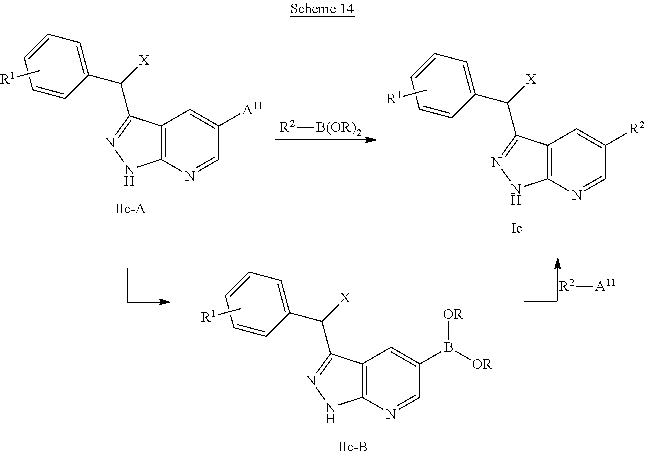

Compounds of Formula Ic can be prepared from IIc-A as in Scheme 14, wherein R¹ and R² are as defined previously, X is C₁₋₃alkyl, A¹¹ is halogen such as Cl, Br, or I, or trifluoromethanesulfonate, and B(OR)₂ is a suitable boronic acid/ester. In a typical preparation of compounds of Formula Ic, a compound of Formula IIc-A is reacted with a suitable boronic acid/ester [R²—B(OR)₂] in a suitable solvent via typical Suzuki coupling procedures, applying reaction conditions substantially similar to those described for compounds of Formula Ia. One skilled in the art will appreciate that alternative methods may be applicable for preparing compounds of Formula Ic from IIc-A. For example, compound of Formula IIc-A could be reacted with a suitable organotin reagent R²—SnBu₃ or the like in a suitable solvent via typical Stille coupling procedures. Alternatively, a compound of Formula IIc-A may first be converted to a boronic acid/ester of formula IIc-B, followed by reaction with R²-A¹¹ via typical Suzuki coupling procedures, applying conditions substantially similar to those described for compounds of Formula Ia in Schemes 4 and 5. One skilled in the art will appreciate that alternative methods may be applicable for preparing compounds of Formula Ic from R²-A¹¹, e.g., via typical Stille coupling procedures.

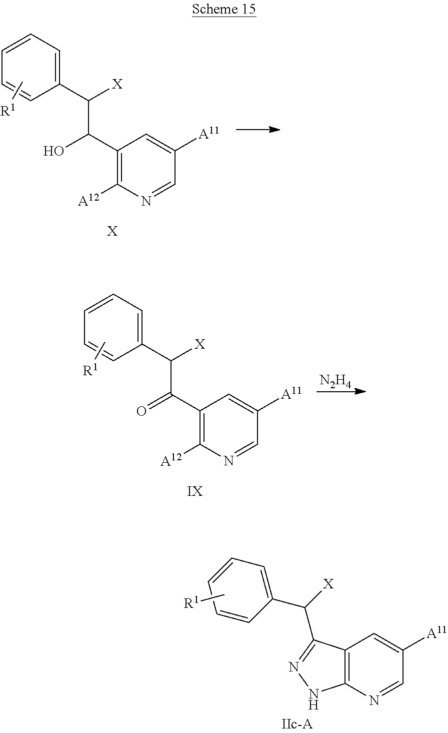

Compounds of Formula IIc-A can be prepared as in Scheme 15, wherein R¹ is as defined previously, X is C₁₋₃alkyl, A¹¹ is halogen such as Cl, Br, or I, and A¹² is F or Cl. The secondary alcohol in compounds of Formula IX can be oxidized by a variety of methods using, e.g., metal-based oxidants such as pyridinium chlorochromate or sulfur-based oxidants such as in the Swern reaction, under conditions known to the skilled artisan. Reaction of compounds of Formula IX with hydrazine gives compounds of Formula IIc-A. This reaction can be conducted with anhydrous hydrazine or hydrazine hydrate. Typical solvents for this reaction include alcoholic solvents, such as ethanol or isopropanol, although other solvents can be used. The reaction can be carried out at temperatures between about 0° C. and about 140° C. Preferably, the reaction is carried out near the reflux temperature of the solvent. Higher temperatures can be used when the reaction is conducted in a sealed vessel.

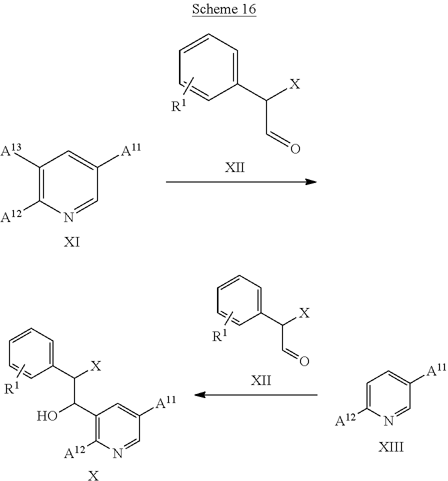

Compounds of Formula X can be prepared from XI or XIII as in Scheme 16 wherein R¹ is as defined previously, X is C₁₋₃alkyl, A¹¹ is halogen such as Cl, Br, or I, A¹² is F or Cl, and A¹³ is Br or I. Selective halogen-metal exchange of A¹³ in XI using organolithium or magnesium reagents generates an anion that is reacted with the aldehyde XII. A preferred reagent XI is 5-bromo-2-chloro-3-iodopyridine, and the halogen-metal exchange is conducted with iPrMgCl in THF at about 50° C. Another suitable reagent XI is 3-bromo-2,5-dichloropyridine, and the halogen-metal exchange is conducted with nBuLi at about 70° C. Alternatively, the anion may be generated by deprotonation of XIII at C3, which is then reacted with the same aldehyde XII to furnish the compound of Formula X. A preferred reagent XIII is 5-bromo-2-fluoropyridine, and the deprotonation may be conducted with LDA in THF at about −75° C.

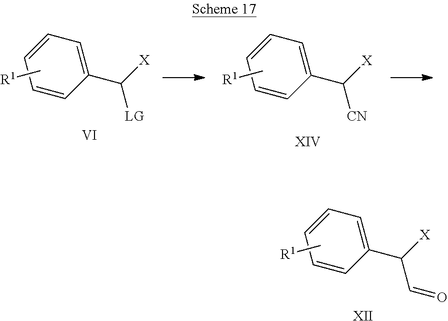

Compounds of Formula XII may be prepared as shown in Scheme 17, wherein R¹ is as defined previously, X is C₁₋₃alkyl, and LG is a suitable leaving group such as halogens Cl, Br, or I, or suitable sulfonate esters such as mesylate, tosylate, or triflate. The leaving group LG in compounds of Formula VI may be displaced with cyanide to obtain compound XIV. Suitable reaction conditions include, but are not limited to, heating VI with NaCN in DMF at about 60-90° C. The nitrile group is then reduced to furnish the aldehyde XII. Suitable reaction conditions include, but are not limited to, reacting XIV with diisobutylaluminum hydride in toluene at about 0-60° C. Depending on the R¹ substituents, the skilled artisan will decide whether or not other reaction conditions may be more suitable.

Compounds of Formula Ic have a chiral center at the carbon atom that connects the pyrazolopyridine core with X and the phenyl ring substituted with R1. Enantiomerically pure compounds Ic and IIc can be prepared by separation of the racemic mixtures by chromatography on an enantiomerically pure stationary phase as described for compounds of Formula Ib and IIb in Scheme 12. Alternatively, compounds of Formula Ic or IIc may be reacted with a chiral auxiliary to provide diastereomers that are separated by chromatography, followed by removal of the chiral auxiliary, as described in Scheme 6 for compounds of Formula IIa. Furthermore, one may form diastereomeric salts that may be separated by crystallization. Neutralization of the separated diastereomeric salts provides the separated enantiomers of IIc or Ic.

Compounds of Formula Id {also known as pyrrolo[2,3-b]pyrazines} are compounds of Formula I wherein Y3=NH, Y5=C, Y1=N and Y2, Y4=CH. These compounds, or their pharmaceutically acceptable salts, can be prepared according to the reaction Schemes 1-7 discussed for the compounds of Formula Ia and the general skill in the art.

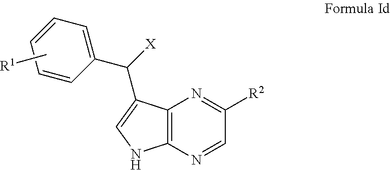

Compounds of Formula Id have a chiral center at the carbon atom that connects the pyrrolopyrazine core with X and the phenyl ring substituted with R1. Enantiomerically pure compounds Id can be prepared by the methods discussed for the compounds of Formula Ia and the general skill in the art.

Compounds of Formula Ie {also known as pyrrolo[2,3-c]pyridazines} are compounds of Formula I wherein Y3=NH, Y5=C, Y2=N, and Y4 & Y1=CH. These compounds, or their pharmaceutically acceptable salts, can be prepared according to the reaction Schemes discussed hereinbelow and the general skill in the art.

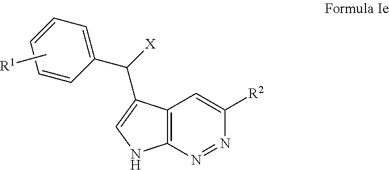

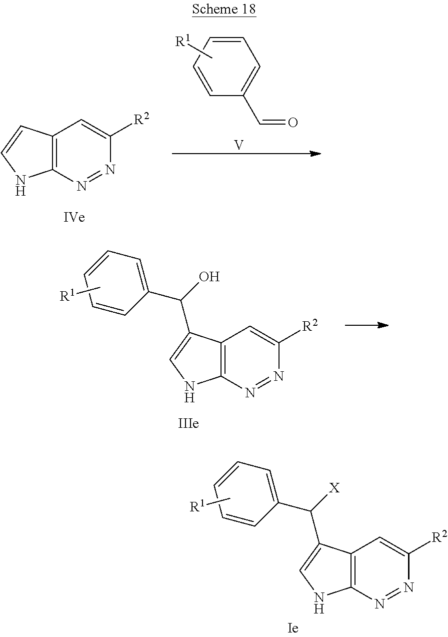

Compounds of Formula Ie wherein X=C₁₋₃alkyl can be prepared from IVe as in Scheme 18, wherein R¹ and R² are as defined previously. In a typical preparation, IVe is treated with benzaldehyde V to give a compound of Formula IIIe which is then reacted with an alkyl transfer reagent in the presence of a Lewis acid to furnish compound Ie. The typical reaction conditions are similar to those described in Schemes 2 and 3 for compounds of Formula Ia, except that the reaction with benzaldehyde V requires higher temperatures, preferably between 100° C. and about 120° C. When alcohols are used as solvent, analogs of compounds of Formula IIIe wherein the hydroxyl group is replaced with an alkoxy group can also be obtained. For example, with MeOH as solvent one can obtain the methoxy analogs.

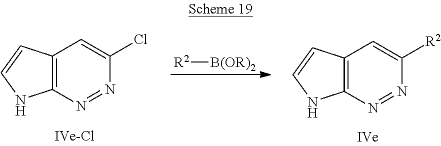

Compounds of Formula IVe can be prepared from IVe-Cl as in Scheme 19, wherein R² is as defined previously and B(OR)₂ is a suitable boronic acid/ester. In a typical preparation of compounds of Formula IVe, the compound of Formula IVe-Cl is reacted with a suitable boronic acid/ester [R²—B(OR)₂] in a suitable solvent via typical Suzuki coupling procedures, applying reaction conditions substantially similar to those described for compounds of Formula Ia. One skilled in the art will appreciate that alternative methods may be applicable for preparing compounds of Formula IVe from IVe-Cl. For example, compound of Formula IVe-Cl could be reacted with a suitable organotin reagent R²—SnBu₃ or the like in a suitable solvent via typical Stille coupling procedures.

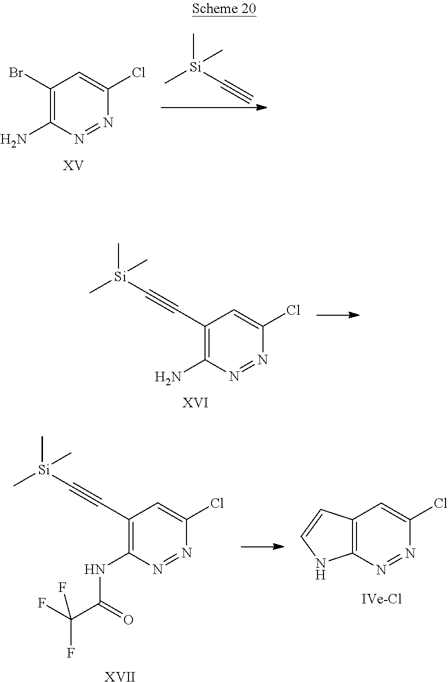

The compound of Formula IVe-Cl may be prepared as in Scheme 20, starting from the known 4-Bromo-6-chloro-pyridazin-3-ylamine (compound XV). Sonogashira coupling of XV with TMS-acetylene using a palladium catalyst and CuI followed by acylation with trifluoroacetic anhydride gives compound XVII, which is subsequently cyclized by heating with CuI in N-methylpyrrolidone.

Compounds of Formula Ie have a chiral center at the carbon atom that connects the pyrrolopyridazine core with X and the phenyl ring substituted with R1. Enantiomerically pure compounds Ie can be prepared by the methods discussed for the compounds of Formula Ia and the general skill in the art.

The building blocks R²-A¹¹ and R²—B(OR)₂ whose use for the preparation of compounds of the present invention is described above may be prepared as follows.

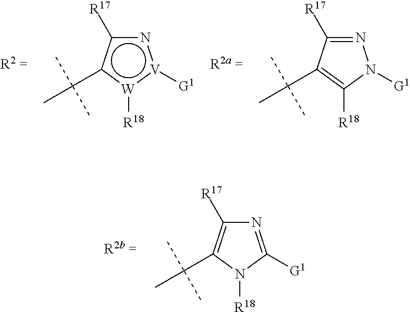

R^{2a}=R² wherein W—V═C—N; R^{2b}=R² wherein W—V═N—C.

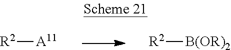

The building block R²—B(OR)₂ may be prepared as in Scheme 21 from the building block R²-A¹¹, wherein R² is as defined previously, A¹¹ is halogen such as Cl, Br, or I, or trifluoromethanesulfonate, and B(OR)₂ is a suitable boronic acid/ester. The conversion may be accomplished by palladium catalysis under conditions similar to those described above in Schemes 4, 11, and 14. An alternate route for compounds R²-A¹¹ wherein A¹¹ is Br or I consists of halogen-metal exchange with organolithium or magnesium reagents followed by reaction with a boron reagent. Suitable reagents for A¹¹=I include, but are not limited to, iPrMgCl, iPrMgBr, or iPrMgCl.LiCl as organomagnesium reagents and MeOB(pinacol) or B(OMe)₃ as boron reagents. Suitable reagents for A¹¹=Br include, but are not limited to, nBuLi as organolithium reagent and MeOB(pinacol) or B(OMe)₃ as boron reagents.

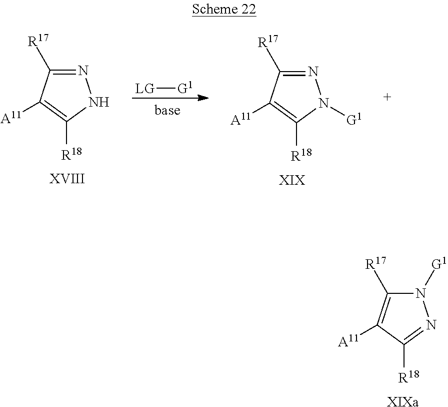

As shown in Scheme 22, building blocks containing R^{2a} a may be prepared by alkylating a pyrazole XVIII that is unsubstituted on the nitrogen atoms with an alkylating agent LG-G¹, wherein LG is a leaving group such as the halogens Cl, Br, and I, or a sulfonate ester such as tosylate, mesylate, or trifluoromethanesulfonate. A¹¹ is halogen such as Cl, Br, or I. If R¹⁷≠R¹⁸, mixtures of regioisomers resulting from alkylation at either of the two nitrogen atoms of the pyrazole may be formed. This reaction can also be conducted with pyrazoles that have a suitable boronic acid/ester B(OR)₂ in place of A¹¹.

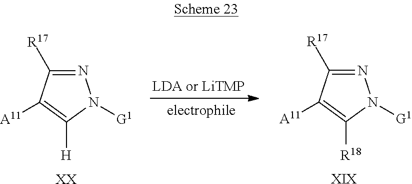

As shown in Scheme 23, building blocks containing R^{2a} of Formula XX that are unsubstituted at C5, i.e., R¹⁸=H, may be selectively functionalized at C5 by deprotonation with a strong base such as LDA or LiTMP in a solvent such as THF followed by reaction with a suitable electrophile. Examples for electrophiles and the resulting substituents R¹⁸ include, but are not limited to, methyl iodide (R¹⁸=methyl), ethyl iodide (R¹⁸=ethyl), C₂Cl₆ (R¹⁸=Cl), N-fluorobenzenesulfonimide (R¹⁸=F), DMF (R¹⁸=CHO), CO₂ (R¹⁸=CO₂H). This reaction can also be conducted with pyrazoles that have a suitable boronic acid/ester B(OR)₂ in place of A¹¹.

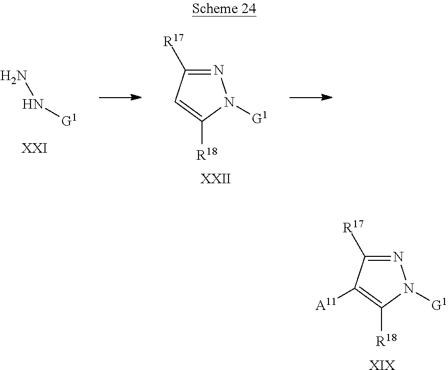

As shown in Scheme 24, the pyrazole ring in building blocks containing R^{2a} of Formula XIX may also be synthesized de novo by condensation of a hydrazine derivative H₂N—NH-G¹ with a 1,3-dicarbonyl-type reagent followed by reaction with a halogenating agent to introduce A¹¹. Examples for halogenating agents include, but are not limited to, pyridinium perbromide or NBS (for A¹¹=Br), NIS or ICI (for A¹¹=I), or NCS (for A¹¹=Cl).

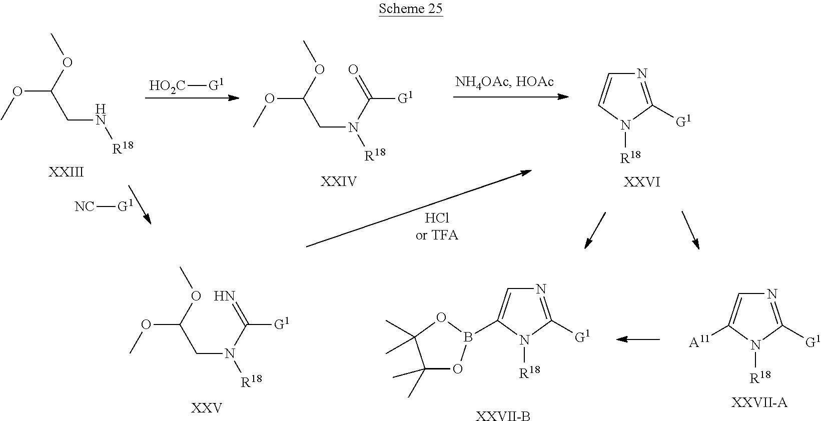

The imidazole ring in building blocks of Formula XXVII-N-B containing R^{2b}, wherein R¹⁸ is H, aliphatic, or cycloalkyl, may be synthesized de novo as shown in Scheme 25. The carboxylic acid HO₂C-G¹ is reacted with an aminoacetaldehyde acetal XXIII under typical conditions for amide formation (e.g., EDCI+HOBt, mixed anhydrides, TBTU) to give an amide, which upon heating with NH₄OAc in acetic acid cyclizes to form the imidazole ring, yielding a compound of Formula XXVI. R¹⁸ in the aminoacetaldehyde acetal XXIII can be H, aliphatic, or cycloalkyl; if R¹⁸=H in XXIII then it is convenient to introduce R¹⁸≠H by alkylation of XXVI with R¹⁸-LG wherein LG is a leaving group such as Cl, Br, I, mesylate, tosylate, or triflate. In an alternate route to XXVI, the aminoacetaldehyde acetal XXIII can be reacted with the nitrile in the presence of CuCl without solvent to obtain the amidine of Formula XXV, which is cyclized with HCl or TFA in alcoholic solvents such as methanol or ethanol to give the imidazole of Formula XXVI (as described in Tetrahedron Letters 2005, 46, 8369-8372). The imidazole XXVI can be halogenated at C5 to give a compound of Formula XXVII-A with a suitable halogenating agent such as NBS (for A¹¹=Br), NIS or ICI (for A¹¹=I), or NCS (for A¹¹=Cl), in solvents such as THF, EtOAc, DCM, DMF, and the like. It can also be borylated at C5 to give a compound of Formula XXVII-B with pinacolborane or bis(pinacolato)diboron in the presence of a catalyst consisting of an iridium complex and a 2,2′-bipyridine. Preferred catalysts include [Ir(OMe)(COD)]₂ and 2,2′-di-tert-butyl-bipyridine.

Building blocks containing R^{2b}, wherein R¹⁷≠H and R¹⁸ is H, aliphatic, or cycloalkyl, may be prepared following the same route but starting from analogs of the acetal XXIII that are substituted at the acetal carbon atom with R¹⁷. Alternatively, the imidazole XXVI can be halogenated at C4 and C5 by using >2 equivalents of halogenating agent, and the imidazole XXVII-A can also be halogenated at C4, resulting in compounds wherein R¹⁷=halogen. Due to the different reactivity of halogens at C5 vs. C4, each position can be modified selectively, allowing the conversion of R¹⁷=halogen to other functionalities as defined above.

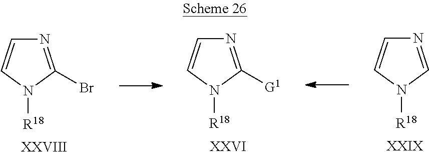

The imidazoles of Formula XXVI may also be prepared from 2-bromoimidazoles XXVIII or imidazoles XXIX as shown in Scheme 26 by a variety of methods depending on the G¹ substituent. For example, the Br in XXVIII may be displaced by nucleophiles or reacted in transition metal-catalyzed reactions. Bromine-lithium exchange generates an anion that can be reacted with electrophiles; the same anion can also be obtained by deprotonating XXIX with a strong base such as LDA, LiTMP, or BuLi.

Further methods of functionalizing and building up the pyrazole and imidazole rings can be found in the general literature, e.g., Volume 3 of Comprehensive Heterocyclic Chemistry II (Pergamon).

The functional groups present in R¹⁷, R¹⁸, and G¹ may be further modified by methods known to someone skilled in the art and the general literature such as the book Comprehensive Organic Transformations by R. C. Larock.

As will be apparent to the skilled artisan, the synthetic routes/sequences can be modified as desired for the preparation of a given compound.

### EXPERIMENTAL

Unless otherwise noted, all materials/reagents were obtained from commercial suppliers and used without further purification. ¹H NMR (400 MHz or 300 MHz) and ¹³C NMR (100.6 MHz) spectra were recorded on Bruker or Varian instruments at ambient temperature with tetramethylsilane or the residual solvent peak as the internal standard. The line positions or multiples are given in ppm (δ) and the coupling constants (J) are given as absolute values in Hertz (Hz). The multiplicities in ¹H NMR spectra are abbreviated as follows: s (singlet), d (doublet), t (triplet), q (quartet), quint (quintet), m (multiplet), m_{c} (centered multiplet), br or broad (broadened), AA′BB′. The signal multiplicities in ¹³C NMR spectra were determined using the DEPT135 pulse sequence and are abbreviated as follows: +(CH or CH₃), −(CH₂), C_{quart}(C). Reactions were monitored by thin layer chromatography (TLC) on silica gel 60 F₂₅₄ (0.2 mm) precoated aluminum foil and visualized using UV light. Flash chromatography was performed with silica gel (400-230 mesh). Preparatory TLC was performed on Whatman LK6F Silica Gel 60 Å size 20×20 cm plates with a thickness of 500 or 1000 μm. Hydromatrix (=diatomaceous earth) was purchased from Varian. Mass-directed HPLC purification of compounds was performed on a Waters system composed of the following: 2767 Sample Manager, 2525 Binary Gradient Module, 600 Controller, 2996 Diode Array Detector, Micromass ZQ2000 for ionization, Phenomenex Luna 5μ C18(2) 100 Å 150×21.2 mm 5μ column with mobile phases of 0.01% Formic Acid Acetonitrile (A) and 0.01% Formic Acid in HPLC water (B), a flow rate of 20 mL/min, and a run time of 13 min. LC-MS data was collected on ZQ2, ZQ3, or UPLC-ACQUITY. ZQ2 is an Agilent 1100 HPLC equipped with a Gilson 215 Liquid Handler, Gilson 819 Injection Module, and Waters Micromass ZQ2000 for ionization. ZQ3 is an Agilent 1100 HPLC equipped with an HP Series 1100 auto injector and Waters Micromass ZQ2000 for ionization. Both systems use the Xterra MS C18, 5μ particle size, 4.6×50 mm with a mobile phase of Acetonitrile (A) and 0.01% Formic Acid in HPLC water (B). The flow rate is 1.3 mL/min, the run time is 5 min, and the gradient profiles are 0.00 min 5% A, 3.00 min 90% A, 3.50 min 90% A, 4.00 min 5% A, 5.00 min 5% A for polar_{—}5 min and 0.00 min 25% A, 3.00 min 99% A, 3.50 min 99% A, 4.00 min 25% A, 5.00 min 25% A for nonpolar_{—}5 min. All Waters Micromass ZQ2000 instruments utilized electrospray ionization in positive (ES+) or negative (ES−) mode. The Waters Micromass ZQ2000 instruments from ZQ2 and ZQ3 can also utilize atmospheric pressure chemical ionization in positive (AP+) or negative (AP−) mode. The Waters UPLC-ACQUITY system consists of an ACQUITY sample manager attached to ACQUITY SQ MS and ACQUITY PDA detectors. It uses an ACQUITY UPLC BEH® C18 2.1×50 mm 1.7 μm column with a mobile phase of 0.1% formic acid in water (A) and 0.1% formic acid in acetonitrile (B). The flow rate is 1.0 mL/min, run time is 2 min, and the gradient profile is 0.00 min 95% A, 1.50 min 1% A, 1.85 min 1% A, 2.0 min 95% A for analytical. UV detection is at 254 nm, and the MS utilizes electrospray ionization in positive mode (ES+). HPLC purification of compounds was performed on a Waters system consisting of a 2767 Sample Manager, 1525EF Binary Pump, and a 2487 Dual λ Absorbance Detector. The system uses Phenomenex Luna C18(2), 5μ particle size, 50×21.2 mm columns with a mobile phase of Acetonitrile/0.25% Formic Acid and HPLC water/0.25% Formic Acid. Alternatively, a Gilson system (“Gilson HPLC”) consisting of a 215 Liquid Handler, 819 Injection Module, a 322 Pump, and a 155 UV/VIS dual wavelength detector set to 254 and 210 nm was used. This system uses Phenomenex Luna C18(2), 5μ particle size, 50×21.2 mm or 60×21.2 mm columns with a mobile phase of Acetonitrile and 0.1% Formic Acid in HPLC water. The flow rate is 15 mL/min and the run time is 25 min. The HPLC system for determination of enantiomeric purity consists of an Agilent 1100 HPLC and Chiralcel or Chiralpak 4.6×150 mm columns (Daicel Chemical Ind., Ltd.), eluting with acetonitrile/water mixtures. All melting points were determined with a MeI-Temp II apparatus and are uncorrected. Elemental analyses were obtained by Atlantic Microlab, Inc., Norcross, Ga. 2,6-Dichloro-3-fluorobenzaldehyde

To a solution of (2,6-Dichloro-3-fluorophenyl)methanol (100 g, 0.51 mol) in dichloromethane (450 mL) was added a solution of sodium bromide (54 g, 0.53 mol, in 90 mL water). The rapidly stirred biphasic mixture was cooled to −7° C. and TEMPO (1.54 g, 0.0100 mol) was added. A solution of 0.81 M sodium hypochlorite (823 mL, 0.66 mol) saturated with sodium bicarbonate (75 g) was added dropwise over a period of 1 h while maintaining the temperature below −2° C. After the addition the reaction mixture was stirred for 30 min. The two layers separated and the DCM layer was washed with aq. solution of sodium thiosulfate. The DCM layer was dried (Na₂SO₄) and concentrated on rotary evaporator without using vacuum (aldehyde is volatile) to give the title compound as a solid, mp. 63-65° C. ¹H NMR (CDCl₃, 300 MHz): δ=7.23 (dd, 1H, J=7.8, 9.0 Hz), 7.35 (dd, 1H, J=4.5, 9.3 Hz), 10.2 (s, 1H).

Alternate preparation: To a solution of 2,4-dichloro-1-fluorobenzene (100 g, 0.606 mol) in THF (1.4 L) under nitrogen at −78° C., was added a 2.5 M solution of n-BuLi in hexanes (267 mL, 0.666 mol) dropwise over a period of 30 min, maintaining the temperature between −70 to −78° C. After 1.5 h stirring at −78° C., methyl formate (72.6 mL, 1.21 mol) was added slowly, and the reaction mixture was stirred overnight, warming up to rt. The reaction was quenched with sat. aqueous NH₄Cl (200 mL) and the organic layer was separated. The organic solvents were removed by distillation at atmosphere pressure and the crude material which contained a small amount of THF was crystallized from hexanes to give the title compound.

### (2,6-Dichloro-3-fluorophenyl)methanol

To a solution of 2,6-Dichloro-3-fluorobenzoic acid (125 g, 0.59 mol) in THF (200 mL) was added BH₃.THF (592 mL, 592 mmol, 1M solution in THF) dropwise at room temperature. The reaction mixture was heated to reflux for 12 h. The borane was quenched with methanol (200 mL) and the resulting solution was concentrated to dryness. The residue was again co-evaporated with methanol to remove most of the trimethylborate. To the residue was added aq. sodium carbonate (50 g in 500 mL). The mixture was cooled and a white fine precipitate was filtered off to give the title compound. ¹H NMR (CDCl₃, 300 MHz): δ=2.10 (t, 1H, J=6.9 Hz), 4.96 (d, 2H, J=6.9 Hz), 7.09 (dd, 1H, J=8.1, 9.0 Hz), 7.29 (dd, 1H, J=4.8, 9.0 Hz).

### 2,6-Dichloro-3-fluorobenzoic acid

To a cooled (−5° C.) solution of sodium hydroxide (252 g, 6.3 mol) in water (800 mL) was added bromine (86 mL, 1.68 mol) dropwise. The temperature of the reaction mixture was kept below 5° C. during the addition. A solution of 1-(2,6-Dichloro-3-fluorophenyl)ethanone (100 g, 480 mmol) in dioxane (800 ml) was added to the solution of sodium hypobromide in 1 h while maintaining the temperature below 0° C. The reaction mixture was warmed to room temperature and stirred for 2 h. After the TLC showed absence of starting material, the excess sodium hypobromide was destroyed with sodium sulfite (100 g in 100 mL water). The resulting solution was heated to 90° C. for 2 h. The reaction mixture was acidified with conc. HCl with vigorous stirring. The acidic solution was concentrated to remove all the dioxane and then extracted with dichloromethane (2×500 mL). The organic layer was dried (Na₂SO₄) and concentrated to give an oily residue, which after trituration with hexanes gave the title compound as a white solid. ¹H NMR (CDCl₃, 300 MHz): δ=7.20 (dd, 1H, J=8.7, 8.4 Hz), 7.33 (dd, 1H, J=9.3, 4.5 Hz).

### 4-[4-(4,4,5,5-Tetramethyl[1,3,2]dioxaborolan-2-yl)pyrazol-1-yl]piperidine hydrochloride

To a solution of 4-[4-(4,4,5,5-Tetramethyl-1,3,2-dioxaborolan-2-yl)pyrazol-1-yl]-piperidine-1-carboxylic acid tert-butyl ester (3.02 g, 8.00 mmol) in 1,4-dioxane (30 mL, 400 mmol), 4.0 M of HCl in 1,4-Dioxane (30 mL) was added and the reaction was stirred at 35° C. for 3 h. The reaction mixture was concentrated in vacuo to a white solid. The material was slightly hygroscopic. All free-flowing material was transferred to a vial and dried under vacuum for several hours. The material thus obtained was used in further reactions without purification. ¹H NMR (400 MHz, CDCl₃): δ=1.33 (s, 12H), 2.49 (br s, 4H), 3.18 (br s, 2H), 3.59-3.70 (m, 2H), 4.71 (br s, 1H), 7.87 (s, 2H), 9.84 (br s, 2H). MS (ES+): m/z 278.11 (100) [MH⁺]. HPLC: t_{R}=1.99 min (ZQ3, polar_{—}5 min).

### 4-[4-(4,4,5,5-Tetramethyl-1,3,2-dioxaborolan-2-yl)pyrazol-1-yl]-piperidine-1-carboxylic acid tert-butyl ester

A mixture of 4-(4,4,5,5-tetramethyl[1,3,2]dioxaborolan-2-yl)-1H-pyrazole (30.0 g, 154 mmol), 4-methanesulfonyloxypiperidine-1-carboxylic acid tert-butyl ester (52.5 g, 200 mmol) and cesium carbonate (80.1 g, 246 mmol) in anhydrous DMF (400 mL) was heated to 100° C. for 24 h. DMF was removed under high vacuum. The residue was then diluted with water (200 mL) and extracted with EtOAc (3×200 mL). The combined organic phases were washed with water (3×50 mL) and brine (100 mL), dried over anhydrous sodium sulfate, filtered and concentrated under reduced pressure. To the orange-brown oily residue was added diisopropyl ether (300 mL), and the mixture was stirred at 0° C. for 2 h. Colorless crystals separated out that were filtered off and dried in vacuo to give a 1^{st} crop of the title compound. The filtrate was then concentrated in vacuo, the residue was mixed with diisopropyl ether (100 mL), a small amount of the 1^{st} crop was added as a seed, and the mixture was stirred overnight. The resulting white precipitate was filtered and dried in vacuo as 2^{nd} crop of the title compound. ¹H NMR (300 MHz, CDCl₃): δ=1.33 (s, 12H), 1.48 (s, 9H), 1.85-1.93 (m, 2H), 2.15-2.18 (m, 2H), 2.83-2.92 (m, 2H), 4.23-4.39 (m, 3H), 7.76 (s, 1H), 7.84 (s, 1H).

### 4-Methanesulfonyloxypiperidine-1-carboxylic acid tert-butylester

To a solution of 1-Boc-4-hydroxypiperidine (32.2 g, 0.160 mol) in DCM (400 mL) were added triethylamine (26.8 mL, 0.192 mol), methanesulfonyl chloride (13.6 mL, 0.176 mol) and 4-dimethylaminopyridine (0.20 g, 0.0016 mol) at 0° C. under nitrogen atmosphere. The resulting mixture was slowly warmed to rt and stirred at rt overnight. The mixture was washed with sat. aq. NaHCO₃ (3×80 mL), brine (2×80 mL), and dried over anhydrous sodium sulfate. The filtrate was concentrated to give the title compound as a white solid. It was used in the next step without further purification. ¹H NMR (400 MHz, CDCl₃): δ=1.47 (s, 9H), 1.80-1.85 (m, 2H), 1.95-1.99 (m, 2H), 3.05 (s, 3H), 3.28-3.34 (m, 2H), 3.68-3.74 (m, 2H), 4.89 (m_{c}, 1H).

### Example 1

### 7-[(2,6-dichloro-3-fluorophenyl)methoxymethyl]-2-(1-piperidin-4-yl-1H-pyrazol-4-yl)-5H-pyrrolo[2,3-b]pyrazine

To a solution of 4-(4-{7-[(2,6-dichloro-3-fluorophenyl)methoxymethyl]-5H-pyrrolo[2,3-b]pyrazin-2-yl}-pyrazol-1-yl)-piperidine-1-carboxylic acid tert-butyl ester (0.042 g, 0.07 mmol) in DCM (2 mL) at RT under nitrogen was added 2.0 M HCl in ether (2.0 mL, 4.0 mmol). The mixture was stirred for 16 h. Evaporation of solvents under reduced pressure gave the title compound as dihydrochloride salt. ¹H NMR (CD₃OD, 300 MHz): δ=2.40-2.51 (m, 4H), 3.37 (s, 3H), 3.66-3.70 (m, 2H), 4.80-4.90 (m, 3H), 6.80 (s, 1H), 7.39-7.46 (m, 1H), 7.56-7.62 (m, 1H), 7.82 (s, 1H), 8.30 (s, 1H), 8.69 (s, 1H), 8.99 (s, 1H).

### 4-(4-{7-[(2,6-Dichloro-3-fluorophenyl)methoxymethyl]-5H-pyrrolo[2,3-b]pyrazin-2-yl}-pyrazol-1-yl)-piperidine-1-carboxylic acid tert-butyl ester

To a degassed solution of 2-bromo-7-[(2,6-dichloro-3-fluorophenyl)methoxymethyl]-5H-pyrrolo[2,3-b]pyrazine (0.150 g, 0.38 mmol) in DME/H₂O (12.5 mL, 4:1) were added 4-[4-(4,4,5,5-tetramethyl-[1,3,2]dioxaborolan-2-yl)-pyrazol-1-yl]-piperidine-1-carboxylic acid tert-butyl ester (0.209 g, 0.55 mmol), PdCl₂(PPh₃)₂ (21 mg, 5 mol %) and Na₂CO₃ (0.157 g, 1.48 mmol). The mixture was heated at 80° C. for 16 h, cooled to RT, and concentrated in vacuo. To the residue was added water (30 mL), and the mixture was extracted with ethyl acetate (3×20 mL). The combined organic layers were washed with water (20 mL), dried over anhydrous Na₂SO₄, filtered, and concentrated in vacuo. The crude product was purified by column chromatography on silica gel using EtOAc/DCM (1:1) to give the title compound as a solid. ¹H NMR (CDCl₃, 300 MHz): δ=1.56 (s, 9H), 2.04-2.84 (m, 2H), 2.37-2.76 (m, 2H), 3.00 (t, J=12 Hz, 2H), 3.58 (s, 3H), 4.33-4.42 (m, 3H), 6.72 (s, 1H), 7.14 (t, J=9.0 Hz, 1H), 7.33-7.39 (m, 2H), 7.44 (d, J=1.5 Hz, 1H), 7.98 (s, 1H), 8.02 (s, 1H), 8.48 (s, 1H), 9.65 (brs, 1H). MS (ES+): m/z=575 (100) [MH⁺].

### 2-Bromo-7-[(2,6-dichloro-3-fluorophenyl)methoxymethyl]-5H-pyrrolo[2,3-b]pyrazine and 2-Bromo-5H-pyrrolo[2,3-b]pyrazin-7-yl-(2,6-dichloro-3-fluorophenyl)methanol

A mixture of 2,6-dichloro-3-fluorobenzaldehyde (2.11 g, 11 mmol), 2-bromo-5H-pyrrolo[2,3-b]pyrazine (2, 1.98 g, 10 mmol) and KOH (0.84 g, 15 mmol) in methanol (25 mL) was stirred at RT for 16 h. The reaction mixture was evaporated to dryness and to the residue was added water (60 mL). The mixture was extracted with ethyl acetate (3×40 mL) and the combined organic layers were washed with brine (20 mL), dried over anhydrous Na₂SO₄, filtered, and concentrated in vacuo. The residue was purified by column chromatography on silica gel using EtOAc/DCM (1:9) to give the methoxy and the hydroxy derivatives as solids. 2-Bromo-7-[(2,6-dichloro-3-fluorophenyl)methoxymethyl]-5H-pyrrolo[2,3-b]pyrazine: ¹H NMR (DMSO-d₆, 300 MHz): δ=3.40 (s, 3H), 6.42 (s, 1H), 7.46-7.60 (m, 2H), 7.82 (s, 1H), 8.40 (s, 1H). 2-Bromo-5H-pyrrolo[2,3-b]pyrazin-7-yl-(2,6-dichloro-3-fluorophenyl)methanol: ¹H NMR (DMSO-d₆, 300 MHz): δ=6.21 (d, J=4.8 Hz, 1H), 6.73 (d, J=5.1 Hz, 1H), 7.35-7.50 (m, 2H), 7.79 (s, 1H), 8.34 (s, 1H), 12.15 (brs, 1H).

### Example 2

### 7-[1-(2,6-dichloro-3-fluorophenyl)ethyl]-2-(1-piperidin-4-yl-1H-pyrazol-4-yl)-5H-pyrrolo[2,3-b]pyrazine

To a solution of 4-(4-{7-[1-(2,6-dichloro-3-fluorophenyl)ethyl]-5H-pyrrolo[2,3-b]pyrazin-2-yl}-pyrazol-1-yl)-piperidine-1-carboxylic acid tert-butyl ester (0.105 g, 0.19 mmol) in CH₂Cl₂ (5 mL) at RT under nitrogen was added 2.0 M HCl in ether (2 mL, 4 mmol). The mixture was stirred for 16 h. Evaporation of solvents under reduced pressure gave the title compound as dihydrochloride salt. ¹H NMR (CD₃OD, 300 MHz): δ=2.01 (d, J=7.2 Hz, 3H), 2.45 (brs, 4H), 3.66 (brs, 2H), 4.74 (brs, 2H), 5.50-5.18 (m, 1H), 7.27 (t, J=12 Hz, 1H), 7.48-7.52 (m, 1H), 7.96 (1H), 8.12 (s, 1H), 8.47 (s, 1H), 8.79 (s, 1H).

### 4-(4-{7-[1-(2,6-Dichloro-3-fluorophenyl)ethyl]-5H-pyrrolo[2,3-b]pyrazin-2-yl}-pyrazol-1-yl)-piperidine-1-carboxylic acid tert-butyl ester

To a degassed solution of 2-bromo-7-[1-(2,6-dichloro-3-fluorophenyl)ethyl]-5H-pyrrolo[2,3-b]pyrazine (0.350 g, 0.90 mmol) in DME/H₂O (12.5 mL, 4:1) were added 4-[4-(4,4,5,5-tetramethyl-[1,3,2]dioxaborolan-2-yl)-pyrazol-1-yl]-piperidine-1-carboxylic acid tert-butyl ester (0.388 g, 1.03 mmol), PdCl₂(PPh₃)₂ (0.74 g, 10 mol %) and Na₂CO₃ (0.318 g, 3.0 mmol), and the mixture was heated at 80° C. for 16 h. After cooling to RT, the reaction mixture was concentrated in vacuo. To the residue was added water (40 mL), and the mixture was extracted with ethyl acetate (3×30 mL). The combined organic layer were washed with water (30 mL), dried over anhydrous Na₂SO₄, filtered, and concentrated in vacuo. The crude product was purified by column chromatography on silica gel using EtOAc/DCM (1:1) to give the title compound as a solid. ¹H NMR (CDCl₃, 300 MHz): δ=1.54 (s, 9H), 2.01 (d, J=7.2 Hz, 3H); 2.03-2.07 (m, 2H); 2.22-2.30 (m, 2H); 3.00 (t, J=12 Hz, 2H); 4.32-4.40 (m, 3H); 5.49 (q, J=7.2 Hz, 1H); 7.02-7.08 (m, 1H); 7.49-7.50 (m, 1H); 7.71-7.80 (m, 1H); 7.91 (s, 1H); 7.97 (s, 1H); 8.45 (s, 1H); 9.02 (brs, 1H).

### 2-Bromo-7-[1-(2,6-dichloro-3-fluorophenyl)ethyl]-5H-pyrrolo[2,3-b]pyrazine

A solution of 2-bromo-5H-pyrrolo[2,3-b]pyrazin-7-yl-(2,6-dichloro-3-fluorophenyl)methanol (0.390 g, 1.0 mmol) in THF (10 mL) was cooled to −40° C. under nitrogen. To this solution were added dropwise BF₃.OEt₂ (1.25 mL, 10 mmol) followed by dimethyl zinc (2M in toluene, 5.0 mL, 10 mmol) (Note: Pyrophoric). The reaction mixture was allowed to warm to RT over 30 min and then heated at 60° C. for 16 h. The reaction mixture was cooled to −40° C., and an aq. satd. solution of NH₄Cl (5 mL) was added slowly. The reaction mixture was concentrated in vacuo. Water (50 mL) was added to the residue, and the mixture was extracted with ethyl acetate (3×30 mL). The combined organic layers were washed with water (20 mL), dried over anhydrous Na₂SO₄, and concentrated in vacuo. The residue was purified by column chromatography on silica gel using EtOAc/DCM (1:9) to give the title compound as a solid. ¹H NMR (CDCl₃, 300 MHz): δ=1.90 (d, J=7.2 Hz, 3H), 5.42 (q, J=7.2 Hz, 1H), 6.92-7.02 (m, 1H), 7.29-7.35 (m, 1H), 7.39 (s, 1H), 8.21 (s, 1H), 8.88 (brs, 1H).

### Example 3

### 4-(4-{7-[1-(2,6-dichloro-3-fluorophenyl)ethyl]-5H-pyrrolo[2,3-b]pyrazin-2-yl}-1H-pyrazol-1-yl)piperidine-1-carbaldehyde

A mixture of 7-[1-(2,6-dichloro-3-fluorophenyl)ethyl]-2-[1-(piperidin-4-yl)-1H-pyrazol-4-yl]-5H-pyrrolo[2,3-b]pyrazine (35.0 mg, 0.0658 mmol), formic acid (6.04 mg, 0.131 mmol), TBTU (42.1 mg, 0.131 mmol), DIPEA (0.06 mL, 0.3 mmol) and DCM (4 mL, 70 mmol) was stirred at rt for 30 min. The solution was transferred to a separatory funnel and extracted with DCM and water. The organic layer was dry-loaded onto silica gel for column chromatography, eluting with 1-3% (7N NH₃ in MeOH)/DCM. The fractions containing the pure product were concentrated in vacuo, and redissolved in DCM. 2 M of HCl in Et₂O (0.5 mL, 1 mmol) was added, and the mixture was stirred at rt for 20 min. The material was concentrated in vacuo to afford the title compound as hydrochloride salt as a white solid. ¹H NMR (400 MHz, CD₃OD): δ=1.93 (d, J=7.3 Hz, 3H), 1.94-2.08 (m, 2H), 2.15-2.34 (m, 2H), 2.94 (t, J=15.7 Hz, 1H), 3.92 (d, J=12.9 Hz, 1H), 4.42-4.60 (m, 2H), 5.42 (q, J=7.4 Hz, 1H), 7.16 (t, J=8.6 Hz, 1H), 7.31-7.43 (m, 1H), 7.71 (d, J=1.3 Hz, 1H), 7.95 (s, 1H), 8.10 (br. s., 1H), 8.19 (s, 1H), 8.52 (s, 1H). MS (ES+): m/z=487.06/489.04 (100/75) [MH⁺]. HPLC: t_{R}=3.32 min (polar_{—}5 min, ZQ3).

### Example 4

### 1-[(1R)-1-(2,6-dichloro-3-fluorophenyl)ethyl]-6-(1-methyl-1H-pyrazol-4-yl)-1H-pyrrolo[3,2-b]pyridine

A mixture of 6-bromo-1-[(1R)-1-(2,6-dichloro-3-fluorophenyl)ethyl]-1H-pyrrolo[3,2-b]pyridine (10.0 mg, 0.0258 mmol), 1-methyl-4-(4,4,5,5-tetramethyl-1,3,2-dioxaborolan-2-yl)-1H-pyrazole (0.0107 g, 0.0515 mmol), Pd(PPh₃)₄ (3 mg, 0.002 mmol), potassium carbonate (10.7 mg, 0.0773 mmol) and 4:1 dioxane:water (0.7 mL) was heated in a microwave reactor at 95° C. for 20 min. The solution was used directly for HPLC purification. The fractions containing the pure product were concentrated in vacuo to afford the title compound as a white solid. ¹H NMR (400 MHz, CD₃OD): δ=2.12 (d, J=7.1 Hz, 3H), 3.91 (s, 3H), 6.42 (q, J=7.1 Hz, 1H), 6.66 (d, J=3.3 Hz, 1H), 7.23-7.29 (m, 1H), 7.35 (s, 1H), 7.48 (dd, J=9.0, 4.9 Hz, 1H), 7.61 (s, 1H), 7.84 (s, 1H), 7.99 (d, J=3.5 Hz, 1H), 8.50 (br. s., 1H). MS (ES+): m/z=388.97/390.97 (100/75) [MH⁺]. HPLC: t_{R}=2.55 min (polar_{—}5 min, ZQ3).

### 6-Bromo-1-[(1R)-1-(2,6-dichloro-3-fluorophenyl)ethyl]-1H-pyrrolo[3,2-b]pyridine

To a solution of 6-bromo-1H-pyrrolo[3,2-b]pyridine (100.0 mg, 0.5075 mmol) in dimethyl sulfoxide (2.0 mL, 30 mmol) was added sodium hydride (10.0 mg, 0.417 mmol) at rt, and stirred until bubbling stopped. (1S)-1-(2,6-Dichloro-3-fluorophenyl)ethyl methanesulfonate (110.0 mg, 0.382 mmol) was then added, and the mixture was allowed to stir at rt overnight. The material was transferred to a separatory funnel, dissolved in EtOAc, and washed with water (3×). The organic layer was dry-loaded onto silica gel for column chromatography, eluting with 10-30% EtOAc/hexanes. The fractions containing the pure product were concentrated in vacuo to afford the title compound as a thick, clear gel. ¹H NMR (400 MHz, CD₃OD): δ=2.10 (d, J=7.3 Hz, 3H), 6.38 (q, J=7.2 Hz, 1H), 6.68 (dd, J=3.5, 1.0 Hz, 1H), 7.26-7.32 (m, 1H), 7.35-7.39 (m, 1H), 7.48 (dd, J=9.0, 4.9 Hz, 1H), 8.01 (d, J=3.5 Hz, 1H), 8.33 (d, J=2.0 Hz, 1H). MS (ES+): m/z=386.86/388.83/390.87 (75/100/75) [MH⁺]. HPLC: t_{R}=3.89 min (polar_{—}5 min, ZQ3).

### (1S)-1-(2,6-Dichloro-3-fluorophenyl)ethyl methanesulfonate

To a cooled (ice bath) solution of (S)-1-(2,6-Dichloro-3-fluorophenyl)ethanol (4.00 g, 16.3 mmol) and triethylamine (3.4 mL, 24 mmol) in toluene (20 mL) was added dropwise methanesulfonyl chloride (1.64 mL, 21.1 mmol). A white suspension formed that was stirred at 0-5° C. for 35 min. The reaction mixture was diluted with H₂O (20 mL), the layers were separated, and the aqueous layer was extracted with toluene (10 mL). The combined organic layers were washed with water (2×10 mL) and concentrated under vacuum at 40-45° C. to give the title compound as colorless oil containing ≈0.2 eq. of toluene according to ¹H NMR. This material was used directly in the next step. ¹H NMR (CDCl₃, 400 MHz): δ=7.33 (dd, J=9.0, 4.9 Hz, 1H), 7.12 (dd, J=9.0, 8.0 Hz, 1H), 6.45 (q, J=6.8 Hz, 1H), 2.91 (s, 3H), 1.84 (d, J=6.8 Hz).

### Example 5

### 1-[(1R)-1-(2,6-Dichloro-3-fluorophenyl)ethyl]-6-[1-(piperidin-4-yl)-1H-pyrazol-4-yl]-1H-pyrrolo[3,2-b]pyridine

Prepared following the procedure described for previous example, using 4-[4-(4,4,5,5-tetramethyl[1,3,2]dioxaborolan-2-yl)pyrazol-1-yl]piperidine hydrochloride in place of 1-methyl-4-(4,4,5,5-tetramethyl-1,3,2-dioxaborolan-2-yl)-1H-pyrazole. ¹H NMR (400 MHz, CD₃OD): δ=2.14 (d, J=7.1 Hz, 3H), 2.22-2.39 (m, 4H), 3.16-3.27 (m, 2H), 3.58 (ddd, J=13.3, 3.5, 3.4 Hz, 2H), 4.58 (dt, J=10.1, 5.1 Hz, 1H), 6.45 (q, J=7.2 Hz, 1H), 6.67 (d, J=2.8 Hz, 1H), 7.24-7.31 (m, 1H), 7.38 (s, 1H), 7.49 (dd, J=9.0, 4.9 Hz, 1H), 7.69 (s, 1H), 7.97-8.03 (m, 2H), 8.51 (br. s., 1H). MS (ES+): m/z=457.93/459.94 (100/75) [MH⁺]. HPLC: t_{R}=2.17 min (polar_{—}5 min, ZQ3).

### Example 6

### 4-(4-{1-[(1R)-1-(2,6-Dichloro-3-fluorophenyl)ethyl]-1H-pyrrolo[3,2-b]pyridin-6-yl}-1H-pyrazol-1-yl)piperidine-1-carbaldehyde

Prepared following the procedure described for previous example, using 4-[4-(4,4,5,5-tetramethyl-1,3,2-dioxaborolan-2-yl)-1H-pyrazol-1-yl]piperidine-1-carbaldehyde in place of 1-methyl-4-(4,4,5,5-tetramethyl-1,3,2-dioxaborolan-2-yl)-1H-pyrazole. ¹H NMR (400 MHz, CD₃OD): δ=1.86-2.06 (m, 2H), 2.13 (d, J=7.1 Hz, 3H), 2.14-2.26 (m, 2H), 2.90 (td, J=12.9, 3.0 Hz, 1H), 3.32-3.38 (m, 1H), 3.89 (ddd, J=13.5, 2.1, 2.0 Hz, 1H), 4.42-4.56 (m, 2H), 6.44 (q, J=7.1 Hz, 1H), 6.67 (d, J=3.5 Hz, 1H), 7.27 (t, J=8.6 Hz, 1H), 7.39 (s, 1H), 7.49 (dd, J=9.0, 4.9 Hz, 1H), 7.64 (s, 1H), 7.98-8.01 (m, 2H), 8.07 (s, 1H), 8.52 (br. s., 1H). MS (ES+): m/z=485.97/487.98 (100/75) [MH⁺]. HPLC: t_{R}=2.52 min (polar_{—}5 min, ZQ3).

### 4-[4-(4,4,5,5-Tetramethyl-1,3,2-dioxaborolan-2-yl)-1H-pyrazol-1-yl]piperidine-1-carbaldehyde

To a suspension of 4-[4-(4,4,5,5-Tetramethyl-[1,3,2]dioxaborolan-2-yl)-pyrazol-1-yl]-piperidine hydrochloride (202.5 mg, 0.6457 mmol), N-(3-Dimethylaminopropyl)-W-ethylcarbodiimide hydrochloride (204.9 mg, 1.069 mmol), and 4-Dimethylaminopyridine (41.9 mg, 0.343 mmol) in DCM (5 mL, 80 mmol), DIPEA (0.6 mL, 3 mmol) was added at rt; upon addition, all solid went into solution. To this solution, Formic acid (60.0 μL, 1.59 mmol) was added and the reaction was allowed to stir at ambient temperature for 5.5 h. The crude reaction was diluted with DCM and washed with NaHCO₃ (1×). The aqueous layer was extracted with DCM (3×), after which all organic layers were combined, washed with brine (1×), dried over anhydrous Na₂SO₄, filtered, and concentrated in vacuo. MS (ES+): m/z=305.18/306.20/307.20 (50/100/38) [MH⁺]. HPLC: t_{R}=2.74 min (polar_{—}5 min, ZQ3).

### Example 7

### trans-4-(4-{1-[(1R)-1-(2,6-Dichloro-3-fluorophenyl)ethyl]-1H-pyrrolo[3,2-b]pyridin-6-yl}-1H-pyrazol-1-yl)cyclohexanol

A mixture of 6-bromo-1-[(1R)-1-(2,6-dichloro-3-fluorophenyl)ethyl]-1H-pyrrolo[3,2-b]pyridine (10.0 mg, 0.0258 mmol), 1-(trans-4-{[tert-butyl(dimethyl)silyl]oxy}cyclohexyl)-4-(4,4,5,5-tetramethyl-1,3,2-dioxaborolan-2-yl)-1H-pyrazole (0.0209 g, 0.0515 mmol), Pd(PPh₃)₄ (3 mg, 0.002 mmol), potassium carbonate (10.7 mg, 0.0773 mmol) and 4:1 dioxane:water (0.7 mL) was heated in a microwave reactor at 95° C. for 20 min. 2 M of HCl in H₂O (0.3 mL, 0.6 mmol) was added, and the mixture was stirred at rt overnight. The material was passed through a syringe filter pad, and prepared for HPLC purification. The fractions containing the pure product were concentrated in vacuo to afford the title compound as a white solid. ¹H NMR (400 MHz, CD₃OD): δ=1.41-1.54 (m, 2H), 1.83-1.96 (m, 2H), 2.03-2.17 (m, 7H), 3.61-3.71 (m, 1H), 4.13-4.23 (m, 1H), 6.43 (q, J=7.1 Hz, 1H), 6.66 (d, J=3.5 Hz, 1H), 7.26 (t, J=8.6 Hz, 1H), 7.38 (s, 1H), 7.48 (dd, J=9.0, 4.9 Hz, 1H), 7.60 (s, 1H), 7.94 (s, 1H), 7.99 (d, J=3.5 Hz, 1H), 8.50 (br. s., 1H). MS (ES+): m/z=472.97/474.98 (100/75) [MH⁺]. HPLC: t_{R}=2.55 min (polar_{—}5 min, ZQ3).

### 1-(trans-4-{[tert-Butyl(dimethyl)silyl]oxy}cyclohexyl)-4-(4,4,5,5-tetramethyl-1,3,2-dioxaborolan-2-yl)-1H-pyrazole

To a solution of 1-(trans-4-{[tert-butyl(dimethyl)silyl]oxy}cyclohexyl)-4-iodo-1H-pyrazole (500 mg, 1.23 mmol) in THF (10 mL, 100 mmol) at rt was added 1.3 M of isopropylmagnesium chloride in THF (2.8 mL, 3.7 mmol), and the mixture was stirred for 1 h. The reaction was quenched with 2-methoxy-4,4,5,5-tetramethyl-1,3,2-dioxaborolane (0.81 mL, 4.9 mmol), and allowed to stir at rt for 1 h. Sat. NH₄Cl was added, and the organic solvent was removed in vacuo. The material was extracted with DCM and water. The organic layer was concentrated in vacuo to afford the title compound as an oil. MS (ES+): m/z=407.27 (100) [MH⁺]. HPLC: t_{R}=3.28 min (v.v. non-polar_{—}5 min, ZQ3).

### 1-(trans-4-{[tert-Butyl(dimethyl)silyl]oxy}cyclohexyl)-4-iodo-1H-pyrazole

A mixture of trans-4-(4-iodo-1H-pyrazol-1-yl)cyclohexanol (1.00 g, 3.42 mmol), tert-butyldimethylsilyl chloride (1.03 g, 6.85 mmol), 4-dimethylaminopyridine (80 mg, 0.7 mmol), 1H-imidazole (699 mg, 10.3 mmol) and DCM (20 mL, 300 mmol) was stirred rt for 20 min. The material was transferred to a separatory funnel, extracting with DCM and sat. NaHCO₃. The organic layer was dry-loaded onto silica gel for column chromatography, eluting with 3% EtOAc/hexanes. The fractions containing the pure product were concentrated in vacuo to afford the title compound as a clear oil. ¹H NMR (400 MHz, DMSO-d₆): δ=0.05 (s, 6H), 0.86 (s, 9H), 1.33-1.47 (m, 2H), 1.70-1.91 (m, 4H), 1.96 (d, J=11.9 Hz, 2H), 3.58-3.75 (m, 1H), 4.11-4.21 (m, 1H), 7.49 (s, 1H), 7.92 (s, 1H). MS (ES+): m/z=407.05 (100) [MH⁺]. HPLC: t_{R}=3.22 min (v.v. non-polar_{—}5 min, ZQ3).

### Trans- and cis-4-(4-Iodopyrazol-1-yl)cyclohexanol

Sodium borohydride (0.29 g, 7.6 mmol) was added into the EtOH (20 mL) solution of 4-(4-iodopyrazol-1-yl)cyclohexanone (4.5 g, 15.5 mmol) at RT under an atmosphere of nitrogen. The mixture was stirred at RT for 2 h. Work-up: Solvent was evaporated and added water to the residue and extracted with EtOAc (3×60 mL). The combined organic extracts were dried over Na₂SO₄, filtered, and concentrated in vacuo to give an off-white solid. This material was purified by column chromatography on silica gel by eluting with 40 EtOAc/hexanes. The first (less polar) spot obtained was identified as cis isomer and the second (more polar) spot obtained was identified as trans isomer. Cis-isomer: ¹H NMR (300 MHz, CDCl₃): δ=1.63-1.74 (m, 4H), 1.87-1.96 (m, 4H), 2.09-2.19 (m, 2H), 4.07-4.20 (m, 2H), 7.50 (s, 2H). Trans-isomer: colorless solid, mp. 82-86° C. ¹H NMR (400 MHz, CDCl₃): δ=1.42-1.51 (m, 2H), 1.79 (brs, 1H), 1.77-1.99 (m, 2H), 2.09-2.22 (m, 4H), 3.74 (br.tt, J=10.8, 4.0 Hz, 1H), 4.13 (tt, J=11.6, 3.8 Hz. 1H), 7.44 (d, J=0.4 Hz, 1H), 7.50 (d, J=0.4 Hz, 1H). MS (ES+): m/z=293.11 [MH⁺]. HPLC: t_{R}=2.58 min (polar_{—}5 min, ZQ3).

### 4-(4-Iodopyrazol-1-yl)cyclohexanone

A mixture of 1-(1,4-dioxaspiro[4.5]dec-8-yl)-4-iodo-1H-pyrazole (3.0 g, 8.9 mmol), pyridinium p-toluenesulfonate (4.5 g, 17.9 mmol), acetone (100 mL) and H₂O (100 mL) was heated at 60° C. overnight. Work-up: Solvent was evaporated and the residue was extracted with EtOAc (3×60 mL). The combined extracts were washed with water (3×50 mL), brine (50 mL), dried over Na₂SO₄, filtered, and concentrated in vacuo to give the title compound as white solid. It was used in the next step without further purification. ¹H NMR (400 MHz, CDCl₃): δ=2.23-2.63 (m, 8H), 4.57-4.64 (m, 1H), 7.51 (s, 1H), 7.54 (s, 1H). MS (ES+): m/z=291.09 (100). HPLC: t_{R}=2.79 min (polar_{—}5 min, ZQ3).

### 1-(1,4-Dioxaspiro[4.5]dec-8-yl)-4-iodo-1H-pyrazole

A mixture of 1,4-dioxaspiro[4.5]dec-8-yl 4-methylbenzenesulfonate (prepared according to U.S. Pat. No. 4,360,531) (2.0 g, 6.4 mmol), 4-iodopyrazole (1.36 g, 7.0 mmol), K₂CO₃ (1.06 g, 7.7 mmol), and 18-crown-6 (0.2 g, 0.7 mmol) in DMF (5 mL) was heated under nitrogen at 50° C. for 16 h. Water (50 mL) was added to the reaction mixture, which was then extracted with EtOAc (3×40 mL). The combined EtOAc extracts were washed with water (30 mL), dried over Na₂SO₄, filtered, and concentrated in vacuo. The residue was purified by column chromatography on silica gel using EtOAc/CH₂Cl₂ (1:9) to give the title compound. ¹H NMR (CDCl₃, 400 MHz): δ=1.67-1.76 (m, 2H), 1.84-1.91 (m, 2H), 1.99-2.17 (m, 4H), 3.95-3.99 (m, 4H), 4.18-4.27 (m, 1H). MS (ES+): m/z=334.96 (100) [MH⁺]. HPLC: t_{R}=3.26 min (polar_{—}5 min, ZQ3).

### Example 8

### (2S)-3-(4-{1-[(1R)-1-(2,6-Dichloro-3-fluorophenyl)ethyl]-1H-pyrrolo[3,2-b]pyridin-6-yl}-1H-pyrazol-1-yl)propane-1,2-diol

Prepared following the procedure described for Example 7, using 1-{[(4S)-2,2-Dimethyl-1,3-dioxolan-4-yl]methyl}-4-(4,4,5,5-tetramethyl-1,3,2-dioxaborolan-2-yl)-1H-pyrazole. ¹H NMR (400 MHz, CD₃OD): δ=2.13 (d, J=7.1 Hz, 3H), 3.52 (d, J=5.3 Hz, 2H), 3.94-4.03 (m, 1H), 4.15 (dd, J=14.1, 7.6 Hz, 1H), 4.33 (dd, J=13.9, 4.0 Hz, 1H), 6.45 (q, J=7.1 Hz, 1H), 6.68 (d, J=3.5 Hz, 1H), 7.27 (t, J=8.6 Hz, 1H), 7.39-7.43 (m, 1H), 7.49 (dd, J=9.1, 4.8 Hz, 1H), 7.66 (s, 1H), 7.92 (s, 1H), 8.03 (d, J=3.5 Hz, 1H), 8.48-8.57 (m, 1H). MS (ES+): m/z=448.93/450.94 (100/75) [MH⁺]. HPLC: t_{R}=2.36 min (polar_{—}5 min, ZQ3).

### 1-{[(4S)-2,2-Dimethyl-1,3-dioxolan-4-yl]methyl}-4-(4,4,5,5-tetramethyl-1,3,2-dioxaborolan-2-yl)-1H-pyrazole

A solution of 4-(4,4,5,5-Tetramethyl[1,3,2]dioxaborolan-2-yl)-1H-pyrazole (9.24 g, 47.6 mmol), (R)-(−)-(2,2-Dimethyl-1,3-dioxolan-4-yl)methyl p-toluenesulfonate (15.00 g, 52.38 mmol) and CsHCO₃ (23.3 g, 71.4 mmol) in anhydrous DMF (236 mL) was heated to 100° C. for 16 h. The reaction mixture was allowed to cool to rt and partitioned between EtOAc and H₂O and separated. The aqueous was re-extracted with EtOAc (3×) and the combined organic fractions were washed with H₂O (2×) and brine (2×), dried over Na₂SO₄, filtered and concentrated in vacuo resulting in the title compound as an orange oil. It was used in the next step without further purification. ¹H NMR (400 MHz, CDCl₃): δ=1.31 (s, 12H), 1.33 (s, 3H), 1.39 (s, 3H), 3.78 (dd, J=8.8, 5.9 Hz, 1H), 4.07 (dd, J=8.8, 6.2 Hz, 1H), 4.23-4.35 (m, 2H), 4.47 (quint, J=5.8 Hz, 1H), 7.78 (s, 1H), 7.81 (s, 1H).

### Example 9

### trans-4-(4-{3-[1-(2-Chloro-3-fluoro-6-methoxyphenyl)ethyl]-1H-pyrazolo[3,4-b]pyridin-5-yl}-1H-pyrazol-1-yl)cyclohexanol

A solution of 1-(trans-4-{[tert-butyl(dimethyl)silyl]oxy}cyclohexyl)-4-(4,4,5,5-tetramethyl-1,3,2-dioxaborolan-2-yl)-1H-pyrazole (42.3 mg, 0.104 mmol), 5-bromo-3-[1-(2-chloro-3-fluoro-6-methoxyphenyl)ethyl]-1H-pyrazolo[3,4-b]pyridine (20.0 mg, 0.0520 mmol), potassium carbonate (22.0 mg, 0.156 mmol), and 1,1′-bis(diphenylphosphino)ferrocenepalladium(II) dichloride, dichloromethane (4 mg, 0.005 mmol) in previously degassed 4:1 dioxane:water (1.8 mL) was evacuated and charged with N₂ gas (3×) and heated under microwave conditions [Biotage, 100° C., 30 min, high absorption]. The reaction mixture was partitioned between CHCl₃ and H₂O and separated. The aqueous was re-extracted with CHCl₃ (3×) and the combined organic fractions were dried over Na₂SO₄, filtered and concentrated in vacuo resulting in a crude yellow oil. The crude was further purified by chromatography on silica gel [Jones Flashmaster, eluting with 2% MeOH in CHCl₃] resulting in the TBDMS-protected title compound as a yellow oil. To it was added 4 M of HCl in 1,4-dioxane (1.0 mL), and the mixture was stirred at rt for 30 min. The reaction mixture was partitioned between CHCl₃ and H₂O and neutralized with sat. NaHCO₃ and separated. The aqueous was re-extracted with CHCl₃ (3×) and the combined organic fractions were dried over Na₂SO₄, filtered, and concentrated in vacuo resulting in the crude product that was further purified by chromatography on silica gel [Jones Flashmaster, eluting with 5% MeOH in CHCl₃] resulting in the title compound as an off-white solid. ¹H NMR (400 MHz, MeOD): δ=1.40-1.56 (m, 2H), 1.83-1.98 (m, 5H), 2.03-2.19 (m, 4H), 3.60 (s, 3H), 3.62-3.72 (m, 1H), 4.19 (tt, J=11.8, 3.7 Hz, 1H), 5.23 (q, J=7.0 Hz, 1H), 6.94 (dd, J=9.1, 4.3 Hz, 1H), 7.13-7.23 (m, 1H), 7.48 (d, J=1.5 Hz, 1H), 7.60 (s, 1H), 7.95 (s, 1H), 8.62 (d, J=2.0 Hz, 1H). MS (ES+): m/z=469.99/471.93 (76/24) [MH⁺]. HPLC: t_{R}=2.42 min (nonpolar_{—}5 min, ZQ3).

### 5-Bromo-3-[1-(2-chloro-3-fluoro-6-methoxyphenyl)ethyl]-1H-pyrazolo[3,4-b]pyridine

A solution of 1-(5-bromo-2-chloropyridin-3-yl)-2-(2-chloro-3-fluoro-6-methoxyphenyl)-propan-1-one (0.588 g, 1.44 mmol) in anhydrous i-PrOH (25.0 mL) was charged with hydrazine hydrate (0.464 mL, 9.53 mmol) and stirred at 80° C. for 3 h then allowed to stir at rt for an additional 48 h. The white precipitate was filtered through a fritted funnel and washed with EtOAc resulting in the title compound as a white solid. The filtrate was concentrated in vacuo and triturated with EtOAc/1-PrOH and filtered, resulting in an additional crop of the title compound as an off-white solid. ¹H NMR (400 MHz, DMSO-d₆): δ=1.80 (d, J=7.1 Hz, 3H), 3.32 (s, 3H), 5.10 (q, J=7.2 Hz, 1H), 7.05 (dd, J=4.3, 9.1 Hz, 1H), 7.35 (dd, J=9.0, 9.0 Hz, 1H), 7.59 (d, J=2.3 Hz, 1H), 8.50 (d, J=2.0 Hz, 1H). MS (ES+): m/z 383.85, 385.83, 387.81 (100/68/17) [MH⁺]. HPLC: t_{R}=3.1 min (nonpolar_{—}5 min, ZQ3).

### 1-(5-Bromo-2-chloropyridin-3-yl)-2-(2-chloro-3-fluoro-6-methoxyphenyl)propan-1-one

A solution of 1-(5-bromo-2-chloropyridin-3-yl)-2-(2-chloro-3-fluoro-6-methoxyphenyl)propan-1-ol (0.843 g, 2.06 mmol) in anhydrous DCM (23 mL) was charged with pyridinium chlorochromate (1.78 g, 8.24 mmol) at rt and stirred for 16 h. The reaction mixture was charged with an additional amount of pyridinium chlorochromate (1.00 g, 4.64 mmol) and stirred for an additional 24 h at rt and for 1 h at 40° C. The reaction mixture was concentrated in vacuo and diluted with ether and filtered through a pad of celite and the celite pad was washed with ether (4 volumes) and the filtrate was concentrated in vacuo resulting in a dark brown oil. This was purified by chromatography on silica gel [ISCO Combiflash, 40 g cartridge, 100% heptane→14% EtOAc in heptane] resulting in the title compound as clear colorless oil. ¹H NMR (400 MHz, CDCl₃): δ=1.51 (d, J=7.0 Hz, 3H), 3.72 (s, 3H), 4.88 (q, J=6.6 Hz, 1H), 6.59 (dd, J=3.9, 9.0 Hz, 1H), 6.98 (dd, J=8.8 Hz, 8.8 Hz, 1H), 7.79 (d, J=2.2 Hz, 1H), 8.37 (d, J=2.6 Hz, 1H). MS (ES+): m/z 405.81, 407.80, 409.77 (100/68/17) [MH⁺]. HPLC: t_{R}=3.43 min (nonpolar_{—}5 min, ZQ3).

### 1-(5-Bromo-2-chloropyridin-3-yl)-2-(2-chloro-3-fluoro-6-methoxyphenyl)propan-1-ol

A solution of 5-bromo-2-chloro-3-iodopyridine (0.750 g, 2.36 mmol) in anhydrous THF (5.5 mL) was cooled to −50° C. and dropwise charged with 2.0 M of isopropylmagnesium chloride in THF (1.41 mL, 2.83 mmol) over an 8 min period and the mixture was stirred at −50° C. for an additional 30 min. After 30 min., the mixture was charged with 2-(2-chloro-3-fluoro-6-methoxyphenyl)propanal (0.766 g, 3.53 mmol) and stirred at −40° C. for 1 h then allowed to warm to 0° C. and charged with brine (10 mL) and allowed to stir for 15 min. The reaction mixture was partitioned between EtOAc and H₂O and separated. The aqueous was re-extracted with EtOAc (3×) and the combined organic fractions were dried over Na₂SO₄, filtered and concentrated in vacuo resulting in 930 mg of a crude oil/solid mixture. The mixture was recrystallized from 20% EtOAc in hexanes resulting in 245 mg of a white solid (diastereomer A). The mother liquor was purified by chromatography on silica gel [Jones Flashmaster, 20 g cartridge, eluting with 12% EtOAc in hexanes] resulting in 311 mg of a white foam (mainly diastereomer B). These two diastereomers were combined for the subsequent oxidation step. Diastereomer A: ¹H NMR (400 MHz, CDCl₃): δ=1.34 (d, J=6.23 Hz, 3H), 3.62 (br. s., 1H), 3.89 (br. s., 3H), 5.43 (br. s., 1H), 6.70-6.80 (m, 1H), 6.94-7.03 (m, 1H), 8.11 (d, J=1.5 Hz, 1H), 8.30 (d, J=1.5 Hz, 1H). MS (ES+): m/z 407.73, 409.77, 411.74 [MH⁺]. HPLC: t_{R}=3.12 min (nonpolar_{—}5 min, ZQ3). Diastereomer B: ¹H NMR (400 MHz, CDCl₃): δ=1.41 (d, J=7.3 Hz, 3H), 3.93-3.97 (m, 3H), 3.97-4.05 (m, 1H), 5.44 (br. s., 1H), 5.55 (dd, J=4.6, 6.6 Hz, 1H), 6.83 (dd, J=4.3, 9.1 Hz, 1H), 7.03 (dd, J=8.1, 9.1 Hz, 1H), 7.76 (d, J=0.5 Hz, 1H), 8.33 (d, J=2.5 Hz, 1H). MS (ES+): m/z 407.73, 409.76, 411.74 (100/68/17) [MH⁺]. HPLC: t_{R}=3.35 min (nonpolar_{—}5 min, ZQ3).

### 2-(2-Chloro-3-fluoro-6-methoxyphenyl)propanal

Into a 250 mL single-necked round-bottom flask was charged the previously prepared crude 2-[2-chloro-3-fluoro-6-methoxyphenyl]propionitrile (5.3 g, 25.4 mmol) together with 160 mL of toluene. The mixture was cooled to 0-5° C. with stirring and DIBAL (25% w/w solution in hexanes; 16.2 g, 4.5 eq.) was added slowly over 5 min. The same temperature was maintained for about 2.5 h. The reaction mixture was poured into a 1 L separating funnel to which 250 mL of ether was added followed by 100 mL of water and 100 mL of 2N HCl. The organic layer was separated and the aqueous layer was extracted again with ether (150 mL). Both ether layers were combined, washed with brine (100 mL) then dried over the anhydrous sodium sulfate, filtered and concentrated on a rotary evaporator to provide the title compound as a clear oil. ¹H NMR (CDCl₃, 300 MHz): δ=1.39 (d, J=6.9 Hz, 3H), 3.75 (s, 3H), 3.94 (q, J=6.9 Hz, 1H), 6.78 (m, 1H), 7.13 (t, J=8.7 Hz, 1H), 9.6 (s, 1H).

### 2-[2-Chloro-3-fluoro-6-methoxyphenyl]propionitrile

Into a 500 mL two-necked round-bottom flask was charged the crude 2-chloro-3-(1-chloroethyl)-1-fluoro-4-methoxybenzene (9.10 g, 41 mmol) together with 250 mL of dry DMF. Sodium cyanide (12.05 g, 245 mmol, 6 eq.) was then added in one portion to flask and the temperature of the mixture was raised to 75° C. and maintained at this temperature with stirring overnight. The mixture was then poured into a 1 L separating funnel together with 250 mL of ether and 150 mL of 10% aq. sodium bicarbonate solution. The ether layer was separated and the aqueous layer was washed with water (3×250 mL) then with brine (2×75 mL). It was dried over sodium sulfate, filtered and concentrated on a rotary evaporator to give the title compound as an oil that was used in the next step without further purification. ¹H NMR (CDCl₃, 300 MHz): δ=1.61 (d, J=6.9 Hz, 3H), 3.97 (s, 3H), 4.59 (q, J=6.9 Hz, 1H), 6.79 (m, 1H), 7.13 (t, J=8.7 Hz, 1H).

### 2-Chloro-3-(1-chloroethyl)-1-fluoro-4-methoxybenzene

Into a 250 mL single-necked round-bottom flask were charged 1-(2-chloro-3-fluoro-6-methoxyphenyl)ethanol (6.5 g, 32 mmol) and 80 mL of dichloromethane. The clear solution was cooled to 0° C. and triethylamine (19.3 g, 192 mmol, 6 eq.) was added in one portion. After stirring for 10 min, methanesulfonyl chloride (16.6 g, 128 mmole, 4 eq.) was added drop-wise over a period of 15 min, and the reaction mixture was stirred overnight at 24° C. The reaction mixture was quenched with 60 mL of water and then extracted with ethyl acetate (2×100 mL). The combined organic layer was washed with water, dried over anhydrous sodium sulfate, filtered and then concentrated using a rotary evaporator to give the title compound as an oil that was used without further purification in the next step. ¹H NMR (CDCl₃, 300 MHz): δ=1.94 (d, J=3.3 Hz, 3H), 3.89 (s, 3H), 5.82 (brs, 1H), 6.78 (m, 1H), 7.06 (t, J=4.5 Hz, 1H).

### 1-(2-Chloro-3-fluoro-6-methoxyphenyl)ethanol

To a solution of 2-Chloro-3-fluoro-6-methoxybenzaldehyde (1.000 g, 5.303 mmol) in THF (20 mL, 200 mmol) at 0° C. was added 1.4 M of methylmagnesium bromide in THF (7.6 mL, 10.6 mmol), and the mixture was allowed to warm to rt for 3 h. The reaction was quenched with sat. NH₄Cl and the organic solvent was removed in vacuo. The residue was partitioned between DCM and water, and the organic layer was dried with magnesium sulfate, filtered, and concentrated in vacuo to afford the title compound as a pale yellow oil. ¹H NMR (CDCl₃, 400 MHz): δ=7.01 (dd, J=9.2, 8.8 Hz, 1H), 6.78 (dd, J=9.2, 4.0 Hz, 1H), 5.33 (brs, 1H), 3.90 (s, 3H), 3.81 (brs, 1H), 1.54 (d, J=7.2 Hz, 3H). MS (ES+): m/z=186.96/188.99 (100/45) [MH⁺-H₂O]. HPLC: t_{R}=3.05 min (polar_{—}5 min, ZQ3).

### 2-Chloro-3-fluoro-6-methoxybenzaldehyde

To a solution of 2-chloro-1-fluoro-4-methoxybenzene (28.5 g, 178 mmol) in t-butyl methyl ether (200 mL, dried over anhydrous MgSO₄) at −78° C. was added 2.5 M n-butyl lithium in hexanes (107 mL, 268 mmol). After 3 h, methyl formate (18.76 mL) was added dropwise while keeping the temperature below −60° C. The reaction mixture was quenched with sat. aq. ammonium chloride (250 mL) after 45 minutes and the organic layer was separated. The aq. layer was extracted with ethyl acetate (2×100 mL), and the combined organic layers were washed with water (200 mL) followed by brine, dried (Na₂SO₄) and concentrated to give a residue which on trituration with hexanes gave solids. The solids were filtered, taken again in hexanes and heated over a steam bath. The mixture was cooled, and the light yellow desired product filtered off and air-dried to give the title compound. ¹H NMR (400 MHz, CDCl₃): δ=10.48 (d, J=0.8 Hz, 1H), 7.31 (dd, J=9.4, 7.8 Hz, 1H), 6.88 (dd, J=7.8, 3.8 Hz, 1H), 3.92 (s, 3H).

### Alternative synthesis of 5-Bromo-3-[1-(2-chloro-3-fluoro-6-methoxyphenyl)ethyl]-1H-pyrazolo[3,4-b]pyridine

5-Bromo-3-[1-(2-chloro-3-fluoro-6-methoxyphenyl)ethyl]-1H-pyrazolo[3,4-b]pyridine was prepared following the procedure described above, replacing 1-(5-bromo-2-chloropyridin-3-yl)-2-(2-chloro-3-fluoro-6-methoxyphenyl)propan-1-one with 1-(5-bromo-2-fluoropyridin-3-yl)-2-(2-chloro-3-fluoro-6-methoxyphenyl)propan-1-one.

### 1-(5-Bromo-2-fluoropyridin-3-yl)-2-(2-chloro-3-fluoro-6-methoxyphenyl)propan-1-one

A solution of 1-(5-bromo-2-fluoropyridin-3-yl)-2-(2-chloro-3-fluoro-6-methoxyphenyl)propan-1-ol (0.237 g, 0.604 mmol) in anhydrous DCM (8.0 mL) was charged with pyridinium chlorochromate (0.520 g, 2.41 mmol) and heated to 40° C. for 8 h. The reaction mixture was concentrated in vacuo and diluted with diethyl ether and filtered through a pad of celite. The crude reaction mixture was washed with ether until no more product was observed and filtered through the pad of celite. The filtrate was concentrated in vacuo and purified by chromatography on silica gel [ISCO Combiflash, 12 g cartridge, 100% heptane-20% EtOAc in heptane] resulting in the title compound as an clear colorless oil. ¹H NMR (400 MHz, CDCl₃): δ=1.48 (d, J=6.8 Hz, 3H), 3.65 (s, 3H), 3.78-3.80 (m, 1H), 4.71 (qd, J=6.7, 1.9 Hz, 1H), 6.58 (dd, J=9.2, 4.2 Hz, 1H), 6.98 (dd, J=9.0, 8.5 Hz, 1H), 8.14 (dd, J=7.8, 2.5 Hz, 1H), 8.23 (dd, J=2.5, 1.0 Hz, 1H). MS (ES+): m/z=389.86/391.89/393.81 (100/68/17) [MH⁺]. HPLC: t_{R}=3.35 min (nonpolar_{—}5 min, ZQ3).

### 1-(5-Bromo-2-fluoropyridin-3-yl)-2-(2-chloro-3-fluoro-6-methoxyphenyl)propan-1-ol

A solution of 2.0 M of lithium diisopropylamide in THF (1.56 mL, 3.12 mmol) in anhydrous THF (6.3 mL) was cooled to −78° C. and dropwise charged with a solution of 5-bromo-2-fluoropyridine (0.500 g, 2.84 mmol) in anhydrous THF (6.3 mL) over a 5 min period and stirred at −78° C. for 30 min. The reaction mixture was dropwise charged with a solution of 2-(2-chloro-3-fluoro-6-methoxyphenyl)propanal (0.923 g, 4.26 mmol) in anhydrous THF (2.0 mL) at −78° C. and stirred for an additional 15 min at −78° C. and quenched with sat. ammonium chloride (3.0 mL) and allowed to reach rt. The reaction mixture was partitioned between EtOAc and H₂O and separated. The aqueous was back extracted with EtOAc (3×) and the combined organic fractions were dried over Na₂SO₄, filtered and concentrated in vacuo resulting in a crude yellow oil. The crude material was purified by chromatography on silica gel [ISCO Combiflash, 12 g cartridge, eluting with 100% heptane→20% EtOAc in heptane] resulting in 237 mg, 21% yield of the title compound as a clear colorless oil. MS (ES+): m/z=391.90/393.88/395.86 (100/68/17) [MH⁺]. HPLC: t_{R}=3.03, 3.24 min (nonpolar_{—}5 min, ZQ3).

### Example 10

### trans-4-(4-{3-[(1R)-1-(2-Chloro-3-fluoro-6-methoxyphenyl)ethyl]-1H-pyrazolo[3,4-b]pyridin-5-yl}-1H-pyrazol-1-yl)cyclohexanol

A solution of 1-(trans-4-{[tert-butyl(dimethyl)silyl]oxy}cyclohexyl)-4-(4,4,5,5-tetramethyl-1,3,2-dioxaborolan-2-yl)-1H-pyrazole (0.127 g, 0.312 mmol), 5-bromo-3-[(1R)-1-(2-chloro-3-fluoro-6-methoxyphenyl)ethyl]-1H-pyrazolo[3,4-b]pyridine (0.0800 g, 0.208 mmol), potassium carbonate (0.0860 g, 0.624 mmol), and 1,1′-bis(diphenylphosphino)ferrocenepalladium(II) dichloride dichloromethane (16.9 mg, 0.0210 mmol) in previously degassed 4:1 dioxane:water (7.2 mL) was evacuated and charged with N₂ gas (3×) and heated under microwave conditions [Biotage, 100° C., 30 min, high absorption]. The reaction mixture was charged with 4 M of HCl in 1,4-dioxane (4.0 mL) and heated in under microwave conditions using the Biotage [60° C., 15 min, high absorption]. The reaction mixture was partitioned between CHCl₃ and H₂O and neutralized with sat. NaHCO₃ and separated. The aqueous was re-extracted with CHCl₃ (3×) and the combined organic fractions were dried over Na₂SO₄, filtered, and concentrated in vacuo resulting in 54 mg of a crude product that was further purified by chromatography on silica gel [Jones Flashmaster, eluting with 2.5% MeOH in CHCl₃] resulting in the title compound as a tan solid. ¹H NMR (400 MHz, CD₃OD): δ=1.44-1.57 (m, 1H), 1.87-1.99 (m, 4H), 2.13 (t, J=13.5 Hz, 4H), 3.58-3.62 (m, 3H), 3.69 (tt, J=4.0, 10.9 Hz, 1H), 4.16-4.26 (m, 1H), 5.25 (q, J=7.1 Hz, 1H), 6.96 (dd, J=4.3, 9.1 Hz, 1H), 7.20 (t, J=8.8 Hz, 1H), 7.49 (d, J=2.0 Hz, 1H), 7.62 (s, 1H), 7.95-7.99 (m, 1H), 8.64 (d, J=2.0 Hz, 1H). MS (ES+): m/z 470.01, 471.99 (76/24) [MH⁺]. HPLC: t_{R}=2.34 min (nonpolar_{—}5 min, ZQ3).

### 5-Bromo-3-[(1R)-(2-chloro-3-fluoro-6-methoxyphenyl)ethyl]-1H-pyrazolo[3,4-b]pyridine and 5-Bromo-3-[(1S)-(2-chloro-3-fluoro-6-methoxyphenyl)ethyl]-1H-pyrazolo[3,4-b]pyridine

The racemic compound was separated by chiral SFC [CHIRAL PAK iB (21×250 mm/5μ), 30% MeOH: flow rate 30 mL/min, S enantiomer elutes first, R enantiomer elutes second]. (5-bromo-3-[(1S)-1-(2-chloro-3-fluoro-6-methoxyphenyl)ethyl]-1H-pyrazolo[3,4-b]pyridine): ¹H NMR (400 MHz, DMSO-d₆): δ=1.80 (d, J=7.0 Hz, 3H), 3.60 (s, 3H), 5.10 (q, J=7.0 Hz, 1H), 7.05 (dd, J=4.4, 9.2 Hz, 1H), 7.35 (dd, J=8.9, 8.9 Hz, 1H), 7.60 (d, J=2.3 Hz, 1H), 8.51 (d, J=2.0 Hz, 1H), 13.5 (br. s., 5H). MS (ES+): m/z 383.85, 385.83, 387.85 (100/68/17) [MH⁺]. HPLC: t_{R}=3.1 min (nonpolar_{—}5 min, ZQ3). (5-bromo-3-[(1R)-1-(2-chloro-3-fluoro-6-methoxyphenyl)ethyl]-1H-pyrazolo[3,4-b]pyridine): ¹H NMR (400 MHz, DMSO-d₆) δ=1.80 (d, J=7.0 Hz, 3H), 3.60 (s, 3H), 5.10 (q, J=7.0 Hz, 1H), 7.05 (dd, J=4.4, 9.2 Hz, 1H), 7.35 (dd, J=9.0, 9.0 Hz, 1H), 7.60 (d, J=2.0 Hz, 1H), 8.51 (d, J=2.0 Hz, 1H), 13.53 (br. s., 1H). MS (ES+): m/z 383.85, 385.84, 387.85 (100/68/17) [MH⁺]. HPLC: t_{R}=3.1 min (nonpolar_{—}5 min, ZQ3).

### Example 11

### trans-4-(4-{3-[(1S)-1-(2-Chloro-3-fluoro-6-methoxyphenyl)ethyl]-1H-pyrazolo[3,4-b]pyridin-5-yl}-1H-pyrazol-1-yl)cyclohexanol

A solution of 1-(trans-4-{[tert-butyl(dimethyl)silyl]oxy}cyclohexyl)-4-(4,4,5,5-tetramethyl-1,3,2-dioxaborolan-2-yl)-1H-pyrazole (0.127 g, 0.312 mmol), 5-bromo-3-[(1S)-1-(2-chloro-3-fluoro-6-methoxyphenyl)ethyl]-1H-pyrazolo[3,4-b]pyridine (0.0800 g, 0.208 mmol), potassium carbonate (0.086 g, 0.624 mmol), and 1,1′-bis(diphenylphosphino)ferrocenepalladium(II) dichloride, dichloromethane (17.0 mg, 0.0208 mmol) in previously degassed 4:1 Dioxane:water (7.2 mL) was evacuated and charged with N₂ gas (3×) and heated under microwave conditions [Biotage, 100° C., 30 min, high absorption]. The reaction vial was charged with 4 M of HCl in 1,4-dioxane (1.0 mL) and heated under microwave conditions [Biotage, 60° C., 15 min, high absorption]. The reaction mixture was partitioned between EtOAc and H₂O and separated. The aqueous was back extracted with EtOAc (3×) and the combined organic fractions were washed with sat. NaHCO₃ (1×), brine (1×), dried over Na₂SO₄, filtered and concentrated in vacuo resulting in a crude brown oil. The crude was purified by chromatography on silica gel [Jones Flashmaster, eluting with 2.5% MeOH in CHCl₃] resulting in the title compound as a tan solid. ¹H NMR (400 MHz, CD₃OD): δ=1.42-1.55 (m, 2H), 1.85-1.97 (m, 5H), 2.11 (t, J=13.5 Hz, 4H), 3.58 (s, 3H), 3.67 (tt, J=4.2, 10.8 Hz, 1H), 4.19 (tt, J=3.8, 11.8 Hz, 1H), 5.23 (q, J=7.0 Hz, 1H), 6.95 (dd, J=4.3, 9.1 Hz, 1H), 7.18 (dd, J=8.8, 8.8 Hz, 1H), 7.48 (d, J=2.0 Hz, 1H), 7.60 (s, 1H), 7.95 (s, 1H), 7.60 (s, 1H), 8.62 (d, J=2.0 Hz, 1H). MS (ES+): m/z 470.01, 471.99 (76/24) [MH⁺]. HPLC: t_{R}=2.41 min (nonpolar_{—}5 min, ZQ3).

### Example 12

### 3-[(1R)-1-(2-chloro-3-fluoro-6-methoxyphenyl)ethyl]-5-[1-(piperidin-4-yl)-1H-pyrazol-4-yl]-1H-pyrazolo[3,4-b]pyridine

A solution of 4-[4-(4,4,5,5-tetramethyl-1,3,2-dioxaborolan-2-yl)-pyrazol-1-yl]-piperidine-1-carboxylic acid tert-butyl ester (0.0363 g, 0.0962 mmol), 5-bromo-3-[(1R)-1-(2-chloro-3-fluoro-6-methoxyphenyl)ethyl]-1H-pyrazolo[3,4-b]pyridine (0.0247 g, 0.0642 mmol), potassium carbonate (0.0266 g, 0.192 mmol), and 1,1′-bis(diphenylphosphino)ferrocenepalladium(II) dichloride dichloromethane (5.25 mg, 0.00642 mmol) in previously degassed 4:1 dioxane:water (2.2 mL) was evacuated and charged with N₂ gas (3×) and heated under microwave conditions [Biotage, 100° C., 30 min, high absorption]. The reaction mixture was charged with 4 M of HCl in 1,4-dioxane (1.0 mL) and heated in under microwave conditions using the Biotage [60° C., 15 min, high absorption]. The reaction mixture was partitioned between CHCl₃ and H₂O and neutralized with sat. NaHCO₃ and separated. The aqueous was re-extracted with CHCl₃ (3×) and the combined organic fractions were dried over Na₂SO₄, filtered, and concentrated in vacuo resulting in crude product that was further purified by MDP chromatography. The resulting fractions were combined and partitioned between CHCl₃ and sat. NaHCO₃ and separated. The aqueous was back extracted with CHCl₃ (3×) and the combined organic fractions were dried over Na₂SO₄, filtered and concentrated in vacuo resulting in the title compound as an off-white solid. ¹H NMR (400 MHz, CD₃OD): δ=1.89-2.05 (m, 5H), 2.14 (d, J=9.9 Hz, 2H), 2.75-2.86 (m, 2H), 3.23 (d, J=12.9 Hz, 2H), 3.60 (s, 3H) 4.34 (tt, J=4.1, 11.5 Hz, 1H), 5.25 (q, J=7.1 Hz, 1H), 6.97 (dd, J=4.2, 9.2 Hz, 1H), 7.20 (dd, J=8.8, 8.8 Hz, 1H), 7.50 (d, J=2.0 Hz, 1H), 7.64 (s, 1H), 7.98 (s, 1H), 8.66 (d, J=2.0 Hz, 1H). MS (ES+): m/z 455.00, 456.98 (76/24) [MH⁺]. HPLC: t_{R}=2.30 min (nonpolar_{—}5 min, ZQ3).

### Example 13

### (2S)-3-(4-{3-[(1R)-1-(2-chloro-3-fluoro-6-methoxyphenyl)ethyl]-1H-pyrazolo[3,4-b]pyridin-5-yl}-1H-pyrazol-1-yl)propane-1,2-diol

A solution of 1-{[(4S)-2,2-dimethyl-1,3-dioxolan-4-yl]methyl}-4-(4,4,5,5-tetramethyl-1,3,2-dioxaborolan-2-yl)-1H-pyrazole (0.0297 g, 0.0964 mmol), 5-bromo-3-[(1R)-1-(2-chloro-3-fluoro-6-methoxyphenyl)ethyl]-1H-pyrazolo[3,4-b]pyridine (0.0247 g, 0.0642 mmol), potassium carbonate (0.0266 g, 0.192 mmol), and 1,1′-bis(diphenylphosphino)ferrocenepalladium(II) dichloride, dichloromethane (5.24 mg, 0.00642 mmol) in previously degassed 4:1 dioxane:water (2.2 mL) was evacuated and charged with N₂ gas (3×) and heated under microwave conditions [Biotage, 100° C., 30 min, high absorption]. The reaction mixture was charged with 4 M of HCl in 1,4-dioxane (1.2 mL) and heated in under microwave conditions using the Biotage [60° C., 15 min, high absorption]. The reaction mixture was partitioned between CHCl₃ and H₂O and neutralized with sat. NaHCO₃ and separated. The aqueous was re-extracted with CHCl₃ (3×) and the combined organic fractions were dried over Na₂SO₄, filtered, and concentrated in vacuo resulting in 51 mg of a crude product that was further purified by MDP. After MDP, the fractions were combined and partitioned between CHCl₃ and sat. NaHCO₃ and separated. The aqueous was back extracted with CHCl₃ (3×) and the combined organic fractions were washed with brine (1×), dried over Na₂SO₄, filtered and concentrated in vacuo resulting in the title compound as an off-white solid. ¹H NMR (400 MHz, DMSO-d₆): δ=1.85 (d, J=7.1 Hz, 3H), 3.33-3.41 (m, 2H), 3.63 (s, 3H), 3.79-3.86 (m, 1H), 3.99 (dd, J=7.8, 13.6 Hz, 1H), 4.23 (dd, J=3.9, 13.8 Hz, 1H), 4.74 (t, J=5.6 Hz, 1H), 4.98 (d, J=5.3 Hz, 1H), 5.15 (q, J=7.1 Hz, 1H), 7.06 (dd, J=4.4, 9.2 Hz, 1H), 7.34 (dd, J=9.0, 9.0 Hz, 1H), 7.53 (d, J=1.8 Hz, 1H), 7.72 (d, J=0.76 Hz, 1H), 8.04 (s, 1H), 8.69 (d, J=2.0 Hz, 1H), 13.21 (s, 1H). MS (ES+): m/z 445.93, 447.91 (76/24) [MH⁺]. HPLC: t_{R}=2.10 min (nonpolar_{—}5 min, ZQ3).

### Example 14

### 3-[(1R)-1-(2-chloro-3-fluoro-6-methoxyphenyl)ethyl]-5-(1-methyl-1H-pyrazol-4-yl)-1H-pyrazolo[3,4-b]pyridine

A solution of 1-methyl-4-(44,5,5-tetramethyl-1,3,2-dioxaborolan-2-yl)-1H-pyrazole (0.0200 g, 0.0961 mmol, 5-bromo-3-[(1R)-1-(2-chloro-3-fluoro-6-methoxyphenyl)ethyl]-1H-pyrazolo[3,4-b]pyridine (0.0247 g, 0.0642 mmol), potassium carbonate (0.0266 g, 0.192 mmol), and 1,1′-bis(diphenylphosphino)ferrocenepalladium(II) dichloride, dichloromethane (5.24 mg, 0.00642 mmol) in previously degassed 4:1 dioxane:water (2.2 mL) was evacuated and charged with N₂ gas (3×) and heated under microwave conditions [Biotage, 100° C., 30 min, high absorption]. The reaction mixture was irradiated under microwave conditions for an additional 30 min. The reaction mixture was partitioned between CHCl₃ and H₂O and separated and the aqueous was back extracted with CHCl₃ (3×) and the combined organic fractions were dried over Na₂SO₄, filtered and concentrated in vacuo resulting in a crude brown oil. The crude material was purified by chromatography on silica gel [ISCO Combiflash, 4 g cartridge, eluting with 100% DCM→4% MeOH in DCM] resulting in the title compound as an orange solid. ¹H NMR (400 MHz, DMSO-d₆): δ=1.85 (d, J=7.3 Hz, 3H), 3.63 (s, 3H), 3.86 (s, 3H), 5.15 (q, J=7.0 Hz, 1H), 7.05 (dd, J=4.4, 9.2 Hz, 1H), 7.34 (dd, J=9.0, 9.0 Hz, 1H), 7.52 (d, J=1.8 Hz, 1H), 7.71 (d, J=0.76 Hz, 1H), 8.06 (s, 1H), 8.68 (d, J=2.0 Hz, 1H), 13.22 (s, 1H). MS (ES+): m/z 385.97, 387.95 (76/24) [MH⁺]. HPLC: t_{R}=2.53 min (nonpolar_{—}5 min, ZQ3).

### Example 15

### 5-[1-(2,6-Dichloro-3-fluorophenyl)ethyl]-3-(1-methyl-1H-pyrazol-4-yl)-7H-pyrrolo[2,3-c]pyridazine

To a solution of 5-[(2,6-dichloro-3-fluorophenyl)(methoxy)methyl]-3-(1-methyl-1H-pyrazol-4-yl)-7H-pyrrolo[2,3-c]pyridazine (100 mg, 0.246 mmol) in anhydrous THF (5 mL) was added BF₃.OEt₂ (0.216 mL 0.861 mmol) at −50° C. The resulting solution was stirred for 10 min at the same temperature and then 2M solution of ZnMe₂ in toluene (0.86 mL, 0.86 mmol) was added. The solution was allowed to warm to room temperature over 1 hour and was stirred at 60° C. overnight. It was then cooled down to −78° C. and quenched by saturated aqueous NH₄Cl solution (100 mL). The organic layer was separated, dried over Na₂SO₄ and concentrated to provide a crude residue which was first purified by silica gel column chromatography eluting with 10% methanol in methylene chloride. The yellow solid thus obtained was dissolved in a mixture of MeOH and DMF, syringe filtered, and purified by MDP, under acidic conditions (formic acid). Fractions containing product were combined and concentrated in vacuo, affording the title compound as formate salt as a yellow solid. ¹H NMR (400 MHz, CDCl₃): δ=1.95 (d, J=7.3 Hz, 3H), 4.03 (s, 3H), 5.26-5.34 (m, 1H), 7.09 (t, J=8.3 Hz, 1H), 7.36 (br s, 1H), 7.55 (s, 1H), 7.84 (s, 1H), 8.35 (s, 1H), 8.82 (s, 1H), 12.49 (br s, 1H). MS (ES+): m/z=389.99/391.97 (100/96) [MH⁺]. HPLC: t_{R}=2.93 min (ZQ3, polar_{—}5 min).

### 5-[(2,6-Dichloro-3-fluorophenyl)(methoxy)methyl]-3-(1-methyl-1H-pyrazol-4-yl)-7H-pyrrolo[2,3-c]pyridazine

To a solution of 3-(1-methyl-1H-pyrazol-4-yl)-7H-pyrrolo[2,3-c]pyridazine (110 mg, 5.07 mmol) and 2,6-dichloro-3-fluorobenzaldehyde (129 mg, 0.670 mmol, 1.2 eq) in methanol (10 mL) in a sealed tube was added potassium hydroxide (56 mg, 1 mmol, 1.8 eq) and stirred at 110° C. overnight. The reaction mixture was poured into water; the solid that precipitated was filtered and washed with isopropyl ether. It was purified by column chromatography on silica gel eluting with 5 to 10% methanol in methylene chloride to yield the title compound as white solid. ¹HNMR (300 MHz, CDCl₃): δ=3.49 (s, 3H), 4.00 (s, 3H), 6.49 (s, 1H), 7.16 (dd, J=7.8, 8.1 Hz, 1H), 7.39 (dd, J=7.8, 8.1 Hz, 1H), 7.50 (s, 1H), 7.80 (s, 1H), 7.97 (s, 1H), 8.05 (s, 1H).

### 3-(1-Methyl-1H-pyrazol-4-yl)-7H-pyrrolo[2,3-c]pyridazine

Through a well stirred suspension of 3-chloro-7H-pyrrolo[2,3-c]pyridazine (200 mg, 1.3 mmol), 1-methyl-4-[4-(4,4,5,5-tetramethyl[1,3,2]dioxaborolan-2-yl)-1H-pyrazole (324 mg, 1.55 mmol, 1.2 eq) and cesium carbonate (848 mg, 2.6 mmol, 2 eq) in 20% aqueous dioxane (40 mL) was bubbled N₂ gas for 15 min. at room temp. To the resulting solution was then added Pd(PPh₃)₄ (72 mg, 0.06 mmol) and heated at 100° C. for 13 h. The reaction mixture was cooled to RT and solvent was removed under reduced pressure. The residue was partitioned between DCM and water (50 mL each), and the aqueous layer was extracted with methylene chloride (3×100 mL). The combined organic layers were washed with water (20 mL) followed by brine (10 mL), dried over sodium sulfate, filtered, and concentrated under reduced pressure to yield a brown solid, which was purified by column chromatography on silica gel using 5 to 10% MeOH in DCM as eluent to yield the title compound as brown solid. ¹H NMR (300 MHz, CDCl₃): δ=4.01 (s, 3H), 6.52 (d, J=3.3 Hz, 1H), 7.71 (d, J=3.3 Hz, 1H), 7.88 (s, 1H), 8.03 (s, 1H), 8.09 (s, 1H).

### 3-Chloro-7H-pyrrolo[2,3-c]pyridazine

A mixture of 6-chloro-4-[(trimethylsilyl)ethynyl]pyridazin-3-amine (400 mg, 1.76 mmol) and CuI (67 mg, 0.36 mmol, 0.2 eq) in NMP (5 mL) was stirred for 10 min in a sealed tube and then heated at 190° C. in a microwave reactor for 30 seconds. The dark reaction mixture was then cooled to room temperature and solvent was removed under vacuum to yield a dark brown residue. It was purified by column chromatography by eluting with 2% methanol in methylene chloride to yield the title compound as brown solid. ¹H NMR (300 MHz, CDCl₃): δ=6.54 (d, J=3.3 Hz, 1H), 7.80 (s, 1H), 7.87 (d, J=3.3 Hz, 1H).

### 6-Chloro-4-[(trimethylsilyl)ethynyl]pyridazin-3-amine

Through a well stirred suspension of 4-bromo-6-chloropyridazin-3-amine (12 g, 42.8 mmol), ethynyltrimethylsilane (4.6 g, 47.1 mmol, 1.1 eq), CuI (900 mg, 4.2 mmol, 0.1 eq), and triethylamine (7.8 mL, 54.6 mmol, 2.5 eq) in toluene (200 mL) was bubbled nitrogen for 15 min. PdCl₂(PPh₃)₂ (3.0 g, 4.28 mmol, 0.1 eq.) was added to the reaction mixture and stirred at room temperature for 16 h. Toluene was removed under reduced pressure to yield a brown solid to which water (50 mL) and ethyl acetate (50 mL) were added. The organic layer was separated and the aqueous layer was extracted with ethyl acetate (2×20 mL). The combined organic extracts were washed with water (20 mL), followed by brine (20 mL), dried over sodium sulfate, filtered, and concentrated under reduced pressure to yield a dark brown residue, which was purified by column chromatography by eluting with 2% to 10% EtOAc in hexane to afford the title compound as brown solid. ¹HNMR (300 MHz, CDCl₃): δ=0.29 (s, 9H), 5.25 (s, 2H), 7.25 (s, 1H). MS (ES+): m/z=495/497 [MH⁺].

### 4-Bromo-6-chloropyridazin-3-amine

To a well stirred suspension of 6-chloropyridazin-3-amine (10.0 g, 77.2 mmol) and sodium bicarbonate (12.9 g, 154 mmol) in methanol (150 mL) was added bromine (12.4 g, 77.2 mmol) dropwise, and the resulting mixture was stirred at room temperature for 18 h. The reaction mixture was filtered and the solid residue was washed with methanol (3×15 mL). The filtrate was concentrated in vacuo to yield semi-solid material to which water (50 mL) and ethyl acetate (50 mL) were added. The organic layer was separated and the aqueous layer was extracted with ethyl acetate (2×50 mL). The combined organic layers were washed with 10% aq. sodium thiosulfate (2×50 mL), followed by brine (20 mL), dried over sodium sulfate, filtered, and concentrated under vacuum. The residue was purified by column chromatography on silica gel eluting with 50% ethyl acetate in hexane to yield the title compound. ¹H NMR (300 MHz, CDCl₃): δ=6.38 (s, 2H), 7.54 (s, 1H).

### Example 16

### 5-[1-(2,6-Dichloro-3-fluorophenyl)ethyl]-3-[1-(piperidin-4-yl)-1H-pyrazol-4-yl]-7H-pyrrolo[2,3-c]pyridazine

To a solution of tert-butyl 4-(4-{5-[(2,6-dichloro-3-fluorophenyl)(methoxy)methyl]-7H-pyrrolo[2,3-c]pyridazin-3-yl}-1H-pyrazol-1-yl)piperidine-1-carboxylate (120 mg, 0.209 mmol) in anhydrous THF (5 mL) was added BF₃.OEt₂ (0.184 mL 1.46 mmol, 7 eq.) at 50° C. The resulting solution was stirred for 10 min at the same temperature and then 2M solution of ZnMe₂ in toluene (0.73 mL, 1.46 mmol, 7 eq.) was added. The resulting mixture was allowed to warm up to room temperature in 1 hour. The solution was then stirred at 60° C. overnight. It was then cooled down to 78° C. and quenched by saturated aqueous NH₄Cl solution (100 mL). The organic layer was separated, dried over Na₂SO₄ and concentrated to provide a crude residue which was first purified by silica gel column chromatography eluting with 10% methanol. The yellow film thus obtained was dissolved in MeOH and purified by MDP, under acidic conditions (TFA). The fractions containing product were combined and concentrated in vacuo, affording the title compound as trifluoroacetate as a yellow solid. ¹H NMR (400 MHz, CDCl₃): δ=1.98 (d, J=7.3 Hz, 3H), 2.47 (br d, J=12.6 Hz, 2H), 2.82-2.98 (m, 2H), 3.26-3.39 (m, 2H), 3.75 (br d, J=12.6 Hz, 2H), 4.64-4.77 (m, 1H), 5.34 (q, J=7.2 Hz, 1H), 7.13 (dd, J=8.8, 7.8 Hz, 1H), 7.32 (br s, 1H), 7.66 (s, 1H), 7.77 (s, 1H), 8.27 (s, 1H), 8.87 (br s, 1H), 9.11 (br s, 1H), 10.31 (br s, 1H), 13.70 (br s, 1H). MS (ES+): m/z=458.95/460.96 (100/69) [MH⁺]. HPLC: t_{R}=4.3 min (MDPZQ, polar_{—}10 min). [LCMS was recorded using the analytical mode of the MDPS because TFA was required to get a sharp peak.]

### tert-Butyl 4-(4-{5-[(2,6-dichloro-3-fluorophenyl)(methoxy)methyl]-7H-pyrrolo[2,3-c]pyridazin-3-yl}-1H-pyrazol-1-yl)piperidine-1-carboxylate

To a solution of tert-butyl 4-[4-(7H-pyrrolo[2,3-c]pyridazin-3-yl)-1H-pyrazol-1-yl]piperidine-1-carboxylate (110 mg, 5.07 mmol) and 2,6-dichloro-3-fluorobenzaldehyde (69 mg, 0.36 mmol, 1.2 eq) in methanol (10 mL) in a sealed tube was added potassium hydroxide (30 mg, 0.54 mmol, 1.8 eq) and stirred at 110° C. for 16 h. The reaction mixture was poured into water; the solid that precipitated out was filtered off and washed with isopropyl ether. It was purified by column chromatography on silica gel, eluting with 2 to 5% methanol in methylene chloride to yield the title compound as white solid. ¹H NMR (300 MHz, CDCl₃): δ=1.42 (s, 9H), 1.82-2.35 (m, 4H), 2.77-3.09 (m, 4H), 3.49 (s, 3H), 3.51 (s, 1H), 4.32 (m_{c}, 1H), 6.49 (s, 1H), 7.20 (m, 1H), 7.35-7.42 (m, 1H), 7.49 (s, 1H), 7.81 (s, 1H), 7.96 (s, 1H), 8.14 (s, 1H).

### tert-Butyl 4-[4-(7H-pyrrolo[2,3-c]pyridazin-3-yl)-1H-pyrazol-1-yl]piperidine-1-carboxylate

To a well stirred suspension of 3-chloro-7H-pyrrolo[2,3-c]pyridazine (136 mg, 0.89 mmol), 4-[4-(4,4,5,5-tetramethyl-1,3,2-dioxaborolan-2-yl)pyrazol-1-yl]-piperidine-1-carboxylic acid tert-butyl ester (367 mg, 0.97 mmol, 1.1 eq), and cesium carbonate (526 mg, 1.62 mmol, 1.8 eq) in 20% aqueous dioxane (20 mL) was bubbled nitrogen for 15 min at room temperature. To the resulting mixture was added Pd(PPh₃)₄ (51 mg, 0.045 mmol), then the mixture was heated at 100° C. for 16 h. The reaction mixture was cooled to RT and concentrated under reduced pressure. The residue was partitioned between water and DCM (30 mL each), and the aqueous layer was extracted with more methylene chloride (2×15 mL). The combined organic layers were washed with water (20 mL) followed by brine (10 mL), dried over sodium sulfate, filtered, and concentrated under reduced pressure. The residue was purified by column chromatography on silica gel, eluting with 5 to 10% methanol in methylene chloride as eluent to yield the title compounds as off-white solid. ¹H NMR (300 MHz, CDCl₃): δ=1.45 (s, 9H), 1.81-2.35 (m, 4H), 2.78-3.05 (m, 4H), 4.12 (m_{c}, 1H), 6.62 (d, J=3.3 Hz, 1H), 7.62 (d, J=3.3 Hz, 1H), 7.93 (s, 1H), 8.08 (s, 1H), 8.20 (s, 1H).

### Biological Properties

In some aspects, as discussed above, compounds of the invention are inhibitors of kinases, including at least one of the MET, RON, ALK, IR, and IGF-1R kinases.

In some aspects, as discussed above, compounds of the invention are inhibitors of kinases, including at least one of MET, RON, ALK, IR, IGF-1R, Trk, Tie-2, Flt3, FGFR3, Abl, Jak2, Alk, c-Src, PAK1, PAK2, AXL, and TAK1 kinases. In some further aspects, compounds of the invention are inhibitors of kinases, including one or more of Blk, c-Raf, PRK2, Lck, Mek1, PDK-1, GSK313, EGFR, p70S6K, BMX, SGK, CaMKII, and Tie-2 kinases.

In some aspects, as discussed above, compounds of the invention are selective inhibitors of at least one or MET, RON, and ALK. In some aspects, as discussed above, compounds of the invention are selective inhibitors of at least one or MET, RON, IR, IGF-1R, and ALK. In some embodiments, the compound is a selective inhibitor MET and/or RON over other kinase targets, such as KDR.

Thus, in some aspects, a compound or salt thereof as described herein, exhibits inhibition of MET in a cellular assay with an IC₅₀ of about 50 nM or less, 100 nM or less, or 200 nM or less.

In some aspects, a compound or salt thereof as described herein, exhibits inhibition of RON in a cellular assay with an IC₅₀ of about 200 nM or less or 500 nM or less.

In some aspects, a compound or salt thereof as described herein, exhibits inhibition of MET in a cellular assay with an IC₅₀ as described above and inhibition of RON in a cellular assay with an IC₅₀ as described above.

In some aspects, the compound or salt thereof is about 10-fold or more selective for MET over KDR. In some aspects, compounds of the invention are useful as selective inhibitors of one or more of MET, RON, and ALK with selectivity over AKB and/or KDR of 2, 4, 8, 16, or 32-fold, or greater.

In some aspects, compounds of the invention inhibit epithelial to mesenchymal transition.

The effect of inhibitors on the proliferation of MKN45 cells was determined using the following protocol. MKN45 cells were plated in Corning 3917 96-well white tissue culture treated plates in growth medium (RPMI, 10% FCS) at a density of 5000 cells/well in a total volume of 135 μL and incubated at 37° C., 5% CO₂, 95% humidity overnight. The following day, one-tenth volume of a 10× concentration of compounds was added to the wells in an 8-point dilution series. The dilution series was composed of an initial 1:5 dilution of a 10 mM stock of compound in DMSO, followed by serial 1:4 dilutions in DMSO, then a 1:20 dilution in growth medium prior to the 1:10 dilution into the cell plate. Final DMSO concentration on the cells was 0.1%, there were control wells treated with both 0.1% DMSO and no DMSO. The typical dilution range is 10 μM to 0.6 nM. Once the compound was added to the cells, plates were incubated for 3 days at 37° C., 5% CO₂ at 95% humidity. On the third day, after allowing all cells and reagents to come to room temperature, 25 μL of CellTiter-Glo reagent (Promega # G7573) was added to the wells. Plates were shaken on a platform for 10 minutes prior to reading luminescence for 0.1 seconds. The signal of the control wells was taken as 100% growth and growth inhibition was expressed as percent of control. IC₅₀ values were determined from the percent of control data using a standard four-parameter model.

The IC₅₀ values of exemplary compounds of the present invention determined in a cell proliferation assay using the MKN45 cell line according to the procedures described herein in at least duplicate experiments are abbreviated as follows and are shown in Table 1: A, IC₅₀≦0.1 μM; B, 0.1 μM<IC₅₀≦0.5 μM; C, 0.5 μM<IC₅₀≦2 μM; D, IC₅₀>2 μM; ND, not determined. The Example # of Table 1 corresponds to the compound example number as illustrated in the Experimental section above.

MKN45 is a human gastric carcinoma cell line that shows a high level of amplification of MET and constitutive activation of MET. Treatment of this cell line with a selective MET inhibitor led to induction of apoptosis and inhibition of proliferation, whereas non-MET-amplified cell lines were not affected. Smolen et al., Proc. Natl. Acad. Sci. USA, 103(7):2316-2321 (2006). This cell line is thus “driven” by MET, and antiproliferative effects correlate very well with the inhibition of MET phosphorylation so that the cell proliferation IC₅₀ values can be used as surrogate for the MET cell mechanistic IC₅₀ values. Under the assay conditions described herein, the IC₅₀ values correlate nearly 1:1.

TABLE 1
IC₅₀ values of examples in MKN45 cell proliferation assay

| | Example | | | | | | | |
|---|---|---|---|---|---|---|---|---|
| | 1 | 2 | 3 | 4 | 5 | 6 | 7 | 8 |
| Prolif. IC₅₀ | ND | B | C | C | A | B | B | B |
| | Example | | | | | | | |
| | 9 | 10 | 11 | 12 | 13 | 14 | 15 | 16 |
| Prolif. IC₅₀ | A | A | D | A | B | B | D | D |

The cellular activity of the compounds of the present invention against MET may be determined by the following procedure. MKN45 cells were plated in Falcon 3072 96-well plates in growth media (RPMI, 10% FBS, 1% L-glutamine) at a density of 5000 cells/well and incubated at 37° C., 5% CO₂ overnight. The following day, one-tenth volume of a 10× concentration of compounds was added to the wells in a 6-point dilution series. The dilutions series was composed of an initial 1:5 dilution in DMSO, followed by a 1:10 dilution in growth media, for a final DMSO concentration on cells of 0.5%. Control wells were treated with 0.5% DMSO. The typical range of dilution was 10 μM to 3 nM. Once compound was added to the cells, plates were incubated for four hours at 37° C., 5% CO₂. Plates were then washed in PBS, and lysed in triton-based lysis buffer. Lysates were transferred to a precoated capture plate made by Biosource (Cat # KHO0281). The phosphorylated MET levels were measured by incubating with a rabbit polyclonal antibody against phosphorylated MET ([pYpYpY1230/1234/1235]) followed by an anti-rabbit antibody conjugated to HRP. Signal was measured on a Wallac Victor plate reader at 450 nm. The DMSO signal of the control wells was defined as 100% and the percent of inhibition of phosphorylated MET was expressed as percent of control. IC₅₀ values were determined from the percent of control data using a standard four-parameter model.

The IC₅₀ values of exemplary compounds of the present invention determined in a MET cell mechanistic assay using the MKN45 cell line according to the procedures described herein in at least duplicate experiments are abbreviated as follows and are shown in Table 2: A, IC₅₀≦0.1 μM; B, 0.1 μM<IC₅₀≦0.5 μM; C, 0.5 μM<IC₅₀≦2 μM; D, IC₅₀>2 μM; ND, not determined. The Example # of Table 2 corresponds to the compound example number as illustrated in the Experimental section above.

TABLE 2
IC₅₀ values of examples in MET cell mechanistic assay (MKN45)

| | Example | | | | | | | |
|---|---|---|---|---|---|---|---|---|
| | 1 | 2 | 3 | 4 | 5 | 6 | 7 | 8 |
| MET mech IC₅₀ | B | B | B | ND | ND | ND | ND | ND |

Example

| | 9 | 10 | 11 | 12 | 13 | 14 | 15 | 16 |
|---|---|---|---|---|---|---|---|---|
| MET mech | ND | ND | ND | ND | ND | ND | ND | D |
| IC₅₀ | | | | | | | | |

The cellular activity of the compounds of the present invention against RON may be determined by the following procedure. HeLa cells were plated in Falcon 3072 96-well plates in growth media (DMEM, 10% FBS, 1% L-glutamine) at a density of 10000 cells/well and incubated at 37° C., 5% CO₂ overnight. The following day, cells were transfected with 0.2 μg sfRON-pcDNA plasmid DNA with 0.5 μL Lipofectamine-2000 per well in the presence of 50 μL OPTI-MEM, incubated at 37° C., 5% CO₂ overnight. Costar 3915 96-well assay plates were coated with rabbit Anti-RON antibody at 2.0 μg/mL, sealed, and incubated overnight at 4° C. On the third day, coated plates were washed with PBS and blocked with 3% BSA. For the sfRON transfected cells, one-tenth volume of a 10× concentration of compounds was added to the wells in a 6-point dilution series. The dilution series was composed of an initial 1:5 dilution of a 10 mM DMSO stock solution of compound in DMSO, followed by a 1:10 dilution in growth media, for a final DMSO concentration on cells of 0.5%. Control wells were treated with 0.5% DMSO. The typical range of dilution was 10 μM to 3 nM. Once compound was added to the cells, plates were incubated for four hours at 37° C., 5% CO₂. Plates were then washed in PBS, and lysed in triton-based lysis buffer. Lysates were transferred to the blocked capture plates. The phosphorylated RON levels were measured by incubating with a Goat polyclonal antibody against phosphorylated RON ([pYpY1238/1239]) followed by an anti-Goat antibody conjugated to HRP. Signal was measured on a Wallac Victor plate reader with luminance. The DMSO signal of the control wells was defined as 100% and the percent of inhibition of phosphorylated RON was expressed as percent of control. IC₅₀ values were determined from the percent of control data using a standard four-parameter model.

The IC₅₀ values of exemplary compounds of the present invention determined in a sfRON cell mechanistic assay using the HeLa cell line according to the procedures described herein in at least duplicate experiments are abbreviated as follows and are shown in Table 3: A, IC₅₀≦0.1 μM; B, 0.1 μM<IC₅₀≦0.5 μM; C, 0.5 μM<IC₅₀≦1 μM; D, IC₅₀>1 μM; ND, not determined. The Example # of Table 3 corresponds to the compound example number as illustrated in the Examples section.

TABLE 3
IC₅₀ values of examples in sfRON cell mechanistic assay (HeLa)

| | Example | | | | | | | |
|---|---|---|---|---|---|---|---|---|
| | 1 | 2 | 3 | 4 | 5 | 6 | 7 | 8 |
| sfRON mech IC₅₀ | ND | ND | ND | D | C | C | B | C |

| | Example | | | | | | | |
|---|---|---|---|---|---|---|---|---|
| | 9 | 10 | 11 | 12 | 13 | 14 | 15 | 16 |
| sfRON mech IC₅₀ | ND | B | ND | ND | ND | ND | ND | ND |

### Compositions

The invention includes pharmaceutical compositions comprising a compound or pharmaceutically acceptable salt thereof of the invention, which is formulated for a desired mode of administration with or without one or more pharmaceutically acceptable and useful carriers. The compounds can also be included in pharmaceutical compositions in combination with one or more other therapeutically active compounds.

The pharmaceutical compositions of the present invention comprise a compound of the invention (or a pharmaceutically acceptable salt thereof) as an active ingredient, optional pharmaceutically acceptable carrier(s) and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

Compounds of the invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula I, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

A formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Compounds of the invention can be provided for formulation at high purity, for example at least about 90%, 95%, or 98% pure by weight.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

### Uses

Compounds of the invention are useful to inhibit the activity of tyrosine kinase enzymes, including in animals, including humans, and can be useful in the treatment and/or prevention of various diseases and conditions such as hyperproliferative disorders such as cancer. In particular, compounds disclosed herein are inhibitors of kinases, including at least one of the MET, RON, ALK, IR, and IGF-1R kinases.

In some further aspects, compounds of the invention can be used as inhibitors of kinases, including one or more of MET, RON, ALK, IR, IGF-1R, Trk, Tie-2, Flt3, FGFR3, Abl, Jak2, Alk, c-Src, PAK1, PAK2, AXL, and TAK1 kinases. In some further aspects, compounds of the invention can be used as inhibitors of kinases, including one or more of Blk, c-Raf, PRK2, Lck, Mek1, PDK-1, GSK313, EGFR, p70S6K, BMX, SGK, CaMKII, and Tie-2 kinases.

In some aspects, compounds of the invention are useful to selectively inhibit one or more of MET and/or RON and/or ALK. In some aspects, the compound or salt thereof is a dual RON and MET inhibitor. In some embodiments, the compound is useful as a selective inhibitor of MET and/or RON and/or ALK over other kinase targets, such as KDR and/or Aurora kinase B (AKB). In some aspects, compounds of the invention are useful as selective inhibitors of one or more of MET, RON, and ALK with selectivity over AKB and/or KDR of 2, 4, 8, 16, or 32-fold, or greater.

In some aspects, the invention includes a method of treating cancer, tumors, and tumor metastases, comprising administering to a mammal in need thereof a therapeutically effective amount of a compound or salt of the invention.

In some aspects, compounds of the invention are in particular useful in treating proliferative disease, particularly cancers, including cancers mediated by MET and/or RON and/or ALK, alone or in combination with other agents.

In some aspects, compounds of the invention inhibit epithelial to mesenchymal transition (EMT).

In view of the above, compounds of Formula I of the present invention can be useful in the treatment of a variety of cancers, including, but not limited to, solid tumor, sarcoma, fibrosarcoma, osteoma, melanoma, retinoblastoma, rhabdomyosarcoma, glioblastoma, neuroblastoma, teratocarcinoma, hematopoietic malignancy, and malignant ascites. More specifically, the cancers include, but not limited to, lung cancer, bladder cancer, pancreatic cancer, kidney cancer, gastric cancer, breast cancer, colon cancer, prostate cancer (including bone metastases), hepatocellular carcinoma, ovarian cancer, esophageal squamous cell carcinoma, melanoma, an anaplastic large cell lymphoma, an inflammatory myofibroblastic tumor, and a glioblastoma.

In some aspects, the above methods are used to treat one or more of bladder, colorectal, nonsmall cell lung, breast, or pancreatic cancer. In some aspects, the above methods are used to treat one or more of ovarian, gastric, head and neck, prostate, hepatocellular, renal, glioma, glioma, or sarcoma cancer.

In some aspects, there is provided a method of treating a cancer selected from bladder, colorectal, non-small cell lung, breast, or pancreatic, ovarian, gastric, head and neck, prostate, hepatocellular, renal, glioma, or sarcoma cancer comprising administering to a mammal in need thereof a therapeutically effective amount of a compound or salt of the invention.

In some aspects, the invention includes a method, including the above methods, wherein the compound is used to inhibit EMT.

In some aspects, the invention includes a method of treating cancer comprising administering to a mammal in need thereof a therapeutically effective amount of a compound or salt of the invention, wherein at least one additional active anti-cancer agent is used as part of the method. In some aspects, the additional agent(s) is an EGFR inhibitor and/or an IGF-1R inhibitor.

Generally, dosage levels on the order of from about 0.01 mg/kg to about 150 mg/kg of body weight per day are useful in the treatment of the above-indicated conditions, or alternatively about 0.5 mg to about 7 g per patient per day. For example, inflammation, cancer, psoriasis, allergy/asthma, disease and conditions of the immune system, disease and conditions of the central nervous system (CNS), may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

In some aspects, the invention includes a method of treating cancer comprising administering to a mammal in need thereof a therapeutically effective amount of a compound or salt of the invention, wherein at least one additional active anti-cancer agent is used as part of the method.

### General Definitions and Abbreviations

Except where otherwise indicated, the following general conventions and definitions apply. Unless otherwise indicated herein, language and terms are to be given their broadest reasonable interpretation as understood by the skilled artisan. Any examples given are nonlimiting.

Any section headings or subheadings herein are for the reader's convenience and/or formal compliance and are non-limiting.

A recitation of a compound herein is open to and embraces any material or composition containing the recited compound (e.g., a composition containing a racemic mixture, tautomers, epimers, stereoisomers, impure mixtures, etc.). In that a salt, solvate, or hydrate, polymorph, or other complex of a compound includes the compound itself, a recitation of a compound embraces materials containing such forms. Isotopically labeled compounds are also encompassed except where specifically excluded. For example, hydrogen is not limited to hydrogen containing zero neutrons.

The term “active agent” of the invention means a compound of the invention in any salt, polymorph, crystal, solvate, or hydrated form.

The term “pharmaceutically acceptable salt(s)” is known in the art and includes salts of acidic or basic groups which can be present in the compounds and prepared or resulting from pharmaceutically acceptable bases or acids.

The term “substituted” and substitutions contained in formulas herein refer to the replacement of one or more hydrogen radicals in a given structure with a specified radical, or, if not specified, to the replacement with any chemically feasible radical. When more than one position in a given structure can be substituted with more than one substituent selected from specified groups, the substituents can be either the same or different at every position (independently selected) unless otherwise indicated. In some cases, two positions in a given structure can be substituted with one shared substituent. It is understood that chemically impossible or highly unstable configurations are not desired or intended, as the skilled artisan would appreciate.

In descriptions and claims where subject matter (e.g., substitution at a given molecular position) is recited as being selected from a group of possibilities, the recitation is specifically intended to include any subset of the recited group. In the case of multiple variable positions or substituents, any combination of group or variable subsets is also contemplated.

Unless indicated otherwise, a substituent, diradical or other group referred to herein can be bonded through any suitable position to a referenced subject molecule. For example, the term “indolyl” includes 1-indolyl, 2-indolyl, 3-indolyl, etc.

The convention for describing the carbon content of certain moieties is “(C_{a-b})” or “Cₐ-C_{b}” meaning that the moiety can contain any number of from “a” to “b” carbon atoms. C₀alkyl means a single covalent chemical bond when it is a connecting moiety, and a hydrogen when it is a terminal moiety. Similarly, “x-y” can indicate a moiety containing from x to y atoms, e.g., ₅₋₆heterocycloalkyl means a heterocycloalkyl having either five or six ring members. “C_{x-y}” may be used to define number of carbons in a group. For example, “C₀₋₁₂alkyl” means alkyl having 0-12 carbons, wherein C₀alkyl means a single covalent chemical bond when a linking group and means hydrogen when a terminal group.

The term “absent,” as used herein to describe a structural variable (e.g., “—R— is absent”) means that diradical R has no atoms, and merely represents a bond between other adjoining atoms, unless otherwise indicated.

Unless otherwise indicated (such as by a connecting “−”), the connections of compound name moieties are at the rightmost recited moiety. That is, the substituent name starts with a terminal moiety, continues with any bridging moieties, and ends with the connecting moiety. For example, “heteroarylthioC₁₋₄alkyl is a heteroaryl group connected through a thio sulfur to a C₁₋₄ alkyl, which alkyl connects to the chemical species bearing the substituent.

The term “aliphatic” means any hydrocarbon moiety, and can contain linear, branched, and cyclic parts, and can be saturated or unsaturated. The term includes, e.g., alkyl, alkenyl, alkynyl, cycloalkyl, carbocyclic, and others.

The term “alkyl” means any saturated hydrocarbon group that is straight-chain or branched. Examples of alkyl groups include methyl, ethyl, propyl, 2-propyl, n-butyl, iso-butyl, tert-butyl, pentyl, and the like.

The term “alkenyl” means any ethylenically unsaturated straight-chain or branched hydrocarbon group. Representative examples include, but are not limited to, ethenyl, 1-propenyl, 2-propenyl, 1-, 2-, or 3-butenyl, and the like.

The term “alkynyl” means any acetylenically unsaturated straight-chain or branched hydrocarbon group. Representative examples include, but are not limited to, ethynyl, 1-propynyl, 2-propynyl, 1-, 2-, or 3-butynyl, and the like.

The term “alkoxy” means —O-alkyl, —O-alkenyl, or —O-alkynyl. “Haloalkoxy” means an —O-(haloalkyl) group. Representative examples include, but are not limited to, trifluoromethoxy, tribromomethoxy, and the like.

“Haloalkyl” means an alkyl, preferably lower alkyl, that is substituted with one or more same or different halo atoms.

“Hydroxyalkyl” means an alkyl, preferably lower alkyl, that is substituted with one, two, or three hydroxy groups; e.g., hydroxymethyl, 1 or 2-hydroxyethyl, 1,2-, 1,3-, or 2,3-dihydroxypropyl, and the like.

The term “alkanoyl” means —C(O)-alkyl, —C(O)-alkenyl, or —C(O)-alkynyl.

“Alkylthio” means an —S-(alkyl) or an —S-(unsubstituted cycloalkyl) group. Representative examples include, but are not limited to, methylthio, ethylthio, propylthio, butylthio, cyclopropylthio, cyclobutylthio, cyclopentylthio, cyclohexylthio, and the like.

The term “cyclic” means any ring system with or without heteroatoms (N, O, or S(O)₀₋₂), and which can be saturated or unsaturated. Ring systems can be bridged and can include fused rings. The size of ring systems may be described using terminology such as “_{x-y}cyclic,” which means a cyclic ring system that can have from x to y ring atoms. For example, the term “₉₋₁₀carbocyclic” means a 5, 6 or 6,6 fused bicyclic carbocyclic ring system which can be satd., unsatd. or aromatic. It also means a phenyl fused to one 5 or 6 membered satd. or unsatd. carbocyclic group. Nonlimiting examples of such groups include naphthyl, 1,2,3,4 tetrahydronaphthyl, indenyl, indanyl, and the like.

The term “carbocyclic” means a cyclic ring moiety containing only carbon atoms in the ring(s) without regard to aromaticity. A 3-10 membered carbocyclic means chemically feasible monocyclic and fused bicyclic carbocyclics having from 3 to 10 ring atoms. Similarly, a 4-6 membered carbocyclic means monocyclic carbocyclic ring moieties having 4 to 6 ring carbons, and a 9-10 membered carbocyclic means fused bicyclic carbocyclic ring moieties having 9 to 10 ring carbons.

The term “cycloalkyl” means a non-aromatic 3-12 carbon mono-cyclic, bicyclic, or polycyclic aliphatic ring moiety. Cycloalkyl can be bicycloalkyl, polycycloalkyl, bridged, or spiroalkyl. One or more of the rings may contain one or more double bonds but none of the rings has a completely conjugated pi-electron system. Examples, without limitation, of cycloalkyl groups are cyclopropane, cyclobutane, cyclopentane, cyclopentene, cyclohexane, cyclohexadiene, adamantane, cycloheptane, cycloheptatriene, and the like.

The term “unsaturated carbocyclic” means any cycloalkyl containing at least one double or triple bond. The term “cycloalkenyl” means a cycloalkyl having at least one double bond in the ring moiety.

The terms “bicycloalkyl” and “polycycloalkyl” mean a structure consisting of two or more cycloalkyl moieties that have two or more atoms in common. If the cycloalkyl moieties have exactly two atoms in common they are said to be “fused”. Examples include, but are not limited to, bicyclo[3.1.0]hexyl, perhydronaphthyl, and the like. If the cycloalkyl moieties have more than two atoms in common they are said to be “bridged”. Examples include, but are not limited to, bicyclo[2.2.1]heptyl (“norbornyl”), bicyclo[2.2.2]octyl, and the like.

The term “spiroalkyl” means a structure consisting of two cycloalkyl moieties that have exactly one atom in common. Examples include, but are not limited to, spiro[4.5]decyl, spiro[2.3]hexyl, and the like.

The term “aromatic” means a planar ring moieties containing 4n+2 pi electrons, wherein n is an integer.

The term “aryl” means an aromatic moieties containing only carbon atoms in its ring system. Non-limiting examples include phenyl, naphthyl, and anthracenyl. The terms “aryl-alkyl” or “arylalkyl” or “aralkyl” refer to any alkyl that forms a bridging portion with a terminal aryl.

“Aralkyl” means alkyl, preferably lower alkyl, that is substituted with an aryl group as defined above; e.g., —CH₂ phenyl, —(CH₂)₂-phenyl, —(CH₂)₃ phenyl, CH₃CH(CH₃)CH₂-phenyl, and the like and derivatives thereof.

The term “heterocyclic” means a cyclic ring moiety containing at least one heteroatom (N, O, or S(O)₀₋₂), including heteroaryl, heterocycloalkyl, including unsaturated heterocyclic rings.

The term “heterocycloalkyl” means a non-aromatic monocyclic, bicyclic, or polycyclic heterocyclic ring moiety of 3 to 12 ring atoms containing at least one ring having one or more heteroatoms. The rings may also have one or more double bonds. However, the rings do not have a completely conjugated pi-electron system. Examples of heterocycloalkyl rings include azetidine, oxetane, tetrahydrofuran, tetrahydropyran, oxepane, oxocane, thietane, thiazolidine, oxazolidine, oxazetidine, pyrazolidine, isoxazolidine, isothiazolidine, tetrahydrothiophene, tetrahydrothiopyran, thiepane, thiocane, azetidine, pyrrolidine, piperidine, N-methylpiperidine, azepane, 1,4-diazapane, azocane, [1,3]dioxane, oxazolidine, piperazine, homopiperazine, morpholine, thiomorpholine, 1,2,3,6-tetrahydropyridine and the like. Other examples of heterocycloalkyl rings include the oxidized forms of the sulfur-containing rings. Thus, tetrahydrothiophene-1-oxide, tetrahydrothiophene-1,1-dioxide, thiomorpholine-1-oxide, thiomorpholine-1,1-dioxide, tetrahydrothiopyran-1-oxide, tetrahydrothiopyran-1,1-dioxide, thiazolidine-1-oxide, and thiazolidine-1,1-dioxide are also considered to be heterocycloalkyl rings. The term “heterocycloalkyl” also includes fused ring systems and can include a carbocyclic ring that is partially or fully unsaturated, such as a benzene ring, to form benzofused heterocycloalkyl rings. For example, 3,4-dihydro-1,4-benzodioxine, tetrahydroquinoline, tetrahydroisoquinoline and the like. The term “heterocycloalkyl” also includes heterobicycloalkyl, heteropolycycloalkyl, or heterospiroalkyl, which are bicycloalkyl, polycycloalkyl, or spiroalkyl, in which one or more carbon atom(s) are replaced by one or more heteroatoms selected from O, N, and S. For example, 2-oxa-spiro[3.3]heptane, 2,7-diaza-spiro[4.5]decane, 6-oxa-2-thia-spiro[3.4]octane, octahydropyrrolo[1,2-a]pyrazine, 7-aza-bicyclo[2.2.1]heptane, 2-oxa-bicyclo[2.2.2]octane, and the like, are such heterocycloalkyls.

Examples of saturated heterocyclic groups include, but are not limited to oxiranyl, thiaranyl, aziridinyl, oxetanyl, thiatanyl, azetidinyl, tetrahydrofuranyl, tetrahydrothiophenyl, pyrrolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, oxepanyl, thiepanyl, azepanyl, 1,4-dioxepanyl, 1,4-oxathiepanyl, 1,4-oxaazepanyl, 1,4-dithiepanyl, 1,4-thieazepanyl, 1,4-diazepanyl

Non-aryl heterocyclic groups include satd. and unsatd. systems and can include groups having only 4 atoms in their ring system. The heterocyclic groups include benzo-fused ring systems and ring systems substituted with one or more oxo moieties. Recitation of ring sulfur is understood to include the sulfide, sulfoxide or sulfone where feasible. The heterocyclic groups also include partially unsatd. or fully satd. 4-10 membered ring systems, e.g., single rings of 4 to 8 atoms in size and bicyclic ring systems, including aromatic 6-membered aryl or heteroaryl rings fused to a non-aromatic ring. Also included are 4-6 membered ring systems (“4-6 membered heterocyclic”), which include 5-6 membered heteroaryls, and include groups such as azetidinyl and piperidinyl. Heterocyclics can be heteroatom-attached where such is possible. For instance, a group derived from pyrrole can be pyrrol-1-yl (N-attached) or pyrrol-3-yl (C-attached). Other heterocyclics include imidazo[4,5-b]pyridin-3-yl and benzoimidazol-1-yl.

Examples of heterocyclic groups include pyrrolidinyl, tetrahydrofuranyl, tetrahydrothienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, and the like.

The term “unsaturated heterocyclic” means a heterocycloalkyl containing at least one unsaturated bond. The term “heterobicycloalkyl” means a bicycloalkyl structure in which at least one carbon atom is replaced with a heteroatom. The term “heterospiroalkyl” means a spiroalkyl structure in which at least one carbon atom is replaced with a heteroatom.

Examples of partially unsaturated heteroalicyclic groups include, but are not limited to: 3,4-dihydro-2H-pyranyl, 5,6-dihydro-2H-pyranyl, 2H-pyranyl, 1,2,3,4-tetrahydropyridinyl, and 1,2,5,6-tetrahydropyridinyl.

The terms “heteroaryl” or “hetaryl” mean a monocyclic, bicyclic, or polycyclic aromatic heterocyclic ring moiety containing 5-12 atoms. Examples of such heteroaryl rings include, but are not limited to, furyl, thienyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridyl, pyridazinyl, pyrimidinyl, pyrazinyl, and triazinyl. The terms “heteroaryl” also include heteroaryl rings with fused carbocyclic ring systems that are partially or fully unsaturated, such as a benzene ring, to form a benzofused heteroaryl. For example, benzimidazole, benzoxazole, benzothiazole, benzofuran, quinoline, isoquinoline, quinoxaline, and the like. Furthermore, the terms “heteroaryl” include fused 5-6, 5-5, 6-6 ring systems, optionally possessing one nitrogen atom at a ring junction. Examples of such hetaryl rings include, but are not limited to, pyrrolopyrimidinyl, imidazo[1,2-a]pyridinyl, imidazo[2,1-b]thiazolyl, imidazo[4,5-b]pyridine, pyrrolo[2,1-f][1,2,4]triazinyl, and the like. Heteroaryl groups may be attached to other groups through their carbon atoms or the heteroatom(s), if applicable. For example, pyrrole may be connected at the nitrogen atom or at any of the carbon atoms.

Heteroaryls include, e.g., 5 and 6 membered monocyclics such as pyrazinyl and pyridinyl, and 9 and 10 membered fused bicyclic ring moieties, such as quinolinyl. Other examples of heteroaryl include quinolin-4-yl, 7-methoxy-quinolin-4-yl, pyridin-4-yl, pyridin-3-yl, and pyridin-2-yl. Other examples of heteroaryl include pyridinyl, imidazolyl, pyrimidinyl, pyrazolyl, triazolyl, pyrazinyl, tetrazolyl, furanyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like. Examples of 5-6 membered heteroaryls include, thiophenyl, isoxazolyl, 1,2,3-triazolyl, 1,2,3-oxadiazolyl, 1,2,3-thiadiazolyl, 1,2,4-triazolyl, 1,3,4-oxadiazolyl, 1,3,4-thiadiazolyl, 1,2,5-oxadiazolyl, 1,2,5-thiadiazolyl, pyridyl, pyridazinyl, pyrimidinyl, pyrazinyl, 1,2,4 oxadiazolyl, 1,2,5-triazinyl, 1,3,5-triazinyl, and the like.

“Heteroaralkyl” group means alkyl, preferably lower alkyl, that is substituted with a heteroaryl group; e.g., —CH₂ pyridinyl, —(CH₂)₂pyrimidinyl, —(CH₂)₃ imidazolyl, and the like, and derivatives thereof.

A pharmaceutically acceptable heteroaryl is one that is sufficiently stable to be attached to a compound of the invention, formulated into a pharmaceutical composition and subsequently administered to a patient in need thereof.

Examples of monocyclic heteroaryl groups include, but are not limited to: pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl.

Examples of fused ring heteroaryl groups include, but are not limited to: benzoduranyl, benzothiophenyl, indolyl, benzimidazolyl, indazolyl, benzotriazolyl, pyrrolo[2,3-b]pyridinyl, pyrrolo[2,3-c]pyridinyl, pyrrolo[3,2-c]pyridinyl, pyrrolo[3,2-b]pyridinyl, imidazo[4,5-b]pyridinyl, imidazo[4,5-c]pyridinyl, pyrazolo[4,3-d]pyridinyl, pyrazolo[4,3-c]pyridinyl, pyrazolo[3,4-c]pyridinyl, pyrazolo[3,4-b]pyridinyl, isoindolyl, indazolyl, purinyl, indolinyl, imidazo[1,2-a]pyridinyl, imidazo[1,5-a]pyridinyl, pyrazolo[1,5-a]pyridinyl, pyrrolo[1,2-b]pyridazinyl, imidazo[1,2-c]pyrimidinyl, quinolinyl, isoquinolinyl, cinnolinyl, azaquinazoline, quinoxalinyl, phthalazinyl, 1,6-naphthyridinyl, 1,7naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrimido[2,3-b]pyrazinyl, pyrimido[4,5-d]pyrimidinyl.

“Arylthio” means an —S-aryl or an —S-heteroaryl group, as defined herein. Representative examples include, but are not limited to, phenylthio, pyridinylthio, furanylthio, thienylthio, pyrimidinylthio, and the like and derivatives thereof.

The term “9-10 membered heterocyclic” means a fused 5, 6 or 6,6 bicyclic heterocyclic ring moiety, which can be satd., unsatd. or aromatic. The term “9-10 membered fused bicyclic heterocyclic” also means a phenyl fused to one 5 or 6 membered heterocyclic group. Examples include benzofuranyl, benzothiophenyl, indolyl, benzoxazolyl, 3H-imidazo[4,5-c]pyridin-yl, dihydrophthazinyl, 1H-imidazo[4,5-c]pyridin-1-yl, imidazo[4,5-b]pyridyl, 1,3 benzo[1,3]dioxolyl, 2H-chromanyl, isochromanyl, 5-oxo-2,3 dihydro-5H-[1,3]thiazolo[3,2-a]pyrimidyl, 1,3-benzothiazolyl, 1,4,5,6 tetrahydropyridazyl, 1,2,3,4,7,8 hexahydropteridinyl, 2-thioxo-2,3,6,9-tetrahydro-1H-purin-8-yl, 3,7-dihydro-1H-purin-8-yl, 3,4-dihydropyrimidin-1-yl, 2,3-dihydro-1,4-benzodioxinyl, benzo[1,3]dioxolyl, 2H-chromenyl, chromanyl, 3,4-dihydrophthalazinyl, 2,3-dihydro-1H-indolyl, 1,3-dihydro-2H-isoindol-2-yl, 2,4,7-trioxo-1,2,3,4,7,8-hexahydropteridin-yl, thieno[3,2-d]pyrimidinyl, 4-oxo-4,7-dihydro-3H-pyrrolo[2,3-d]pyrimidin-yl, 1,3-dimethyl-6-oxo-2-thioxo-2,3,6,9-tetrahydro-1H-purinyl, 1,2-dihydroisoquinolinyl, 2-oxo-1,3-benzoxazolyl, 2,3-dihydro-5H-1,3-thiazolo-[3,2-a]pyrimidinyl, 5,6,7,8-tetrahydro-quinazolinyl, 4-oxochromanyl, 1,3-benzothiazolyl, benzimidazolyl, benzotriazolyl, purinyl, furylpyridyl, thiophenylpyrimidyl, thiophenylpyridyl, pyrrolylpiridyl, oxazolylpyridyl, thiazolylpiridyl, 3,4-dihydropyrimidin-1-yl imidazolylpyridyl, quinoliyl, isoquinolinyl, quinazolinyl, quinoxalinyl, naphthyridinyl, pyrazolyl[3,4]pyridine, 1,2-dihydroisoquinolinyl, cinnolinyl, 2,3-dihydro-benzo[1,4]dioxin-4-yl, 4,5,6,7-tetrahydro-benzo[b]-thiophenyl-2-yl, 1,8-naphthyridinyl, 1,5-napthyridinyl, 1,6-naphthyridinyl, 1,7-napthyridinyl, 3,4-dihydro-2H-1,4-benzothiazine, 4,8-dihydroxy-quinolinyl, 1-oxo-1,2-dihydro-isoquinolinyl, 4-phenyl-[1,2,3]thiadiazolyl, and the like.

“Aryloxy” means an —O-aryl or an —O-heteroaryl group, as defined herein. Representative examples include, but are not limited to, phenoxy, pyridinyloxy, furanyloxy, thienyloxy, pyrimidinyloxy, pyrazinyloxy, and the like, and derivatives thereof.

One in the art understands that an “oxo” requires a second bond from the atom to which the oxo is attached. Accordingly, it is understood that oxo cannot be subststituted onto an aryl or heteroaryl ring.

The term “halo” means fluoro, chloro, bromo, or iodo.

“Acyl” means a —C(O)R group, where R can be selected from the nonlimiting group of hydrogen or optionally substituted lower alkyl, trihalomethyl, unsubstituted cycloalkyl, aryl. “Thioacyl” or “thiocarbonyl” means a —C(S)R″ group, with R as defined above.

The term “protecting group” means a suitable chemical group that can be attached to a functional group and removed at a later stage to reveal the intact functional group. Examples of suitable protecting groups for various functional groups are described in T. W. Greene and P. G. M. Wuts, Protective Groups in Organic Synthesis, 2d Ed., John Wiley and Sons (1991 and later editions); L. Fieser and M. Fieser, Fieser and Fieser's Reagents for Organic Synthesis, John Wiley and Sons (1994); and L. Paquette, ed. Encyclopedia of Reagents for Organic Synthesis, John Wiley and Sons (1995). The term “hydroxy protecting group”, as used herein, unless otherwise indicated, includes Ac, CBZ, and various hydroxy protecting groups familiar to those skilled in the art including the groups referred to in Greene.

As used herein, the term “pharmaceutically acceptable salt” means those salts which retain the biological effectiveness and properties of the parent compound and do not present insurmountable safety or toxicity issues.

The term “pharmaceutical composition” means an active compound in any form suitable for effective administration to a subject, e.g., a mixture of the compound and at least one pharmaceutically acceptable carrier.

As used herein, a “physiologically/pharmaceutically acceptable carrier” means a carrier or diluent that does not cause significant irritation to an organism and does not abrogate the biological activity and properties of the administered compound.

A “pharmaceutically acceptable excipient” means an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

The terms “treat,” “treatment,” and “treating” means reversing, alleviating, inhibiting the progress of, or partially or completely preventing the disorder or condition to which such term applies, or one or more symptoms of such disorder or condition. “Preventing” means treating before an infection occurs.

“Therapeutically effective amount” means that amount of the compound being administered which will relieve to some extent one or more of the symptoms of the disorder being treated, or result in inhibition of the progress or at least partial reversal of the condition.

The following abbreviations are used:

min. minute(s)

h hour(s)

d day(s)

RT or rt room temperature

t_{R} retention time

L liter

mL milliliter

mmol millimole

μmol micromole

equiv. or eq. equivalents

NMR nuclear magnetic resonance

MDP(S) mass-directed HPLC purification (system)

LC/MS liquid chromatography mass spectrometry

HPLC high performance liquid chromatography

TLC thin layer chromatography

CDCl₃ deuterated chloroform

CD₃OD or MeOD deuterated methanol

DMSO-d₆ deuterated dimethylsulfoxide

LDA lithium diisopropylamide

DCM dichloromethane

THF tetrahydrofuran

EtOAc ethyl acetate

MeCN acetonitrile

DMSO dimethylsulfoxide

Boc tert-butyloxycarbonyl

DME 1,2-dimethoxyethane

DMF N,N-dimethylformamide

DIPEA diisopropylethylamine

PS-DIEA polymer-supported diisopropylethylamine

PS-PPh₃-Pd polymer-supported Pd(PPh₃)₄

EDC 1-(3-dimethylaminopropyl)-3-ethylcarbodiimide

HOBt 1-hydroxybenzotriazole

DMAP 4-dimethylaminopyridine

TBTU O-(benzotriazol-1-yl)-N,N,N′,N′-tetramethyluronium tetrafluoroborate

TEMPO 2,2,6,6-tetramethylpiperidine-1-oxyl

TFA trifluoroacetic acid

1. A compound of Formula I: 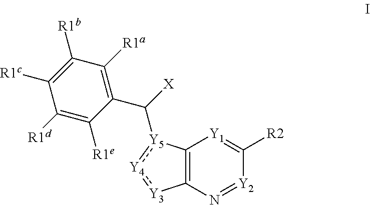
or a pharmaceutically acceptable salt thereof, wherein:
X is selected from H, C₁₋₃aliphatic or —OC₁₋₃aliphatic, either of which is optionally substituted with one or more halogen;
Y₁ and Y₂ are independently N or CH, except not more than one of Y₁ and Y₂ is N;
Y₃ is NH or CH; and when Y₃ is NH, then at least one of Y₁, Y₂, and Y₄ is N and Y₅ is C;
Y₄ is N or CH;
Y₅ is N or C, except not more than one of Y₄ and Y₅ is N;
R^{1a}, R^{1b}, R^{1c}, R^{1d}, R^{1e} are each independently optional substituents selected from aliphatic, cyclic, —O-aliphatic, —O-cyclic, sulfide, sulfone, sulfoxide, amino, amido, carboxyl, acyl, ureido, —S-cyclic, any of which is optionally substituted, halogen, or nitrile;
R2 is H or an optional substituent.

2. The compound or salt of claim 1, wherein:
R^{a1}, R^{1b}, R^{1c}, R^{1d}, R^{1e} are each independently selected from H, halo, —CN, C₁₋₆ alkyl, —CF₃, —OCF₃, —OCHF₂, —OC₀₋₆alkyl, —S(O)ₘC₁₋₆alkyl, —SO₂N(C₀₋₆alkyl)(C₀₋₆alkyl), —N(C₀₋₆alkyl)(C₀₋₆alkyl), —N(C₀₋₆alkyl)C(═O)C₀₋₆alkyl, —N(C₀₋₆alkyl)C(═O)OC₀₋₆alkyl, —N(C₀₋₆alkyl)C(═O)N(C₀₋₆alkyl)(C₀₋₆alkyl), —C(═O)C₀₋₆alkyl, —C(═O)OC₀₋₆alkyl, —C(═O)N(C₀₋₆alkyl)(C₀₋₆alkyl), —O-heterocyclyl, —N(C₀₋₆alkyl)-heterocyclyl, —N(C₀₋₆alkyl)-heteroaryl, heterocyclyl, heteroaryl, 5-heteroaryl, or —O-heteroaryl; wherein the heterocyclyl is optionally substituted with oxo, C₁₋₆alkyl, C(═O)OC₁₋₆alkyl, C(═O)C₀₋₆alkyl, C(═O)N(C₀₋₆alkyl)(C₀₋₆alkyl), SO₂N(C₀₋₆alkyl)(C₀₋₆alkyl), or SO₂C₁₋₆alkyl; wherein the alkyl is optionally substituted with —OH, —OC₁₋₆alkyl, N(C₀₋₆alkyl)(C₀₋₆alkyl), C(═O)N(C₀₋₆alkyl)(C₀₋₆alkyl), C(═O)OC₀₋₆alkyl, C(═O)C₀₋₆alkyl, heterocyclyl, or heteroaryl;
R² is selected from H, halo, —CN, —CF₃, —NO₂, C₀₋₆alkyl, C₂₋₆alkenyl, C₂₋₆alkynyl, C₃₋₆cycloalkylC₀₋₆alkyl, C₃₋₆heterocycloalkylC₀₋₆alkyl, arylC₀₋₆alkyl, or heteroarylC₀₋₆alkyl, any of which is optionally substituted with one or more independent G¹ substituents;
or R² is selected from: 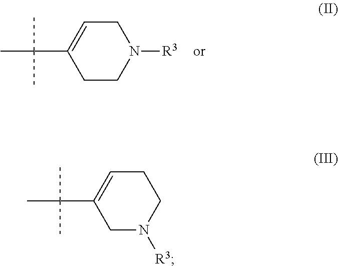
R³ is selected from H, C₁₋₁₂alkyl, R⁴O—C₂₋₁₂alkyl-, R⁴R⁵N—C₂₋₁₂alkyl-, R⁴S(O)ₘ—C₂₋₁₂alkyl, C₃₋₁₂cycloalkylC₀₋₁₂alkyl, C₃₋₁₂cycloalkenylC₁₋₁₂alkyl, heterocycloalkylC₀₋₁₂alkyl, arylC₀₋₁₂alkyl, heteroarylC₀₋₁₂alkyl, C₁₋₁₂alkylC₃₋₁₂cycloalkyl, C₃₋₁₂cycloalkylC₃₋₁₂cycloalkyl, C₃₋₁₂cycloalkenylC₃₋₁₂cycloalkyl, heterocycloalkylC₃₋₁₂cycloalkyl, arylC₃₋₁₂cycloalkyl, heteroarylC₃₋₁₂cycloalkyl, C₁₋₁₂alkyl-heterocycloalkyl, C₃₋₁₂cycloalkyl-heterocycloalkyl, C₃₋₁₂cycloalkenyl-heterocycloalkyl, heterocycloalkyl-heterocycloalkyl, aryl-heterocycloalkyl, heteroaryl-heterocycloalkyl, —C(O)R^{a}, R⁴O—C₀₋₁₂alkylC(O)—, R⁴R⁵N—C₀₋₁₂alkylC(O)—, R⁴S(O)ₘC₀₋₁₂alkylC(O)—, —CO₂R⁴, —C(O)NR⁴R⁵, —S(O)ₘR⁴, —SO₂NR⁴R⁵ or —C(S)OR⁴, any of which is optionally substituted with one or more independent G² substituents;
G¹ and G² are each independently selected from halo, —CN, —CF₃, —OCF₃, —NO₂, oxo, R⁶, C₁₋₁₂alkyl, C₂₋₁₂alkenyl, C₂₋₁₂alkynyl, C₃₋₁₂cycloalkylC₀₋₁₂alkyl, heterocycloalkylC₀₋₁₂alkyl, arylC₀₋₁₂alkyl, heteroarylC₀₋₁₂alkyl, —OR⁶, —S(O)ₘR⁶, —NR⁶R⁷, —SO₂NR⁶R⁷, —C(O)R^{b}, —C(O)NR⁶R⁷, —C(O)—C(O)NR⁶R⁷, —C(O)OR⁶, —C(O)—C(O)OR⁶, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, —NR¹⁰S(O)₂NR⁶R⁷, or —NR¹⁰S(O)NR⁶R⁷, any of which is optionally substituted with one or more independent Q¹ substituents;
Q¹ is selected from halo, —CN, —NO₂, oxo, —CF₃, —OCF₃, C₁₋₁₂alkyl, arylC₀₋₁₂alkyl, heteroarylC₀₋₁₂alkyl, C₃₋₁₂cycloalkylC₀₋₁₂alkyl, heterocycloalkylC₀₋₁₂alkyl, arylC₃₋₁₂cycloalkyl, heteroarylC₃₋₁₂cycloalkyl, heterocycloalkylC₃₋₁₂cycloalkyl, C₃₋₁₂cycloalkylC₃₋₁₂cycloalkyl, C₁₋₁₂alkyl-heterocycloalkyl, heterocycloalkyl-heterocycloalkyl, aryl-heterocycloalkyl, heteroaryl-heterocycloalkyl, —C(O)—C(O)NR¹¹R¹², —C(O)—C(O)OR¹¹, —OC(O)R^{c}, —NR¹¹C(O)R^{c}, —NR¹¹S(O)₂R¹², —(CR¹³R¹⁴)ₙC(O)R^{c}, —(CR¹³R¹⁴)ₙC(O)OR¹¹, —(CR¹³R¹⁴)ₙC(O)NR¹¹R¹², —(CR¹³R¹⁴)ₙS(O)₂NR¹¹R¹², —(CR¹³R¹⁴)ₙNR¹¹R¹², —(CR¹³R¹⁴)ₙOR¹¹, —(CR¹³R¹⁴)ₙS(O)ₘR¹¹, —NR¹⁵C(O)NR¹¹R¹², —NR¹⁵S(O)₂NR¹¹R¹² or —NR¹⁵S(O)NR¹¹R¹², any of which is optionally substituted with one or more independent Q² substituents;
Q² is selected from halo, —CN, —OH, —NH₂, —NO₂, oxo, —CF₃, —OCF₃, —CO₂H, —S(O)ₘH, C₁₋₁₂alkyl, arylC₀₋₁₂alkyl, heteroarylC₀₋₁₂alkyl, C₃₋₁₂cycloalkylC₀₋₁₂alkyl, heterocycloalkylC₀₋₁₂alkyl, arylC₃₋₁₂cycloalkyl, heteroarylC₃₋₁₂cycloalkyl, heterocycloalkylC₃₋₁₂cycloalkyl, C₃₋₁₂cycloalkylC₃₋₁₂cycloalkyl, C₁₋₁₂alkylheterocycloalkyl, heterocycloalkyl-heterocycloalkyl, aryl-heterocycloalkyl or heteroaryl-heterocycloalkyl, any of which is optionally substituted with one or more independent halo, —CN, —OH, —NH₂, or C₁₋₁₀alkyl which may be partially or fully halogenated, or —O—C₁₋₁₀alkyl which alkyl may be partially or fully halogenated;
each R⁴, R⁵, R⁶, R⁷, R⁸, R⁹, R¹⁰, R¹¹, R¹², R¹³, R¹⁴, R¹⁵, R^{a}, R^{b}, and R^{c} is independently selected from H, C₁₋₁₂alkyl or C₃₋₁₂cycloalkyl, each optionally substituted by halo, —OCF₃, or by —OC₀₋₃alkyl, arylC₀₋₁₂alkyl, heteroarylC₀₋₁₂alkyl, C₃₋₁₂cycloalkylC₀₋₁₂alkyl, heterocycloalkylC₀₋₁₂alkyl, arylC₃₋₁₂cycloalkyl, heteroarylC₃₋₁₂cycloalkyl, heterocycloalkylC₃₋₁₂cycloalkyl, C₃₋₁₂cycloalkylC₃₋₁₂cycloalkyl, C₁₋₁₂alkyl-heterocycloalkyl, heterocycloalkyl-heterocycloalkyl, aryl-heterocycloalkyl, or heteroaryl-heterocycloalkyl;
—NR⁴R⁵, —NR⁶R⁷ and —NR¹¹R¹² is each independently linear structure; or R⁴ and R⁵, or R⁶ and R⁷, or R¹¹ and R¹², respectively, can be taken together with the nitrogen atom to which they are attached to form a 3-12 membered saturated or unsaturated ring, wherein said ring optionally includes one or more heteroatoms selected from O, N, or S(O)ₘ;
—CR⁸R⁹ or —CR¹³R¹⁴ is each independently linear structure; or R⁸ and R⁹, or R¹³ and R¹⁴, respectively, can be taken together with the carbon atom to which they are attached to form a 3-12 membered saturated or unsaturated ring, wherein said ring optionally includes one or more heteroatoms selected from O, N, or S(O)ₘ;
n=0-7; and
m=0-2.

3. The compound or salt of claim 1 or 2, wherein:
Y₁, Y₂, Y₃, and Y₄ are CH; and Y₅ is N; or
Y₁ and Y₂ are CH; Y₃ is NH; Y₄ is N; and Y₅ is C.

4. The compound or salt of claim 1 or 2, wherein:
Y₁ is N; Y₂ and Y₄ are CH; Y₃ is NH; and Y₅ is C.

5. The compound or salt of any one of claims 1-4, wherein X is selected from —OH, C₁₋₃alkyl, or C₁₋₃alkoxy.

6. The compound or salt of any one of claim 1, 3, or 4, wherein:
R^{1a} and R^{1e} are each independently selected from halo, —CN, C₁₋₆alkyl, —CF₃, —OCF₃, —OCHF₂, or —OC₀₋₆alkyl;
R^{1b}, R^{1c}, and R^{1d} are each independently selected from H, halo, —CN, C₁₋₆alkyl, —CF₃, —OCF₃, —OCHF₂, or —OC₀₋₆alkyl; wherein the alkyl is optionally substituted with —OH, —OC₁₋₆alkyl, N(C₀₋₆alkyl)(C₀₋₆alkyl), C(═O)N(C₀₋₆alkyl)(C₀₋₆alkyl), C(═O)OC₀₋₆alkyl, C(═O)C₀₋₆alkyl, or heteroaryl;
R² is selected from halo, —CN, —CF₃, —NO₂, C₁₋₆alkyl, C₂₋₆alkenyl, C₂₋₆alkynyl, C₃₋₆cycloalkylC₀₋₆alkyl, C₃₋₆heterocycloalkylC₀₋₆alkyl, arylC₀₋₆alkyl, or heteroarylC₀₋₆alkyl, any of which is optionally substituted with 1-3 independent G¹ substituents;
or R² is selected from: 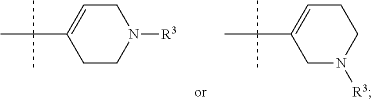
R³ is selected from H, C₁₋₁₂alkyl, R⁴O—C₂₋₁₂alkyl-, R⁴R⁵N—C₂₋₁₂alkyl-, R⁴S(O)ₘ—C₂₋₁₂alkyl-, C₃₋₁₂cycloalkylC₀₋₁₂alkyl, C₃₋₁₂cycloalkenylC₁₋₁₂alkyl, C₃₋₁₂heterocycloalkylC₀₋₁₂alkyl, arylC₀₋₁₂alkyl, heteroarylC₀₋₁₂alkyl, C₁₋₁₂alkylC₃₋₁₂cycloalkyl, C₃₋₁₂cycloalkylC₃₋₁₂cycloalkyl, C₃₋₁₂cycloalkenylC₃₋₁₂cycloalkyl, C₃₋₁₂heterocycloalkylC₃₋₁₂cycloalkyl, arylC₃₋₁₂cycloalkyl, heteroarylC₃₋₁₂cycloalkyl, C₁₋₁₂alkylC₃₋₁₂heterocycloalkyl, C₃₋₁₂cycloalkylC₃₋₁₂heterocycloalkyl, C₃₋₁₂cycloalkenylC₃₋₁₂heterocycloalkyl, C₃₋₁₂heterocycloalkylC₃₋₁₂heterocycloalkyl, arylC₃₋₁₂heterocycloalkyl, heteroarylC₃₋₁₂heterocycloalkyl, —C(O)R^{a}, R⁴O—C₀₋₁₂alkylC(O)—, R⁴R⁵N—C₀₋₁₂alkylC(O)—, R⁴S(O)ₘC₀₋₁₂alkylC(O)—, —CO₂R⁴, —C(O)NR⁴R⁵, —S(O)ₘR⁴, —SO₂NR⁴R⁵ or —C(S)OR⁴, any of which is optionally substituted with 1-2 independent G² substituents;
each G¹ is independently selected from halo, —CN, —CF₃, —OCF₃, —NO₂, R⁶, oxo, C₁₋₁₂alkyl, C₂₋₁₂alkenyl, C₂₋₁₂alkynyl, C₃₋₁₂cycloalkylC₀₋₁₂alkyl, C₃₋₁₂heterocycloalkylC₀₋₁₂alkyl, arylC₀₋₁₂alkyl, heteroarylC₀₋₁₂alkyl, —OR⁶, —S(O)ₘR⁶, —NR⁶R⁷, —SO₂NR⁶R⁷, —C(O)R^{b}, —C(O)NR⁶R⁷, —C(O)—C(O)NR⁶R⁷, —C(O)OR⁶, —C(O)—C(O)OR⁶, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, —NR¹⁰S(O)₂NR⁶R⁷, or —NR¹⁰S(O)NR⁶R⁷, any of which is optionally substituted with 1-2 independent Q¹ substituents;
each G² is independently selected from halo, —CN, —CF₃, —OCF₃, —NO₂, C₁₋₁₂alkyl, C₂₋₁₂alkenyl, C₂₋₁₂alkynyl, —OR⁶, —S(O)ₘR⁶, —NR⁶R⁷, —SO₂NR⁶R⁷, —C(O)R^{b}, —C(O)NR⁶R⁷, —C(O)—C(O)NR⁶R⁷, —C(O)OR⁶, —C(O)—C(O)OR⁶, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)NR⁶R⁷, —(CR⁸R⁹)OR⁶, —(CR⁸R⁹)S(O)ₘR⁶, —NR¹⁰C(O)NR⁶R⁷, —NR¹⁰S(O)₂NR⁶R⁷, or —NR¹⁰S(O)NR⁶R⁷, any of which is optionally substituted with 1-2 independent Q¹ substituents;
each Q¹ is selected from halo, —CN, —NO₂, oxo, —CF₃, —OCF₃, C₁₋₁₂alkyl, C₃₋₇cycloalkyl, —C(O)—C(O)NR¹¹R¹², —C(O)—C(O)OR¹¹, —OC(O)Rc, —NR¹¹C(O)Rc, —NR¹¹S(O)₂R¹², —(CR¹³R¹⁴)ₙC(O)R^{c}, —(CR¹³R¹⁴)ₙC(O)OR¹¹, —(CR¹³R¹⁴)ₙC(O)NR¹¹R¹², —(CR¹³R¹⁴)ₙS(O)₂NR¹¹R¹², —(CR¹³R¹⁴)ₙNR¹¹R¹², —(CR¹³R¹⁴)ₙOR¹¹, —(CR¹³R¹⁴)ₙS(O)ₘR¹¹, —NR¹⁵C(O)NR¹¹R¹², —NR¹⁵S(O)₂NR¹¹R¹² or —NR¹⁵S(O)NR¹¹R¹²;
each R⁴, R⁵, R⁶, R⁷, R⁸, R⁹, R¹⁰, R¹¹, R¹², R¹³, R¹⁴, R¹⁵, R^{a}, R^{b}, and R^{c} is independently C₀₋₁₂alkyl or C₃₋₇cycloalkyl, each independently optionally substituted by halo, —OCF₃, or —OC₀₋₃alkyl;
each —NR⁴R⁵, —NR⁶R⁷ and —NR¹¹R¹² is independently linear in structure; or R⁴ and R⁵, or R⁶ and R⁷, or R¹¹ and R¹², respectively, can be taken together with the nitrogen atom to which they are attached to form a 3-12 membered saturated or unsaturated ring, wherein said ring optionally includes one or more heteroatoms selected from O, N, or S(O)ₘ;
each —CR⁸R⁹ and —CR¹³R¹⁴ is independently linear in structure; or R⁸ and R⁹, or R¹³ and R¹⁴, respectively, can be taken together with the carbon atom to which they are attached to form a 3-12 membered saturated or unsaturated ring, wherein said ring optionally includes one or more heteroatoms selected from O, N, or S(O)ₘ;
n=0-4; and
m=0-2.

7. The compound or salt of any one of claim 1, 3, or 4, having the formula: 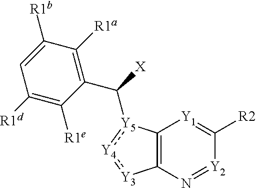
wherein X is methyl, ethyl, or methoxy;
R^{1a} and R^{1e} are each independently selected from halo, —CN, C₁₋₆alkyl, —CF₃, —OCF₃, —OCHF₂, or —OC₁₋₆alkyl;
R^{1b} and R^{1d} are each independently selected from H, halo, —CN, C₁₋₆alkyl, —CF₃, —OCF₃, —OCHF₂, or —OC₁₋₆alkyl;
(i) R² is phenyl or pyridinyl, each substituted by one or more R¹⁸ or G¹ wherein G¹ is ₄₋₇heterocycloalkyl optionally substituted with halogen, —OH, —OCH₃, or C₁₋₃alkyl, or G¹ is —C(O)NR⁶R⁷; wherein each R⁶ and R⁷ is independently C₀₋₃ alkyl; or NR⁶R⁷ defines a ₄₋₇heterocycloalkyl optionally substituted by C₁₋₆alkyl;
or (ii) R² is pyrazolo optionally substituted by one or more R¹⁸ or G¹ wherein G¹ is ₄₋₆heterocycloalkyl optionally substituted by halo, —R⁶, oxo, —S(O)ₘR⁶, —SO₂NR⁶R⁷, —C(O)R^{b}, —C(O)NR⁶R⁷, —C(O)—C(O)NR⁶R⁷, —C(O)OR⁶, or —C(O)—C(O)OR⁶; or G¹ is C₃₋₆cycloalkyl optionally substituted by halo, OH, —OR⁶, oxo, —S(O)ₘR⁶, —SO₂NR⁶R⁷, —C(O)R^{b}, —C(O)NR⁶R⁷, —C(O)—C(O)NR⁶R⁷, —C(O)OR⁶, or —C(O)—C(O)OR⁶; or —C₁₋₆alkyl which alkyl can be substituted by halo or —OC₀₋₅alkyl; or G¹ is C₁₋₆alkyl optionally substituted by —OH, —OR⁶, —R⁶, oxo, —NR⁶R⁷, —C(O)R^{b}, —C(O)NR⁶R⁷, —C(O)—C(O)NR⁶R⁷, —C(O)OR⁶, —C(O)—C(O)OR⁶, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, —NR¹⁰S(O)₂NR⁶R⁷, or —NR¹⁰S(O)NR⁶R⁷; wherein each R⁶, R⁷, R⁸, R⁹, R¹⁰, and R^{b} is independently C₀₋₅alkyl or C₃₋₆cycloalkyl, each independently optionally substituted by halo, —OCF₃, or —OC₀₋₃alkyl; or NR⁶R⁷ defines a ₄₋₇heterocycloalkyl optionally substituted by C₁₋₆alkyl; R¹⁸ is —R⁶, halo, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, or —NR¹⁰S(O)₂NR⁶R⁷; and wherein each m is independently 0-2; each n is independently 0-2.

8. The compound or salt of claim 7, wherein:
X is methyl;
R2 is pyrazole substituted by one or more R¹⁸ or G1;
R^{1a} and R^{1e} are each independently selected from halo, —CN, —CF₃, —OCF₃, —OCHF₂, or —OC₁₋₆alkyl;
R^{1b} and R^{1d} are each independently selected from H, halo, —CN, —CF₃, —OCF₃, —OCHF₂, or —OC₁₋₆alkyl;
G¹ is ₄₋₆heterocycloalkyl optionally substituted by halo, —R⁶, oxo, —S(O)ₘR⁶, —SO₂NR⁶R⁷, —C(O)R^{b}, —C(O)NR⁶R⁷, —C(O)—C(O)NR⁶R⁷, —C(O)OR⁶, or —C(O)—C(O)OR⁶;
or G¹ is ₃₋₆cycloalkyl optionally substituted by OH, —OR⁶, oxo, halo, —S(O)ₘR⁶, —SO₂NR⁶R⁷, —C(O)R^{b}, —C(O)NR⁶R⁷, —C(O)—C(O)NR⁶R⁷, —C(O)OR⁶, or —C(O)—C(O)OR⁶, or —C₁₋₆alkyl which alkyl can be substituted by halo or —OC₀₋₅alkyl;
or G¹ is C₁₋₆alkyl optionally substituted by —OH, —OR⁶, —R⁶, oxo, halo, —NR⁶R⁷, —C(O)R^{b}, —C(O)NR⁶R⁷, —C(O)—C(O)NR⁶R⁷, —C(O)OR⁶, —C(O)—C(O)OR⁶, —OC(O)R^{b}, —NR⁶C(O)R^{b}, —NR⁶S(O)₂R⁷, —(CR⁸R⁹)C(O)R^{b}, —(CR⁸R⁹)C(O)OR⁶, —(CR⁸R⁹)C(O)NR⁶R⁷, —(CR⁸R⁹)S(O)₂NR⁶R⁷, —(CR⁸R⁹)NR⁶R⁷, —(CR⁸R⁹)OR⁶, —(CR⁸R⁹)S(O)ₘR⁶, —NR¹⁰C(O)NR⁶R⁷, —NR¹⁰S(O)₂NR⁶R⁷, or —NR¹⁹S(O)NR⁶R⁷;
wherein each R⁶, R⁷, R⁸, R⁹, R¹⁰, and R^{b} is independently C₀₋₅ alkyl or C₃₋₆cycloalkyl, each independently optionally substituted by halo, —OCF₃, or —OC₀₋₃alkyl; or NR⁶R⁷ defines a ₄₋₇heterocycloalkyl optionally substituted by C₁₋₆alkyl; R¹⁸ is —R⁶, halo, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, or —NR¹⁰S(O)₂NR⁶R⁷; and
each m is independently 0-2; and each n is independently 0-2.

9. The compound or salt of claim 7, wherein:
X is methyl;
R² is pyrazole substituted by one or more R¹⁸ or G1;
R^{1a} is Cl;
R^{1e} is Cl, —OCH₃, or —OCHF₂;
each R^{1b} and R^{1d} is independently H, F, or —OCH₃;
G¹ is ₄₋₆heterocycloalkyl optionally substituted by halo, R⁶, oxo, —S(O)ₘR⁶, —SO₂NR⁶R⁷, —C(O)R^{b}, —C(O)NR⁶R⁷, —C(O)—C(O)NR⁶R⁷, —C(O)OR⁶, or —C(O)—C(O)OR⁶;
wherein each R⁶, R⁷, and R^{b} is independently C₀₋₅alkyl or C₃₋₆cycloalkyl, each independently optionally substituted by halo, —OCF₃, or —OC₀₋₃alkyl; or NR⁶R⁷ defines a ₄₋₇heterocycloalkyl optionally substituted by C₁₋₆alkyl; R¹⁸ is —R⁶, halo, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, or —NR¹⁹S(O)₂NR⁶R⁷; and
m is 0-2.

10. The compound or salt of claim 7, wherein:
X is methyl;
R2 is pyrazole substituted by one or more R¹⁸ or G1;
R^{1a} is Cl;
R^{1e} is Cl, —OCH₃, or —OCHF₂;
each R^{1b} and R^{1d} is independently H, F, or —OCH₃;
G¹ is ₃₋₆cycloalkyl substituted by 0-2 substituents independently selected from —OH, —OR⁶, oxo, halo, —S(O)ₘR⁶, —SO₂NR⁶R⁷, —C(O)R^{b}, —C(O)NR⁶R⁷, —C(O)—C(O)NR⁶R⁷, —C(O)OR⁶, —C(O)—C(O)OR⁶, or —C₁₋₃alkyl which alkyl can be substituted by halo or —OC₀₋₅alkyl;
wherein each R⁶, R⁷, and R^{b} is independently C₀₋₅ alkyl or C₃₋₆cycloalkyl; or NR⁶R⁷ defines a ₄₋₇heterocycloalkyl optionally substituted by C₁₋₆alkyl; R¹⁸ is —R⁶, halo, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, or —NR¹⁰S(O)₂NR⁶R⁷; and
m is 0-2.

11. The compound or salt of claim 7, wherein:
X is methyl;
R2 is pyrazole substituted by one or more R¹⁸ or G1;
R^{1a} is Cl;
R^{1e} is Cl, —OCH₃, or —OCHF₂;
each R^{1b} and R^{1d} is independently H, F, or —OCH₃;
G¹ is C₁₋₆alkyl substituted by 0-2 substituents independently selected from —OH, —OR⁶, —R⁶, oxo, halo, —NR⁶R⁷, —C(O)R^{b}, —C(O)NR⁶R⁷, —C(O)—C(O)NR⁶R⁷, —C(O)OR⁶, —C(O)—C(O)OR⁶, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, —NR¹⁰S(O)₂NR⁶R⁷, —NR¹⁰S(O)NR⁶R⁷, or ₄₋₇heterocycloalkyl optionally substituted by C₁₋₆alkyl;
wherein each R⁶, R⁷, R⁸, R⁹, R¹⁰, and R^{b} is independently C₀₋₅ alkyl or C₃₋₆cycloalkyl; or NR⁶R⁷ defines a ₄₋₇heterocycloalkyl optionally substituted by C₁₋₆alkyl; R¹⁸ is —R⁶, halo, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁹C(O)NR⁶R⁷, or —NR¹⁰S(O)₂NR⁶R⁷;
m is 0-2; and each n is independently 0-2.

12. The compound or salt of claim 7, wherein:
X is methyl;
R2 is pyrazole substituted by one or more R¹⁸ or G1;
R^{1a} is Cl;
R^{1e} is Cl, —OCH₃, or —OCHF₂;
R^{1b} is F or —OCH₃;
R^{1d} is H;
G¹ is C₁₋₆alkyl substituted by 0-2 substituents independently selected from —OH, —OR⁶, —R⁶, oxo, halo, —NR⁶R⁷, —C(O)R^{b}, —C(O)NR⁶R⁷, —C(O)—C(O)NR⁶R⁷, —C(O)OR⁶, —C(O)—C(O)OR⁶, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, —NR¹⁰S(O)₂NR⁶R⁷, —NR¹⁰S(O)NR⁶R⁷, or ₄₋₇heterocycloalkyl optionally substituted by C₁₋₆alkyl;
wherein each R⁶, R⁷, R⁸, R⁹, R¹⁰, and R^{b} is independently C₀₋₅ alkyl or C₃₋₆cycloalkyl; or NR⁶R⁷ defines a ₄₋₇heterocycloalkyl optionally substituted by C₁₋₆alkyl; R¹⁸ is —R⁶, halo, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, or —NR¹⁰S(O)₂NR⁶R⁷;
m is 0-2; and each n is independently 0-2.

13. The compound or salt of claim 7, wherein:
X is methyl;
R2 is pyrazole substituted by one or more R¹⁸ or G1;
R^{1a} is Cl;
R^{1e} is Cl, —OCH₃, or —OCHF₂;
R^{1b} is F;
R^{1d} is H;
G¹ is C₁₋₆alkyl substituted by 0-2 substituents independently selected from —OH, —OR⁶, —R⁶, oxo, halo; —NR⁶R⁷, —C(O)R^{b}, —C(O)NR⁶R⁷, —C(O)—C(O)NR⁶R⁷, —C(O)OR⁶, —C(O)—C(O)OR⁶, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, —NR¹⁰S(O)₂NR⁶R⁷, —NR¹⁰S(O)NR⁶R⁷, or ₄₋₇heterocycloalkyl optionally substituted by C₁₋₆alkyl;
wherein each R⁶, R⁷, R⁸, R⁹, R¹⁰, and R^{b} is independently C₀₋₃ alkyl or C₃₋₆cycloalkyl; or NR⁶R⁷ defines a ₄₋₇heterocycloalkyl optionally substituted by C₁₋₆alkyl; R¹⁸ is —R⁶, halo, —OC(O)R^{b}, —NR⁶C(O)R^{b}, —NR⁶S(O)₂R⁷, —(CR⁸R⁹)ₙC(O)R^{b}, —(CR⁸R⁹)ₙC(O)OR⁶, —(CR⁸R⁹)ₙC(O)NR⁶R⁷, —(CR⁸R⁹)ₙS(O)₂NR⁶R⁷, —(CR⁸R⁹)ₙNR⁶R⁷, —(CR⁸R⁹)ₙOR⁶, —(CR⁸R⁹)ₙS(O)ₘR⁶, —NR¹⁰C(O)NR⁶R⁷, or —NR¹⁰S(O)₂NR⁶R⁷;
m is 0-2; and each n is independently 0-2.

14. The compound or salt of claim 7, wherein:
X is methyl;
R^{1a} and R^{1e} are each independently selected from halo, —CN, C₁₋₆alkyl, —CF₃, —OCF₃, —OCHF₂, or —OC₁₋₆alkyl;
R^{1b} and R^{1d} are each independently selected from H, halo, —CN, C₁₋₆alkyl, —CF₃, —OCF₃, —OCHF₂, or —OC₁₋₆alkyl;
R² is phenyl or pyridinyl, each substituted by G¹;
G¹ is ₄₋₇heterocycloalkyl optionally substituted with halogen, —OH, —OCH₃, or C₁₋₃alkyl;
or G¹ is —C(O)NR⁶R⁷; and
each R⁶ and R⁷ is independently C₀₋₃ alkyl or C₃₋₆cycloalkyl; or NR⁶R⁷ defines a ₄₋₇heterocycloalkyl optionally substituted by C₁₋₆alkyl.

15. The compound or salt of any one of claims 1-14, which is present as a material that is substantially free of its (S)-1-(phenyl)ethyl enantiomer when Y₄ or Y₅ of Formula I is N and substantially free of its (R)-1-(phenyl)ethyl enantiomer when Y₄ or Y₅ is not N.

16. The compound or salt of any one of claims 1-15, which exhibits inhibition of MET in a cellular assay with an IC₆₀ of about 100 nM or less.

17. The compound or salt of any one of claims 1-16, which exhibits inhibition of Ron in a cellular assay with an IC₅₀ of about 500 nM or less.

18. The compound or salt of any one of claims 1-17, which is about 10-fold or more selective for MET over KDR.

19. The compound or salt of any one of claims 1-18, which is about 10-fold or more selective for MET over Aurora kinase B.

20. The compound or salt of claim 1, selected from any one of Examples 1-16 herein.

21. A pharmaceutical composition comprising the compound or salt of any one of claims 1-20, formulated with or without one or more pharmaceutical carriers.

22. Use of a therapeutically effective amount of a compound or salt of any one of claims 1-20 in the manufacture of a medicament for treating a cancer mediated at least in part by MET and/or RON or which inhibition of RON and/or MET is effective.

23. Use of a therapeutically effective amount of a compound or salt of any one of claims 1-20 in the manufacture of a medicament for treating a cancer selected from bladder, colorectal, non-small cell lung, breast, or pancreatic, ovarian, gastric, head and neck, prostate, hepatocellular, renal, glioma, or sarcoma.

24. The use of claim 22 or 23, wherein the compound or salt is a dual RON and MET inhibitor.
